(19) United States
(12) Patent Application Publication
(10) Pub. No.: US 2025/0206714 A1
(43) Pub. Date: Jun. 26, 2025

(54) COMPOSITIONS AND METHODS FOR TREATMENT OF INFLAMMATORY DISEASES

(71) Applicant: Invea Therapeutics, Inc., Guilford, CT (US)

(72) Inventors: Krishnan Nandabalan, Guilford, CT (US); Sameer Sharma, Palampur (IN); Matthew Sternke, Astoria, NY (US); Praful Gupta, Meerut (IN); Govindan Vijayadamodar, Branford, CT (US); Madan Anant, Cheshire, CT (US); Raghava Reddy Kethiri, Hyderabad (IN); Krishna Reddy Gankidi, Hyderabad (IN)

(21) Appl. No.: 19/076,752

(22) Filed: Mar. 11, 2025

Related U.S. Application Data
(63) Continuation of application No. PCT/US23/32561, filed on Sep. 12, 2023.
(60) Provisional application No. 63/426,965, filed on Nov. 21, 2022.

(30) Foreign Application Priority Data
Sep. 12, 2022 (IN) 202211051974

(51) Int. Cl. C07D 401/06; C07D 243/38; C07D 281/02; C07D 285/36; C07D 403/06; C07D 405/06

(52) U.S. Cl. CPC C07D 401/06; C07D 243/38; C07D 281/02; C07D 285/36; C07D 403/06; C07D 405/06

(57) ABSTRACT

The disclosure pertains to chemical entities such as a compound or a pharmaceutically acceptable salt that inhibit inflammatory responses by, for example, inhibiting the oligomerization of Apoptosis-associated speck-like protein containing a C-terminal caspase-recruitment domain (ASC). Also provided are methods of treating various disease states using compounds that inhibit oligomerization of ASC protein.

### RELATED APPLICATIONS

This application is a continuation application of International Patent Application No. PCT/US23/32561, filed Sep. 12, 2023, which claims priority to U.S. Provisional Application No. 63/426,965 filed on Nov. 21, 2022, and Indian provisional application No. 202211051974 filed on Sep. 12, 2022, the content of each of which is herein incorporated by reference in its entirety.

### TECHNICAL FIELD

This invention relates to chemical entities (e.g., a compound or a pharmaceutically acceptable salt thereof, and/or drug combination containing the compound), their use in the treatment of diseases involving inflammation, and their synthesis.

### BACKGROUND

Inflammation is a protective immune response mounted by the innate immune system in response to harmful stimuli, such as pathogens, dead cells or irritants, and is tightly regulated by the host. Insufficient inflammation can lead to persistent infection of pathogens, while excessive inflammation can cause chronic or systemic inflammatory diseases. Inflammasomes are a complex of proteins that play a role in initiating and controlling inflammatory responses. Excessive triggering of inflammasomes leads to unwanted inflammation and inflammatory diseases. Inflammasomes have thus been linked to a variety of autoinflammatory and autoimmune diseases, including neurodegenerative diseases such as inflammatory bowel disease, Crohn's Disease, multiple sclerosis, Alzheimer's disease, and Parkinson's disease. Controlling inflammation by regulating the activity of inflammasomes and its components is of interest.

### SUMMARY

The disclosure provides compounds having formula (I):

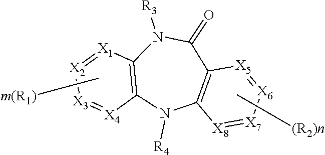

wherein R1 and R2 are each independently selected from the group consisting of hydrogen, —CO-alkyl, hydroxyl, halo, halo alkyl (C1-C6), trihalo alkyl (C1-C6), halo alkoxy, amino, C1-C6-alkyl-amino; wherein m and n are integers having each independently a value of 0, 1, 2, 3 or 4;
wherein X1, X2, X3, X4, X5, X6, X7 and X8 is each independently selected from the group consisting of —CH and N; wherein R3 is each independently selected from the group consisting of hydrogen, C1-C6 alkyl, tri-halo alkyl (C1-C6), —CO-alkyl, and —CO-haloalkyl;
wherein R4 is each independently one of hydrogen or COY, with the proviso that R4 is not hydrogen when R3 is hydrogen and when X1-X8 is —CH, except that R4 and R3 may both be hydrogen when X1-X8 is N and when R1 or R2 is a halo or

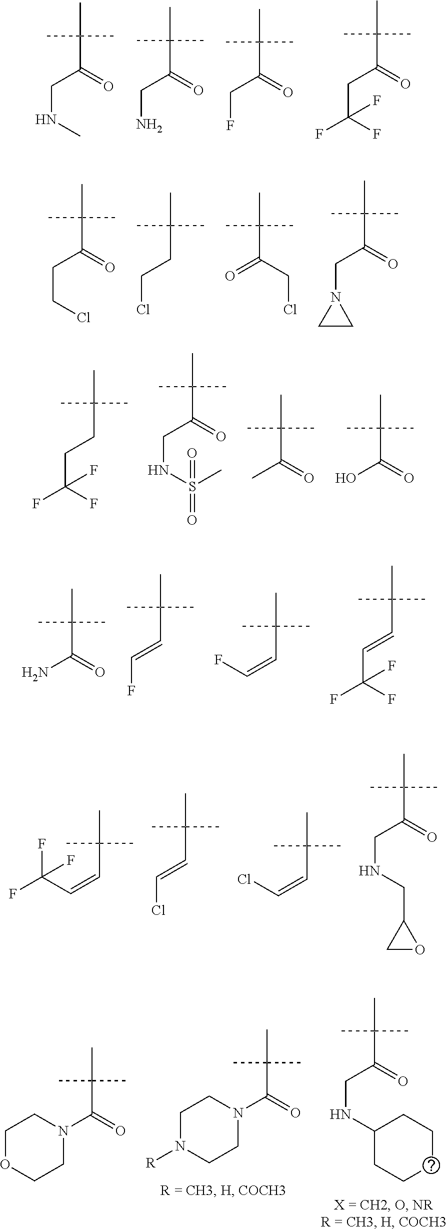

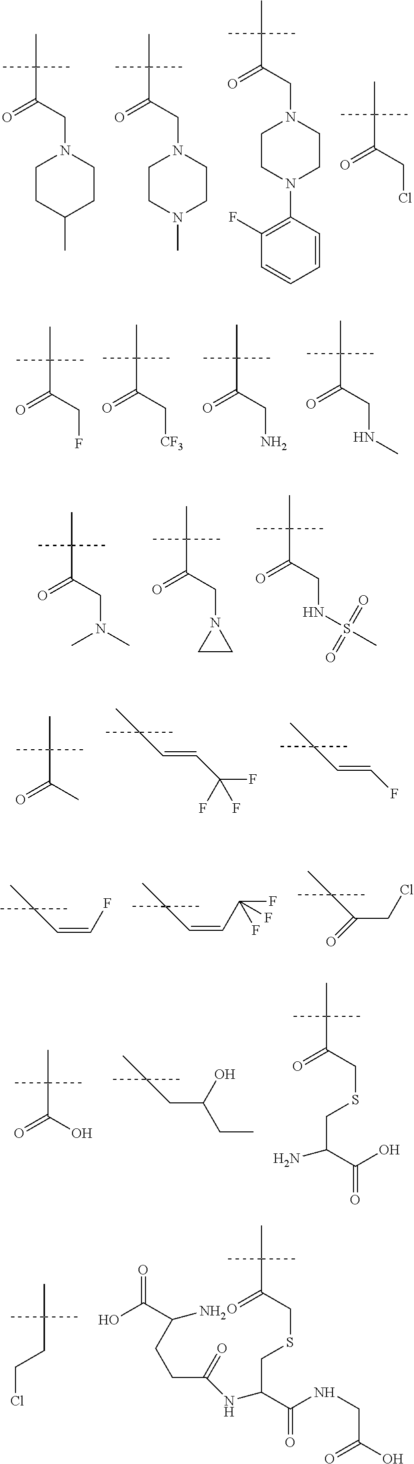

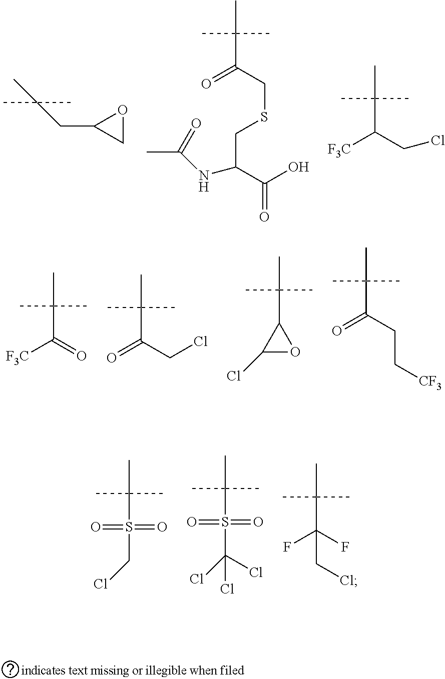

wherein Y is each independently selected from the group consisting of hydrogen, C1-C6 halo alkyl, C1-C6 halo alkoxy, C1-C6 amino alkyl, C1-C6 amino alkoxy, C3-C8 cyclo-(halo)-alkyl and wherein the alkyl or cycloalkyl group is optionally substituted with a five- or six-membered ring optionally containing at least one heteroatom selected from N, S and O, and wherein the five- or six-membered ring is optionally mono- or poly-substituted with C1-C6 alkyl, halo, C1-C6 halo alkyl, C1-C6 halo alkoxy, C1-C6 amino alkyl, C1-C6 amino alkoxy, C3-C8 cycloalkyl, C3-C8 cycloalkyl substituted with halo, amino, carboxyl or alkoxy group.

In some embodiments, the compounds of Formula I are further restricted. For example, in any of the aforesaid compounds of Formula I, R4 is not hydrogen, m is zero and n is 1. In some embodiments, R4 is COY and Y is a substituted piperazine. In some embodiments, the R4 is COY and Y is a halo alkyl. In some embodiments, R4 is hydrogen, m is zero, n is one and R2 is halo. In some embodiments, R4 is COY and Y is a substituted piperidine.

In some embodiments, the disclosure provides a compound of formula I(a)

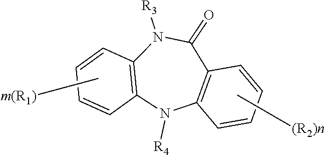

wherein R1 and R2 are each independently selected from the group consisting of hydrogen, —CO-alkyl, hydroxyl, halo, halo alkyl (C1-C6), trihalo alkyl (C1-C6), halo alkoxy, amino, C1-C6-alkyl-amino; wherein m and n are integers having each independently a value of 0, 1, 2, 3 or 4;
wherein R3 is each independently selected from the group consisting of hydrogen, C1-C6 alkyl, trilhalo alkyl (C1-C6), and —CO-alkyl;
wherein R4 is each independently one of hydrogen or COY with the proviso that R4 is not hydrogen when R3 is hydrogen, except that R4 and R3 may both be hydrogen when either R1 or R2 is halo or

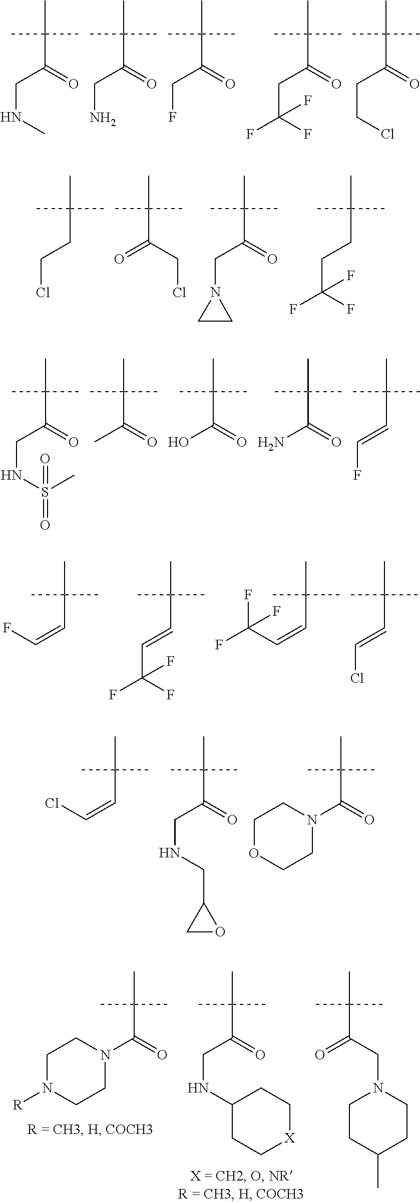

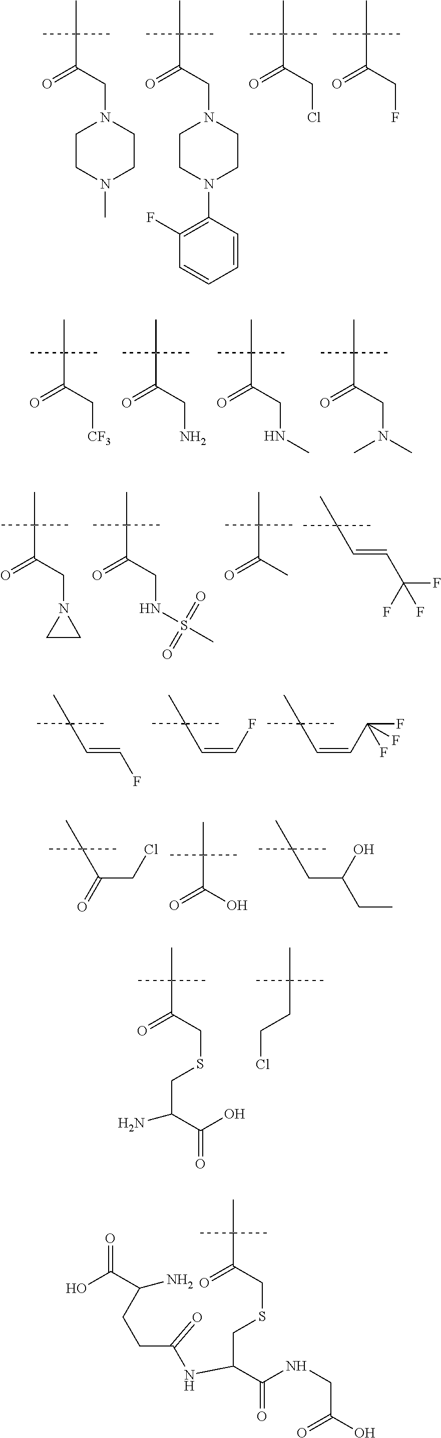

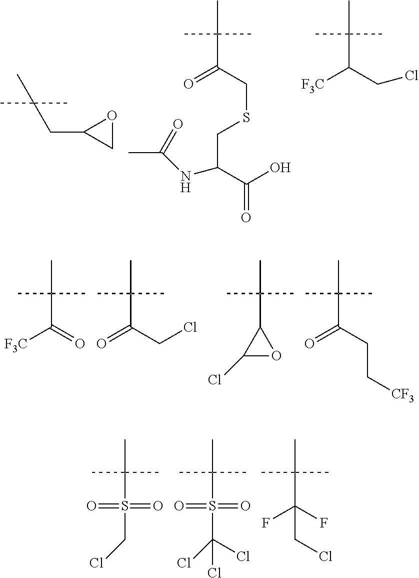

wherein Y is each independently selected from the group consisting of hydrogen, C1-C6 halo alkyl, C1-C6 halo alkoxy, C1-C6 amino alkyl, C1-C6 amino alkoxy, C3-C8 cyclo-(halo)-alkyl and wherein the alkyl or cycloalkyl group is optionally substituted with a five- or six-membered ring optionally containing at least one heteroatom selected from N, S and O, and wherein the five- or six-membered ring is optionally mono- or poly-substituted with C1-C6 alkyl, halo, C1-C6 halo alkyl, C1-C6 halo alkoxy, C1-C6 amino alkyl, C1-C6 amino alkoxy, C3-C8 cycloalkyl, C3-C8 cycloalkyl substituted with halo, amino, carboxyl or alkoxy group.
   In some embodiments the disclosure provides a compound of formula I(b)

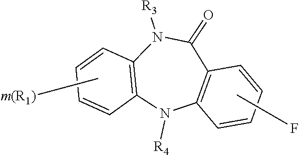

wherein R1 is each independently selected from the group consisting of hydrogen, —CO— alkyl, hydroxyl, halo, halo alkyl (C1-C6), trihalo alkyl (C1-C6), halo alkoxy, amino, C1-C6-alkyl-amino; wherein m is an integer having each independently a value of 0, 1, 2, 3 or 4;
wherein R3 is each independently selected from the group consisting of hydrogen, C1-C6 alkyl, trilhalo alkyl (C1-C6), and —CO-alkyl;
wherein R4 is each independently one of hydrogen or COY or

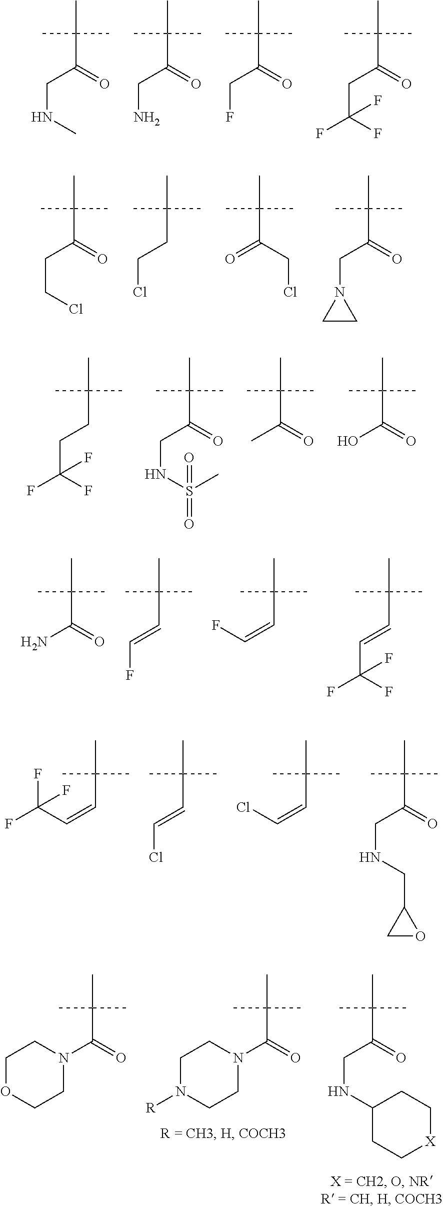

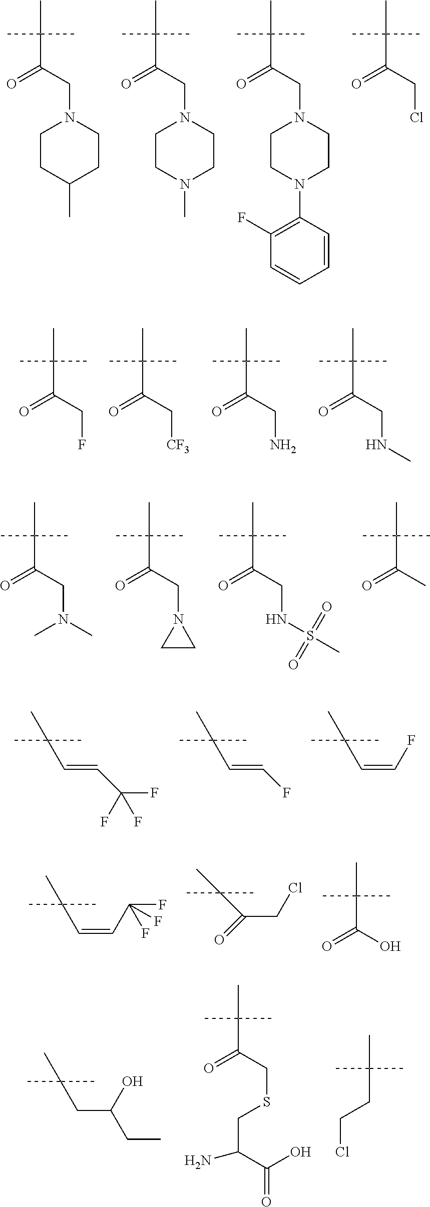

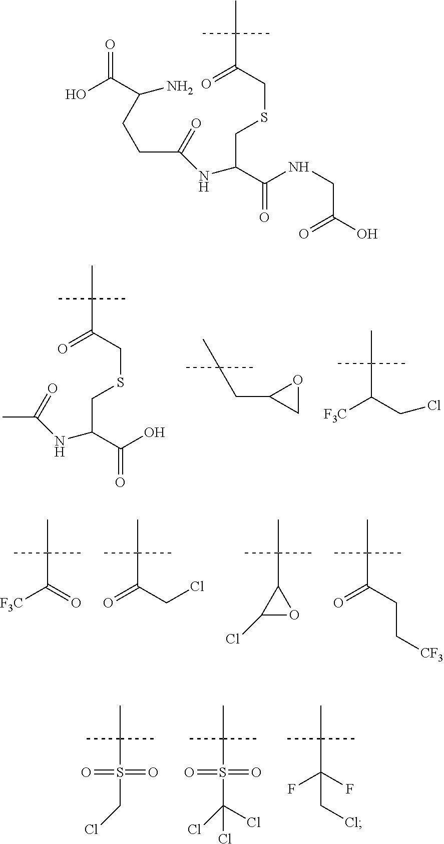

wherein Y is each independently selected from the group consisting of hydrogen, C1-C6 halo alkyl, C1-C6 halo alkoxy, C1-C6 amino alkyl, C1-C6 amino alkoxy, C3-C8 cyclo-(halo)-alkyl and wherein the alkyl or cycloalkyl group is optionally substituted with a five- or six-membered ring optionally containing at least one heteroatom selected from N, S and O, and wherein the five- or six-membered ring is optionally mono- or poly-substituted with C1-C6 alkyl, halo, C1-C6 halo alkyl, C1-C6 halo alkoxy, C1-C6 amino alkyl, C1-C6 amino alkoxy, C3-C8 cycloalkyl, C3-C8 cycloalkyl substituted with halo, amino, carboxyl or alkoxy group.
In some embodiments the disclosure provides a compound of formula I(c)

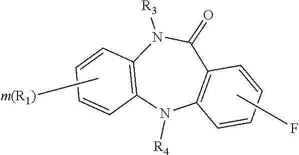

wherein R1 is hydrogen and m is 1, wherein R3 is hydrogen, and wherein R4 is each independently one of hydrogen or COY or

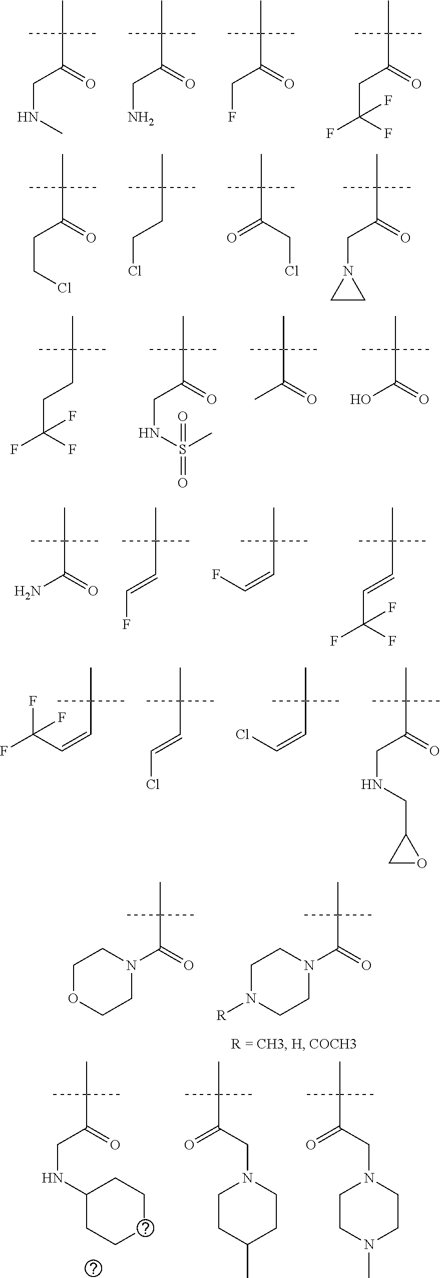

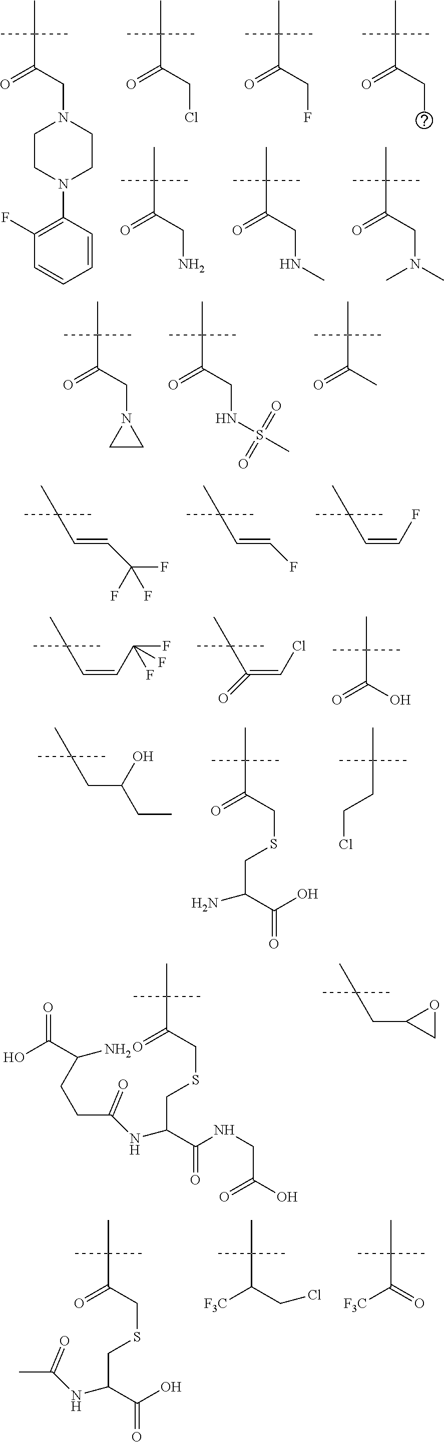

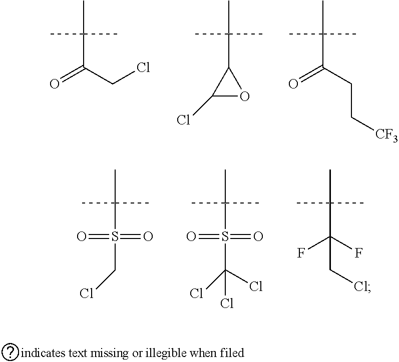

wherein Y is each independently selected from the group consisting of hydrogen, C1-C6 halo alkyl, C1-C6 halo alkoxy, C1-C6 amino alkyl, C1-C6 amino alkoxy, C3-C8 cyclo-(halo)-alkyl and wherein the alkyl or cycloalkyl group is optionally substituted with a five- or six-membered ring optionally containing at least one heteroatom selected from N, S and O, and wherein the five- or six-membered ring is optionally mono- or poly-substituted with C1-C6 alkyl, halo, C1-C6 halo alkyl, C1-C6 halo alkoxy, C1-C6 amino alkyl, C1-C6 amino alkoxy, C3-C8 cycloalkyl, C3-C8 cycloalkyl substituted with halo, amino, carboxyl or alkoxy group.

In some embodiments, the compound is selected from the group consisting of:

| Name | Compound Structure | IUPAC Name |
|---|---|---|
| Compound 1 | 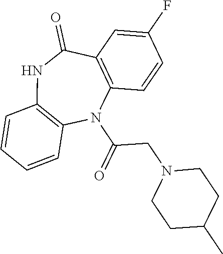 [SMILES: CC1CCN(CC(=O)N2c3ccccc3NC(=O)c3cc(F)ccc32)CC1] | 2-fluoro-5-(2-(4- methylpiperidin-1-yl)acetyl)- 5,10-dihydro-11H- dibenzo[b,e][1,4]diazepin-11- one |
| Compound 2 | 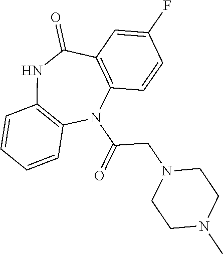 [SMILES: CN1CCN(CC(=O)N2c3ccccc3NC(=O)c3cc(F)ccc32)CC1] | 2-fluoro-5-(2-(4- methylpiperidin-1-yl)acetyl)- 5,10-dihydro-11H- dibenzo[b,e][1,4]diazepin-11- one |
| Compound 3 | 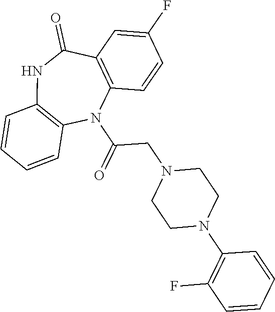 [SMILES: O=C1Nc2ccccc2N(C(=O)CN2CCN(c3ccccc3F)CC2)c2ccc(F)cc21] | 2-fluoro-5-(2-(4-(2- fluorophenyl)piperazin-1- yl)acetyl)-5,10-dihydro-11H- dibenzo[b,e][1,4]diazepin-11- one |
| Compound 4 | 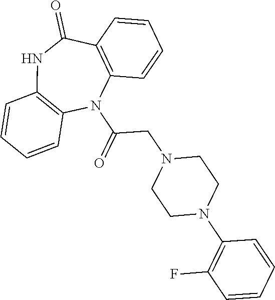 [SMILES: O=C1Nc2ccccc2N(C(=O)CN2CCN(c3ccccc3F)CC2)c2ccccc21] | 5-(2-chloroacetyl)-5,10-dihydro- 11H-dibenzo[b,e][1,4]diazepin- 11-one |
| Compound 5 | 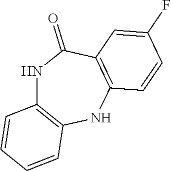 [SMILES: O=C1Nc2ccccc2Nc2ccc(F)cc21] | 2-fluoro-5,10-dihydro-11H- dibenzo[b,e][1,4]diazepin-11- one |
| Compound 6 | 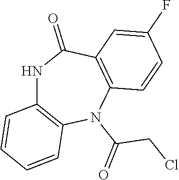 [SMILES: O=C1Nc2ccccc2N(C(=O)CCl)c2ccc(F)cc21] | 5-(2-chloroacetyl)-2-fluoro- 5,10-dihydro-11 H- dibenzo[b,e][1,4]diazepin-11- one |
| Compound 7 | 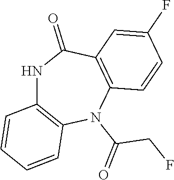 [SMILES: O=C1Nc2ccccc2N(C(=O)CF)c2ccc(F)cc21] | 2-fluoro-5-(fluoroacetyl)-5,10- dihydro-11H- dibenzo[b,e][1,4]diazepin-11- one |
| Compound 8 | 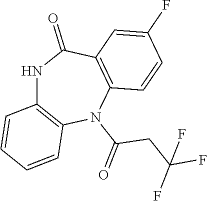 [SMILES: O=C1Nc2ccccc2N(C(=O)CC(F)(F)F)c2ccc(F)cc21] | 2-fluoro-5-(3,3,3- trifluoropropanoyl)-5,10- dihydro-11H- dibenzo[b,e][1,4]diazepin-11- one |
| Compound 9 | 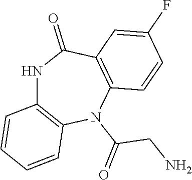 [SMILES: NCC(=O)N1c2ccccc2NC(=O)c2cc(F)ccc21] | 5-(aminoacetyl)-2-fluoro-5,10- dihydro-11H- dibenzo[b,e][1,4]diazepin-11- one |
| Compound 10 | 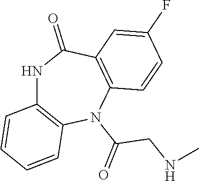 [SMILES: CNCC(=O)N1c2ccccc2NC(=O)c2cc(F)ccc21] | 2-fluoro-5- [(methylamino)acetyl]-5,10- dihydro-11H- dibenzo[b,e][1,4]diazepin-11- one |
| Compound 11 | 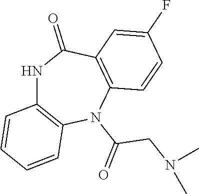 [SMILES: CN(C)CC(=O)N1c2ccccc2NC(=O)c2cc(F)ccc21] | 5-[(dimethylamino)acetyl]-2- fluoro-5,10-dihydro-11H- dibenzo[b,e][1,4]diazepin-11- one |
| Compound 12 | 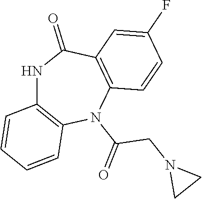 [SMILES: O=C1Nc2ccccc2N(C(=O)CN2CC2)c2ccc(F)cc21] | 5-[(aziridin-1-yl)acetyl]-2- fluoro-5,10-dihydro-11H- dibenzo[b,e][1,4]diazepin-11- one |
| Compound 13 | 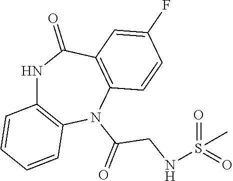 [SMILES: CS(=O)(=O)NCC(=O)N1c2ccccc2NC(=O)c2cc(F)ccc21] | N-[2-(2-fluoro-11-oxo-10,11- dihydro-5H- dibenzo[b,e][1,4]diazepin-5-yl)- 2-oxoethyl]methanesulfonamide |
| Compound 14 | 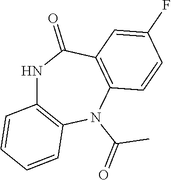 [SMILES: CC(=O)N1c2ccccc2NC(=O)c2cc(F)ccc21] | 5-acetyl-2-fluoro-5,10-dihydro- 11H-dibenzo[b,e][1,4]diazepin- 11-one |
| Compound 15 | 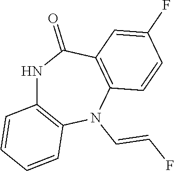 [SMILES: O=C1Nc2ccccc2N(/C=C/F)c2ccc(F)cc21] | 2-fluoro-5-[(E)-2- fluoroethenyl]-5,10-dihydro- 11H-dibenzo[b,e][1,4]diazepin- 11-one |
| Compound 16 | 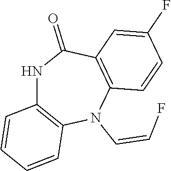 [SMILES: O=C1Nc2ccccc2N(/C=C\F)c2ccc(F)cc21] | 2-fluoro-5-[(Z)-2- fluoroethenyl]-5,10-dihydro- 11H-dibenzo[b,e][1,4]diazepin- 11-one |
| Compound 17 | 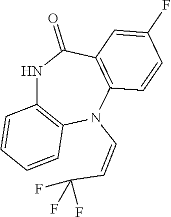 [SMILES: O=C1Nc2ccccc2N(/C=C\C(F)(F)F)c2ccc(F)cc21] | 2-fluoro-5-[(1Z)-3,3,3- trifluoroprop-1-en-1-y1]-5,10- dihydro-11H- dibenzo[b,e][1,4]diazepin-11- one |
| Compound 18 | 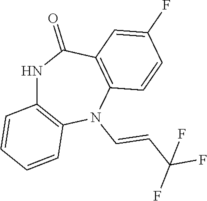 [SMILES: O=C1Nc2ccccc2N(/C=C/C(F)(F)F)c2ccc(F)cc21] | 2-fluoro-5-[(1E)-3,3,3- trifluoroprop-1-en-1-y1]-5,10- dihydro-11H- dibenzo[b,e][1,4]diazepin-11- one |
| Compound 19 | 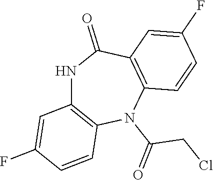 [SMILES: O=C1Nc2cc(F)ccc2N(C(=O)CCl)c2ccc(F)cc21] | 5-(chloroacetyl)-2-fluoro-10- methyl-5,10-dihydro-11H- dibenzo[b,e][1,4]diazepin-11- one |
| Compound 20 | 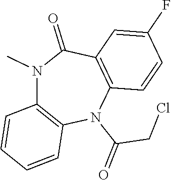 [SMILES: CN1C(=O)c2cc(F)ccc2N(C(=O)CCl)c2ccccc21] | 5-(chloroacetyl)-2-fluoro-10- methyl-5,10-dihydro-11H- dibenzo[b,e][1,4]diazepin-11- one |
| Compound 21 | 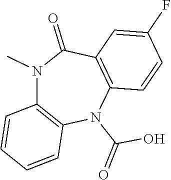 [SMILES: CN1C(=O)c2cc(F)ccc2N(C(=O)O)c2ccccc21] | 2-fluoro-10-methyl-11-oxo- 10,11-dihydro-5H- dibenzo[b,e][1,4]diazepine-5- carboxylic acid |
| Compound 22 | 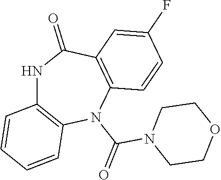 [SMILES: O=C1Nc2ccccc2N(C(=O)N2CCOCC2)c2ccc(F)cc21] | 2-fluoro-5-(morpholine-4- carbonyl)-5,10-dihydro-11H- dibenzo[b,e][1,4]diazepin-11- one |
| Compound 23 | 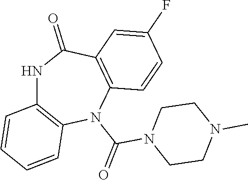 [SMILES: CN1CCN(C(=O)N2c3ccccc3NC(=O)c3cc(F)ccc32)CC1] | 2-fluoro-5-(4-methylpiperazine- 1-carbonyl)-5,10-dihydro-11H- dibenzo[b,e][1,4]diazepin-11- one |
| Compound 24 | 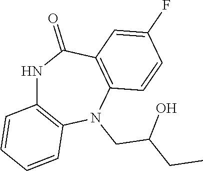 [SMILES: CCC(O)CN1c2ccccc2NC(=O)c2cc(F)ccc21] | 2-fluoro-5-(2-hydroxybutyl)- 5,10-dihydro-11H- dibenzo[b,e][1,4]diazepin-11- one |
| Compound 25 | 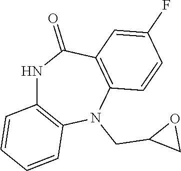 [SMILES: O=C1Nc2ccccc2N(CC2CO2)c2ccc(F)cc21] | 2-fluoro-5-[(oxiran-2- yl)methyl]-5,10-dihydro-11H- dibenzo[b,e][1,4]diazepin-11- one |
| Compound 26 | 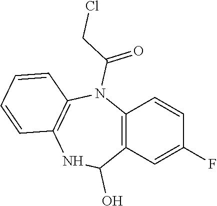 [SMILES: O=C(CCl)N1c2ccccc2NC(O)c2cc(F)ccc21] | 2-chloro-1-(2-fluoro-11- hydroxy-10,11-dihydro-5H- dibenzo[b,e][1,4]diazepin-5- yl)ethan-1-one |
| Compound 27 | 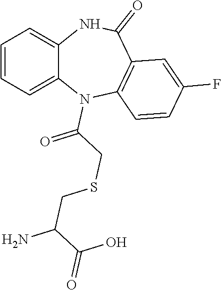 [SMILES: NC(CSCC(=O)N1c2ccccc2NC(=O)c2cc(F)ccc21)C(=O)O] | 2-amino-3-{ [2-(2-fluoro-11- oxo-10,11-dihydro-5H- dibenzo[b,e][1,4]diazepin-5-yl)- 2-oxoethyl] sulfanyl} propanoic acid |
| Compound 28 | 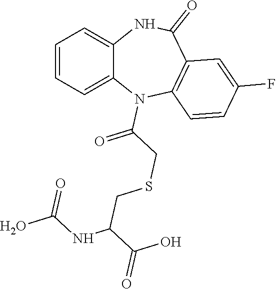 [SMILES: O=C(O)NC(CSCC(=O)N1c2ccccc2NC(=O)c2cc(F)ccc21)C(=O)O] | 2-acetamido-3-{[2-(2-fluoro-11- oxo-10,11-dihydro-5H- dibenzo[b,e][1,4]diazepin-5-y1)- 2-oxoethyl]sulfanyl } propanoic acid |
| Compound 29 | 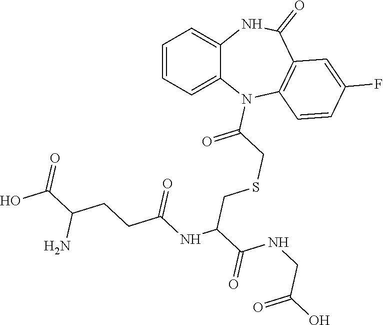 [SMILES: NC(CCC(=O)NC(CSCC(=O)N1c2ccccc2NC(=O)c2cc(F)ccc21)C(=O)NCC(=O)O)C(=O)O] | 2-amino-5-((1- ((carboxymethyl)amino)-3-((2- (2-fluoro-11-oxo-10,11-dihydro- 5H-dibenzo[b,e][1,4]diazepin-5- yl)-2-oxoethyl)thio)-1- oxopropan-2-yl)amino)-5- oxopentanoic acid |
| Compound 30 | 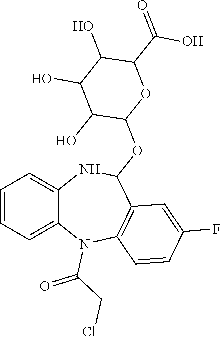 [SMILES: O=C(O)C1OC(OC2Nc3ccccc3N(C(=O)CCl)c3ccc(F)cc32)C(O)C(O)C1O] | 6-((5-(2-chloroacetyl)-2-fluoro- 10,11-dihydro-5H- dibenzo[b,e][1,4]diazepin-11- yl)oxy)-3,4,5- trihydroxytetrahydro-2H-pyran- 2-carboxylic acid |
| Compound 31 | 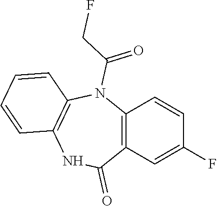 [SMILES: O=C1Nc2ccccc2N(C(=O)CF)c2ccc(F)cc21] | 2-fluoro-5-(fluoroacetyl)-5,10- dihydro-11H- dibenzo[b,e][1,4]diazepin-11- one |
| Compound 32 | 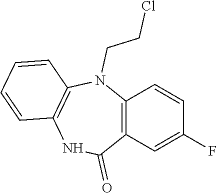 [SMILES: O=C1Nc2ccccc2N(CCCl)c2ccc(F)cc21] | 5-(2-chloroethyl)-2-fluoro-5,10- dihydro-11H- dibenzo[b,e][1,4]diazepin-11- one |
| Compound 33 | 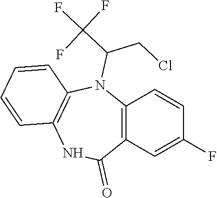 [SMILES: O=C1Nc2ccccc2N(C(CCl)C(F)(F)F)c2ccc(F)cc21] | 5-(3-chloro-1,1,1- trifluoropropan-2-yl)-2-fluoro- 5,10-dihydro-11H- dibenzo[b,e][1,4]diazepin-11- one |
| Compound 34 | 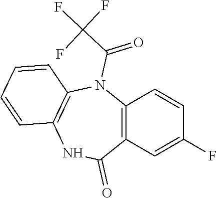 [SMILES: O=C1Nc2ccccc2N(C(=O)C(F)(F)F)c2ccc(F)cc21] | 2-fluoro-5-(trifluoroacetyl)- 5,10-dihydro-11H- dibenzo[b,e][1,4]diazepin-11- one |
| Compound 35 | 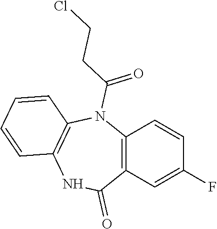 [SMILES: O=C1Nc2ccccc2N(C(=O)CCCl)c2ccc(F)cc21] | 5-(3-chloropropanoyl)-2-fluoro- 5,10-dihydro-11H- dibenzo[b,e][1,4]diazepin-11- one |
| Compound 36 | 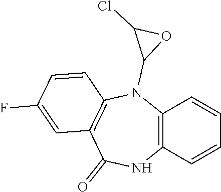 [SMILES: O=C1Nc2ccccc2N(C2OC2Cl)c2ccc(F)cc21] | 5-(3-chlorooxiran-2-y1)-2- fluoro-5,10-dihydro-11H- dibenzo[b,e][1,4]diazepin-11- one |
| Compound 37 | 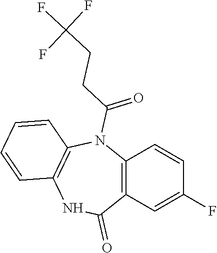 [SMILES: O=C1Nc2ccccc2N(C(=O)CCC(F)(F)F)c2ccc(F)cc21] | 2-fluoro-5-(4,4,4- trifluorobutanoyl)-5,10-dihydro- 11H-dibenzo[b,e][1,4]diazepin- 11-one |
| Compound 38 | 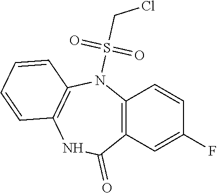 [SMILES: O=C1Nc2ccccc2N(S(=O)(=O)CCl)c2ccc(F)cc21] | 5-(chloromethanesulfonyl)-2- fluoro-5,10-dihydro-11H- dibenzo[b,e][1,4]diazepin-11- one |
| Compound 39 | 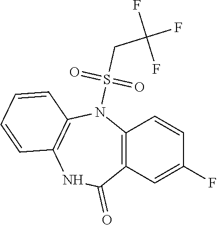 [SMILES: O=C1Nc2ccccc2N(S(=O)(=O)CC(F)(F)F)c2ccc(F)cc21] | 2-fluoro-5-(2,2,2- trifluoroethanesulfonyl)-5,10- dihydro-11H- dibenzo[b,e][1,4]diazepin-11- one |
| Compound 40 | 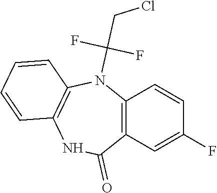 [SMILES: O=C1Nc2ccccc2N(C(F)(F)CCl)c2ccc(F)cc21] | 5-(2-chloro-1,1-difluoroethyl)-2- fluoro-5,10-dihydro-11H- dibenzo[b,e][1,4]diazepin-11- one |
| Compound 41 | 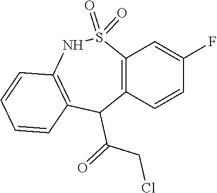 [SMILES: O=C(CCl)C1c2ccccc2NS(=O)(=O)c2cc(F)ccc21] | 11-(chloroacetyl)-3-fluoro-6,11- dihydro-5H-5λ6- dibenzo[c,f][1,2]thiazepine-5,5- dione |
| Compound 42 | 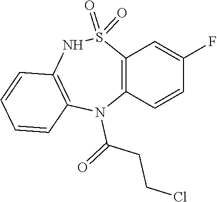 [SMILES: O=C(CCCl)N1c2ccccc2NS(=O)(=O)c2cc(F)ccc21] | 11-(3-chloropropanoyl)-3- fluoro-6,11-dihydro-5H-5λ6- dibenzo[c,f][1,2,5]thiadiazepine- 5,5-dione |
| Compound 43 | 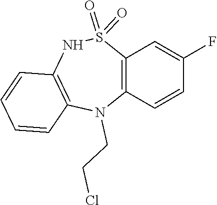 [SMILES: O=S1(=O)Nc2ccccc2N(CCCl)c2ccc(F)cc21] | 11-(2-chloroethyl)-3-fluoro- 6,11-dihydro-5H-5λ6- dibenzo[c,f][1,2,5]thiadiazepine- 5,5-dione |
| Compound 44 | 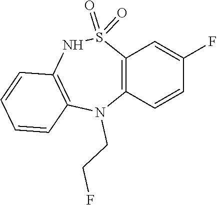 [SMILES: O=S1(=O)Nc2ccccc2N(CCF)c2ccc(F)cc21] | 3-fluoro-11-(2-fluoroethyl)- 6,11-dihydro-5H-5λ6- dibenzo[c,f][1,2,5]thiadiazepine- 5,5-dione |
| Compound 45 | 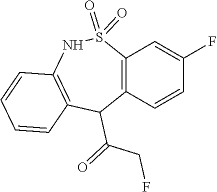 [SMILES: O=C(CF)C1c2ccccc2NS(=O)(=O)c2cc(F)ccc21] | 3-fluoro-11-(fluoroacetyl)-6,11- dihydro-5H-5λ6- dibenzo[c,f][1,2,5]thiadiazepine- 5,5-dione |
| Compound 46 | 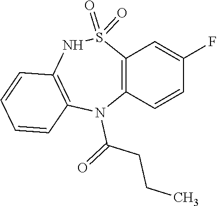 [SMILES: CCCC(=O)N1c2ccccc2NS(=O)(=O)c2cc(F)ccc21] | 11-butanoy1-3-fluoro-6,11- dihydro-5H-5λ6- dibenzo[c,f][1,2,5]thiadiazepine- 5,5-dione |
| Compound 47 | 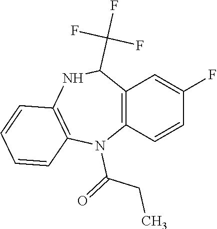 [SMILES: CCC(=O)N1c2ccccc2NC(C(F)(F)F)c2cc(F)ccc21] | 1-[2-fluoro-11- (trifluoromethyl)-10,11-dihydro- 5H-dibenzo[b,e][1,4]diazepin-5- yl]propan-1-one |
| Compound 48 | 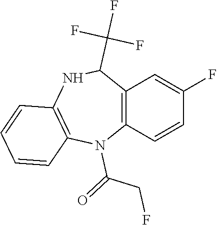 [SMILES: O=C(CF)N1c2ccccc2NC(C(F)(F)F)c2cc(F)ccc21] | 2-fluoro-1-[2-fluoro-11- (trifluoromethyl)-10,11-dihydro- 5H-dibenzo[b,e][1,4]diazepin-5- yl]ethan-1-one |
| Compound 49 | 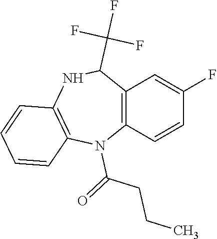 [SMILES: CCCC(=O)N1c2ccccc2NC(C(F)(F)F)c2cc(F)ccc21] | 1-[2-fluoro-11- (trifluoromethyl)-10,11-dihydro- 5H-dibenzo[b,e][1,4]diazepin-5- yl]butan-1-one |
| Compound 50 | 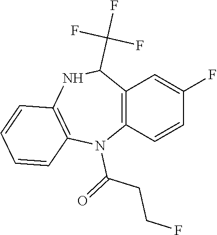 [SMILES: O=C(CCF)N1c2ccccc2NC(C(F)(F)F)c2cc(F)ccc21] | 3-fluoro-1-[2-fluoro-11- (trifluoromethyl)-10,11-dihydro- 5H-dibenzo[b,e][1,4]diazepin-5- yl]propan-1-one |
| Compound 51 | 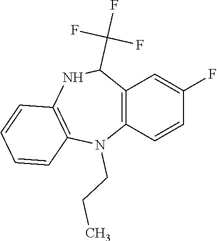 [SMILES: CCCN1c2ccccc2NC(C(F)(F)F)c2cc(F)ccc21] | 2-fluoro-5-propyl-11- (trifluoromethyl)-10,11-dihydro- 5H-dibenzo[b,e][1,4]diazepine |
| Compound 52 | 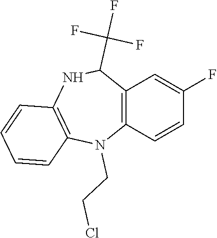 [SMILES: Fc1ccc2c(c1)C(C(F)(F)F)Nc1ccccc1N2CCCl] | 5-(2-chloroethyl)-2-fluoro-11- (trifluoromethyl)-10,11-dihydro- 5H-dibenzo[b,e][1,4]diazepine |
| Compound 53 | 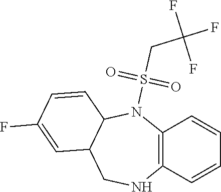 [SMILES: O=S(=O)(CC(F)(F)F)N1c2ccccc2NCC2C=C(F)C=CC21] | 2-fluoro-5-(2,2,2- trifluoroethanesulfonyl)- 5,10,11,11a-tetrahydro-4aH- dibenzo[b,e][1,4]diazepine |
| Compound 54 | 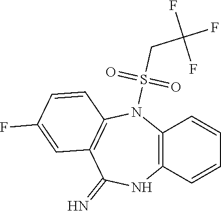 [SMILES: N=C1Nc2ccccc2N(S(=O)(=O)CC(F)(F)F)c2ccc(F)cc21] | 2-fluoro-5-(2,2,2- trifluoroethanesulfonyl)-5,10- dihydro-11H- dibenzo[b,e][1,4]diazepin-11- imine |
| Compound 55 | 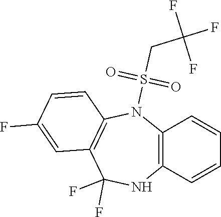 [SMILES: O=S(=O)(CC(F)(F)F)N1c2ccccc2NC(F)(F)c2cc(F)ccc21] | 2,11,11-trifluoro-5-(2,2,2- trifluoroethanesulfonyl)-10,11- dihydro-5H- dibenzo[b,e][1,4]diazepine |
| Compound 56 | 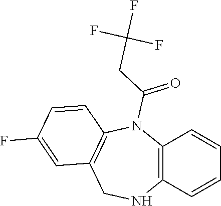 [SMILES: O=C(CC(F)(F)F)N1c2ccc(F)cc2CNc2ccccc21] | 3,3,3-trifluoro-1-(2-fluoro- 10,11-dihydro-5H- dibenzo[b,e][1,4]diazepin-5- yl)propan-1-one |
| Compound 57 | 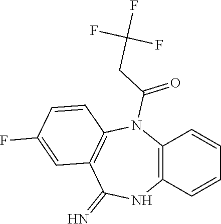 [SMILES: N=C1Nc2ccccc2N(C(=O)CC(F)(F)F)c2ccc(F)cc21] | 3,3,3-trifluoro-1-(2-fluoro-11- 5 imino-10,11-dihydro-5H- dibenzo[b,e][1,4]diazepin-5- yl)propan-1-one |
| Compound 58 | 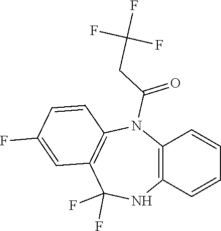 [SMILES: O=C(CC(F)(F)F)N1c2ccccc2NC(F)(F)c2cc(F)ccc21] | 3,3,3-trifluoro-1-(2,11,11- trifluoro-10,11-dihydro-5H- dibenzo[b,e][1,4]diazepin-5- yl)propan-1-one |
| Compound 59 | 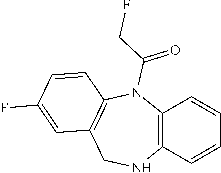 [SMILES: O=C(CF)N1c2ccc(F)cc2CNc2ccccc21] | 2-fluoro-1-(2-fluoro-10,11- dihydro-5H- dibenzo[b,e][1,4]diazepin-5- yl)ethan-1-one |
| Compound 60 | 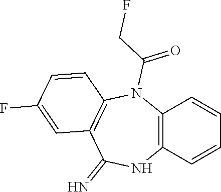 [SMILES: N=C1Nc2ccccc2N(C(=O)CF)c2ccc(F)cc21] | 2-fluoro-1-(2-fluoro-11-imino- 10,11-dihydro-5H- dibenzo[b,e][1,4]diazepin-5- yl)ethan-1-one |
| Compound 61 | 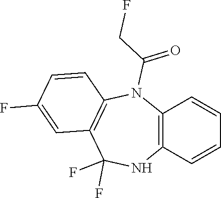 [SMILES: O=C(CF)N1c2ccccc2NC(F)(F)c2cc(F)ccc21] | 2-fluoro-1-(2,11,11-trifluoro- 10,11-dihydro-5H- dibenzo[b,e][1,4]diazepin-5- yl)ethan-1-one |
| Compound 62 | 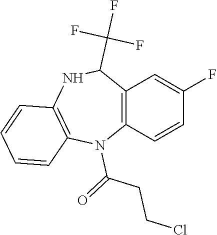 [SMILES: O=C(CCCl)N1c2ccccc2NC(C(F)(F)F)c2cc(F)ccc21] | 3-chloro-1-[2-fluoro-11- (trifluoromethyl)-10,11-dihydro- 5H-dibenzo[b,e][1,4]diazepin-5- yl]propan-1-one |

In some embodiments, one or more of the aforesaid compounds has a half maximal inhibitory concentration (IC50) value of about 2 μM. In some embodiments, the compound is capable of reducing the expression of IL-1β by at least 50%. In some embodiments, the compound can treat inflammatory diseases.

In one aspect, the disclosure provides a method of treating diseases caused by inflammation comprising administering any of the aforesaid compounds and thereby treating said disease. In some embodiments, the disease may be any one of inflammatory bowel disease (IBD), irritable bowel syndrome (IBS), Primary Sclerosing Cholangitis, primary biliary cirrhosis, alcoholic hepatitis, alcoholic liver cirrhosis, pancreatitis, non-alcoholic steatohepatitis, alcoholic pancreatitis, acute hepatitis, celiac disease, Non-steroidal anti-inflammatory drug (NSAID)-induced ulcer, gastric ulcer, antiphospholipid syndrome, Barrett's esophagus, postoperative ileus, atrophic gastritis, peritonitis, diverticulitis, duodenal ulcer, alveolar periostitis, Crohn's disease, Alzheimer's disease, arthritis, metabolic syndrome related obese, and multiple sclerosis. In other embodiments, diseases treatable with the compounds of the invention may relate to the brain or central nervous system (CNS), including Parkinson's Disease, mechanical allodynia, spinal cord injuries, Alzheimer's Disease, CNS injury, anxiety, febrile convulsions, depression, Encephalo-Myelitis, cerebro-vascular accident, subarachnoid hemorrhage, hyperactive behavior, idiopathic scoliosis, middle cerebral artery occlusion, ischemic stroke, and bipolar disorder. In still other embodiments, the diseases relate to bone, including arthritis (including rheumatoid, gouty, psoriatic), osteoarthritis, osteopenia, osteoporosis, Ankylosing Spondylitis, and intervertebral disc degeneration.

Additional embodiments involve using the compounds of the invention in the treatment of disease states relating to the eyes, the heart and vascular system, the kidneys, and the lungs, including diabetic retinopathy, dry eye syndromes, Keratoconjunctivitis Sicca, age-related macular degeneration, heart failure, myocardial infarction, myocardial reperfusion injury, coronary heart disease, myocarditis, diabetic cardiomyopathies, cardiomyopathies, cardiac fibrosis, atrial fibrillation, hypertensive disease, vasculitis, acute kidney injury, diabetic nephropathies, glomerulo-nephritis, iga glomerulo-nephritis, chronic kidney failure, lupus nephritis, nephritis, hyperuricemia, aristolochic acid nephropathy, obesity-related glomerulopathy, pulmonary fibrosis, asthma, chronic obstructive pulmonary disease, acute respiratory distress syndrome, pulmonary emphysema, pulmonary fibrosis, cystic fibrosis, silicosis, pneumonitis, acne vulgaris, atopic dermatitis, contact dermatitis, psoriasis, dermatomyositis, lichen planus, vitiligo, epidermolysis bullosa, bullous pemphigoid, hidradenitis suppurativa, harlequin fetus. Finally, in further still embodiments, the treatable disease states may include alcohol abuse, cytokine release syndrome, familial Mediterranean fever, graft-vs-host disease, mastitis, septicemia, primary sjãgren's syndrome, hyperhomo-cysteinemia, acute chest syndrome, oestrogen deficiency, painful bladder syndrome, neuropathy, allergic rhinitis, cryopyrin-associated periodic syndromes, Bechet Disease, mucocutaneous lymph node syndrome, autoimmune thrombocytopenia, deficiency of mevalonate kinase, juvenile spondyloarthropathy, and Conn Syndrome.

### DETAILED DESCRIPTION

The human body generates an inflammatory response when exposed to pathogens, tissue injury and endogenous stress factors. The inflammatory response is triggered via Pattern Recognition Receptors (PRRs). Signaling downstream to PRRs leads to expression of pro-inflammatory cytokines such as TNFα, IL-1β, IL-6, IL-18 etc. Inflammation is useful for fighting pathogens, but excessive inflammation can cause chronic or systemic inflammatory diseases where the body's immune system starts attacking its own healthy cells. However, lower levels of inflammation can result in ineffective pathogenic destruction leading to persistent infections. Hence the level of inflammation needs to be tightly regulated.

Inflammatory responses are initiated and controlled by a complex of proteins called inflammasomes, which are found in macrophages and neutrophils. When inflammasomes are overactive, diseases such as inflammatory bowel disease (IBD), irritable bowel syndrome (IBS), Crohn's disease, Alzheimer's disease, arthritis, and multiple sclerosis etc. can result. These diseases occur when the above-mentioned pro-inflammatory cytokines trigger unwanted cell death. While such cell death is a component of immune response to fight off infections, overactivity of inflammasomes trigger unwanted cell death and thus a variety of autoimmune diseases such as those mentioned above.

Without being bound by theory, it is believed that a particular family of proteins known as Apoptosis-associated speck-like protein (containing a C-terminal caspase-recruitment domain, ASC for short) interacts with procaspase-1 to, at least in part, trigger the inflammasome response that leads to cell death. (see FIG. 1). ASC regulates the assembly and activation of multiple inflammasomes. The adaptor molecule ASC plays a role in connecting stimulation and assembly of inflammasomes, by providing multiple interaction surfaces through its N-terminal PYRIN-PAAD-DAPIN domain (PYD) and a C-terminal caspase-recruitment domain (CARD). Both PYD and CARD domain belong to the death domain superfamily and possess a characteristic six-helix bundle fold. ASC brings monomers of pro-caspase-1 into proximity, which initiates caspase-1 self-cleavage and the formation of the active heterotetrametric, caspase-1. Active caspase 1 proteolytically activates several proteins, including cytokines like pro-IL-1β and pro-IL-18 (See FIG. 1), inducing their release via a non-classical secretion pathway.

Some embodiments of this invention provide a first in class pan-inflammasome inhibitor targeting ASC with a broad anti-inflammatory impact. Compounds disclosed herein inhibit ASC protein oligomerization which in turn disrupts the inflammasome assembly and thereby limits inflammation by targeting multiple inflammasome pathways. Thus, certain embodiments of the present invention have the potential to limit inflammation in various gastrointestinal and other inflammatory disorders (see FIG. 2).

While various inventive embodiments have been described and illustrated herein, those of ordinary skill in the art will readily envision a variety of other means for performing the function and/or obtaining the results and/or one or more of the advantages described herein, and each of such variations and/or modifications is deemed to be within the scope of the inventive embodiments described herein. More generally, those skilled in the art will readily appreciate that all parameters and configurations described herein are meant to be exemplary and that the actual parameters and/or configurations will depend upon the specific application or applications for which the inventive teachings is/are used.

Various inventive concepts may be embodied as one or more methods, of which examples have been provided. Unless otherwise specified, the acts performed as part of the method may be ordered in any suitable way. Accordingly, embodiments may be constructed in which acts are performed in an order different than illustrated, which may include performing some acts simultaneously, even though shown as sequential acts in illustrative embodiments.

### BRIEF DESCRIPTION OF FIGURES

Various aspects of the disclosure are set forth with particularity in the appended innovations. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic illustration that shows Inflammasome assembly activation leading to caspase-1 dependent release of pro-inflammatory cytokines, gasdermin D-mediated pyroptotic cell death, and apoptosis.

FIG. 2 is a schematic illustration of mechanism of action of compounds of the disclosure in the inhibition of inflammasome activation in inflammatory disorders.

FIG. 3 shows the graph of reduction of expression of IL-1β which is obtained by plotting percentage of cytokine inhibition (IL-1β) against the concentration of Compound 6. The graph shows that there is dose dependent decrease in IL-1β levels under in vitro conditions.

FIG. 4 shows the plot of amount of plasma IL-1β plotted against various escalating doses of Compound 6. The graph shows that there is dose dependent decrease in IL-1β levels under In vivo conditions.

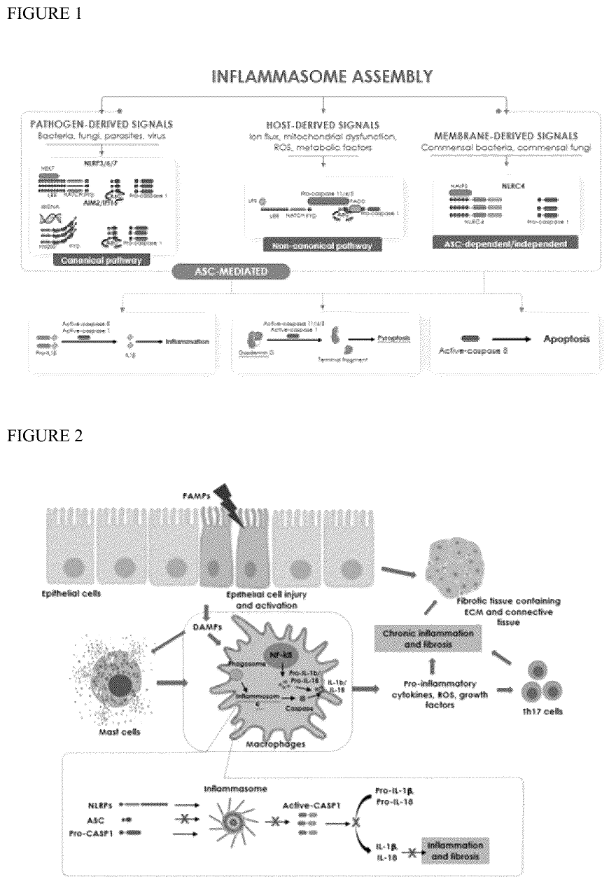

### DEFINITIONS

To facilitate understanding of the disclosure set forth herein, a number of additional terms are defined below. Unless defined otherwise, all technical and scientific terms used herein generally have the same meaning as commonly understood by one of ordinary skill in the art to which this disclosure belongs.

In this application, the use of the singular includes the plural unless specifically stated otherwise. It must be noted that, as used in the specification and the appended innovations, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, in some embodiments, ranges and amounts are expressed as “about” a particular value or range. About also includes the exact amount. Hence “about 5 μL” means “about 5 μL” and also “5 μL.” Generally, the term “about” refers to the usual experimental error range for the respective value known to persons of ordinary skill in the art.

The section headings used herein are for organizational purposes only and are not to be construed as limiting the subject matter described.

As used herein in the specification and in the claims, “or” should be understood to have the same meaning as “and/or.” For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Although the above discussion discloses various exemplary embodiments of the invention, it should be apparent that those skilled in the art can make various modifications that will achieve some of the advantages of the invention without departing from the true scope of the invention. Any references to the “invention” are intended to refer to exemplary embodiments of the invention and should not be construed to refer to all embodiments of the invention unless the context otherwise requires. The described embodiments are to be considered in all respects only as illustrative and not restrictive.

As used herein the term “ASC protein” refers to Apoptosis-associated speck-like protein containing a C-terminal caspase-recruitment domain.

“API” refers to an active pharmaceutical ingredient.

The terms “effective amount” or “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed; Gibson Ed.; CRC Press LLC: Boca Raton, Fla., 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with an acid or base. For this purpose, acids or bases, or counterions described in P. H. Stahl & C. G. Wermuth “Handbook of Pharmaceutical Salts”, Verlag Helvetica Chimica Acta, Zurich, 2002 may be employed.

As used herein, the term “IL-1β” refers to Interleukin 1 beta (IL-1β). Increased production of IL-1β causes a number of different autoinflammatory syndromes, most notably the monogenic conditions referred to as Cryopyrin-Associated Periodic Syndromes (CAPS), due to mutations in the inflammasome receptor NLRP3 which triggers processing of IL-1β.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. The term “treatment” as used herein refers to one or more of the following:
(1) prevention of a disease, for example, prevention of a disease, condition or disorder in an individual that may be predisposed to the disease, condition or disorder but does not yet experience or display the pathology or symptomatology of the disease.
(2) inhibition of a disease, for example, inhibition of a disease, condition or disorder in an individual that is experiencing or displaying the pathology or symptomatology of the disease, condition or disorder (i.e., arresting further development of the pathology and/or symptomatology); and
(3) amelioration of a disease, for example, amelioration of a disease, condition or disorder in an individual that is experiencing or displaying the pathology or symptomatology of the disease, condition or disorder (i.e., reversing the pathology and/or symptomatology).

As used in the specification and appended innovations, unless specified to the contrary, the following terms have the meaning indicated below.
“Amino” refers to the —NH2 radical.
“Cyano” refers to the —CN radical.
“Hydroxyl” refers to the —OH radical.
“Nitro” refers to the —NO2 radical.
“Oxa” refers to the —O— radical.
“Oxo” refers to the ═O radical.
“Thioxo” refers to the ═S radical.
“Imino” refers to the ═N—H radical.
“Oximo” refers to the ═N—OH radical.
“Halo” refers to fluoro (F), chloro (Cl), bromo (Br), or iodo (I).

The term “Alkyl” refers to a straight or branched hydrocarbon chain radical consisting solely of carbon and hydrogen atoms, containing no unsaturation, having from one to fifteen carbon atoms (e.g., C1-C15 alkyl). In certain embodiments, an alkyl comprises one to thirteen carbon atoms (e.g., C1-C13 alkyl). In certain embodiments, an alkyl comprises one to eight carbon atoms (e.g., C1-C8 alkyl). In other embodiments, an alkyl comprises one to five carbon atoms (e.g., C1-C5 alkyl). In other embodiments, an alkyl comprises one to four carbon atoms (e.g., C1-C4 alkyl). In other embodiments, an alkyl comprises one to three carbon atoms (e.g., C1-C3 alkyl). In other embodiments, an alkyl comprises one to two carbon atoms (e.g., C1-C2 alkyl). In other embodiments, an alkyl comprises one carbon atom (e.g., C1 alkyl). In other embodiments, an alkyl comprises five to fifteen carbon atoms (e.g., C5-C15 alkyl). In other embodiments, an alkyl comprises five to eight carbon atoms (e.g., C5-C8 alkyl). In other embodiments, an alkyl comprises two to five carbon atoms (e.g., C2-C5 alkyl). In other embodiments, an alkyl comprises three to five carbon atoms (e.g., C3-C5 alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —ORa, —SRa, —OC(O)—Ra, —N(Ra)2, —C(O)Ra, —C(O)ORa, —C(O)N(Ra)2, —N(Ra)C(O)ORf, —OC(O)—NRaRf, —N(Ra)C(O)Rf, —N(Ra)S(O)tRf (where t is 1 or 2), —S(O)tORa (where t is 1 or 2), —S(O)tRf (where t is 1 or 2), and —S(O)tN(Ra)2 (where t is 1 or 2), where each Ra is independently hydrogen, alkyl, fluoroalkyl, carbocyclyl, carbocyclylalkyl, aryl, aralkyl, heterocyclyl, heterocyclylalkyl, heteroaryl, or heteroarylalkyl, and each Rf is independently alkyl, fluoroalkyl, carbocyclyl, carbocyclylalkyl, aryl, aralkyl, heterocyclyl, heterocyclylalkyl, heteroaryl, or heteroarylalkyl.

The term “haloalkyl” refers to an alkyl, in which one or more hydrogen atoms is/are replaced with an independently selected halo.

The term “cycloalkyl” as used herein includes cyclic hydrocarbon groups having 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

The term “heterocyclyl” refers to a mono-, bi-, tri-, or polycyclic nonaromatic ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl may include multiple fused and bridged rings. Non-limiting examples of fused/bridged heterocyclyl includes: 2-azabicyclo[1.1. O]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

The term “cycloalkenyl” as used herein includes partially unsaturated cyclic hydrocarbon groups having 3 to 20 ring carbons, preferably 3 to 16 ring carbons, and more preferably 3 to 12 ring carbons or 3-10 ring carbons or 3-6 ring carbons, wherein the cycloalkenyl group may be optionally substituted. Examples of cycloalkenyl groups include, without limitation, cyclopentenyl, cyclohexenyl, cycloheptenyl, and cyclooctenyl. Cycloalkenyl groups may have any degree of saturation provided that none of the rings in the ring system are aromatic; and the cycloalkenyl group is not fully saturated overall. Cycloalkenyl may include multiple fused and/or bridged and/or spirocyclic rings.

The term “heteroaryl”, as used herein, means a mono-, bi-, tri- or polycyclic group having 5 to 20 ring atoms, alternatively 5, 6, 9, 10, or 14 ring atoms; and having 6, 10, or 14 pi electrons shared in a cyclic array; wherein at least one ring in the system is aromatic (but does not have to be a ring which contains a heteroatom, e.g. tetrahydroisoquinolinyl, e.g., tetrahydroquinolinyl), and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. The term “heterocyclyl” refers to a mono-, bi-, tri-, or polycyclic nonaromatic ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl may include multiple fused and bridged rings. Non-limiting examples of fused/bridged heterocyclyl includes: 2-azabicyclo[1.1. O]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

The term “Heteroalkyl” refers to an alkyl group as defined above in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C1-C6heteroalkyl. In some embodiments, the heteroalkyl comprises 1, 2, or 3 heteroatoms. In some embodiments, the alkyl part of the heteroalkyl radical is optionally substituted as defined for an alkyl group. Representative heteroalkyl groups include, but are not limited to —CH2NH2, —CH2NHCH3, —CH2N(CH3)2, —CH2OH, —CH2OCH3, —CH2CH2NH2, —CH2CH2NHCH3, —CH2CH2N(CH3)2, —CH2CH2OH, —CH2CH2OCH3, —CH2CH2OCH2CH2NH2, or —CH2CH2OCH2CH2OH.

The term “Heteroarylalkyl” refers to a radical of the formula -Rc-heteroaryl, where Rc is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

The term “Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. In some embodiments, the points of attachment of the alkylene chain to the rest of the molecule and to the radical group are through one carbon in the alkylene chain or through any two carbons within the chain. In certain embodiments, an alkylene comprises one to eight carbon atoms (e.g., C1-C8 alkylene). In other embodiments, an alkylene comprises one to five carbon atoms (e.g., C1-C5 alkylene). In other embodiments, an alkylene comprises one to four carbon atoms (e.g., C1-C4 alkylene). In other embodiments, an alkylene comprises one to three carbon atoms (e.g., C1-C3 alkylene). In other embodiments, an alkylene comprises one to two carbon atoms (e.g., C1-C2 alkylene). In other embodiments, an alkylene comprises one carbon atom (e.g., C1 alkylene). In other embodiments, an alkylene comprises five to eight carbon atoms (e.g., C5-C8 alkylene). In other embodiments, an alkylene comprises two to five carbon atoms (e.g., C2-C5 alkylene). In other embodiments, an alkylene comprises three to five carbon atoms (e.g., C3-C5 alkylene). Unless stated otherwise specifically in the specification, an alkylene chain is optionally substituted by one or more of the following substituents: halo, cyano, nitro, oxo, thioxo, imino, oximo, trimethylsilanyl, —ORa, —SRa, —OC(O)—Ra, —N(Ra)2, —C(O)Ra, —C(O)ORa, —C(O)N(Ra)2, —N(Ra)C(O)ORf, —OC(O)—NRaRf, —N(Ra)C(O)Rf, —N(Ra)S(O)tRf (where t is 1 or 2), —S(O)tORa (where t is 1 or 2), —S(O)tRf (where t is 1 or 2), and —S(O)tN(Ra)2 (where t is 1 or 2), where each Ra is independently hydrogen, alkyl, fluoroalkyl, carbocyclyl, carbocyclylalkyl, aryl, aralkyl, heterocyclyl, heterocyclylalkyl, heteroaryl, or heteroarylalkyl, and each Rf is independently alkyl, fluoroalkyl, carbocyclyl, carbocyclylalkyl, aryl, aralkyl, heterocyclyl, heterocyclylalkyl, heteroaryl, or heteroarylalkyl.

The term “Heterocyclylalkyl” refers to a radical of the formula -Rc-heterocyclyl where Rc is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

The “Heterocyclylalkoxy” refers to a radical bonded through an oxygen atom of the formula —O-Rc-heterocyclyl where Rc is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

The term “Amino-alkyl” refers to a radical of the formula: -alkyl-NH2.

The term “Hydroxyl-alkyl” refers to a radical of the formula: -alkyl-OH.

The term “Alkoxy” refers to a radical bonded through an oxygen atom of the formula —O-alkyl, where alkyl is an alkyl chain as defined above.

The term “Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —ORa, SRa, —OC(O)—Ra, —N(Ra)2, —C(O)Ra, —C(O)ORa, —C(O)N(Ra)2, —N(Ra)C(O)ORf, —OC(O)—NRaRf, —N(Ra)C(O)Rf, —N(Ra)S(O)tRf (where t is 1 or 2), —S(O)tORa (where t is 1 or 2), —S(O)tRf (where t is 1 or 2), and —S(O)tN(Ra)2 (where t is 1 or 2), where each Ra is independently hydrogen, alkyl, fluoroalkyl, carbocyclyl, carbocyclylalkyl, aryl, aralkyl, heterocyclyl, heterocyclylalkyl, heteroaryl, or heteroarylalkyl, and each Rf is independently alkyl, fluoroalkyl, carbocyclyl, carbocyclylalkyl, aryl, aralkyl, heterocyclyl, heterocyclylalkyl, heteroaryl, or heteroarylalkyl.

The term “Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin, and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —Rb—CN, —Rb—ORa, —Rb—OC(O)—Ra, —Rb—OC(O)—ORa, —Rb—OC(O)—N(Ra)2, —Rb—N(Ra)2, —Rb—C(O)Ra, —Rb—C(O)ORa, —Rb—C(O)N(Ra)2, —Rb—O—Re—C(O)N(Ra)2, —Rb—N(Ra)C(O)ORa, —Rb—N(Ra) C(O)Ra, —Rb—N(Ra)S(O)tRa (where t is 1 or 2), —Rb—S(O)tORa (where t is 1 or 2), —Rb—S(O)tRa (where t is 1 or 2), and —Rb—S(O)tN(Ra)2 (where t is 1 or 2), where each Ra is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl, or heteroarylalkyl, each Rb is independently a direct bond or a straight or branched alkylene or alkenylene chain, and Rc is a straight or branched alkylene or alkenylene chain, and where each of the above substituents is unsubstituted unless otherwise indicated.

The term “Aralkyl” refers to a radical of the formula -Rc-aryl where Rc is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

The term “Aralkenyl” refers to a radical of the formula -Rd-aryl where Rd is an alkenylene chain as defined above. The aryl part of the aralkenyl radical is optionally substituted as described above for an aryl group. The alkenylene chain part of the aralkenyl radical is optionally substituted as defined above for an alkenylene group.

The term “Carbocyclyl” refers to a stable non-aromatic monocyclic or polycyclic hydrocarbon radical consisting solely of carbon and hydrogen atoms, and in some embodiments, include fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond.

In some embodiments, the carbocyclyl is saturated, (i.e., containing single C—C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. In certain embodiments, a cycloalkyl comprises three to eight carbon atoms (e.g., C3-C8 cycloalkyl). In other embodiments, a cycloalkyl comprises three to seven carbon atoms (e.g., C3-C7 cycloalkyl). In other embodiments, a cycloalkyl comprises three to six carbon atoms (e.g., C3-C6 cycloalkyl). In other embodiments, a cycloalkyl comprises three to five carbon atoms (e.g., C3-C5 cycloalkyl). In other embodiments, a cycloalkyl comprises three to four carbon atoms (e.g., C3-C4 cycloalkyl). An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —CN, —Rb—ORa, —Rb—OC(O)—Ra, —Rb—OC(O)—ORa, —Rb—OC(O)—N(Ra)2, —Rb—N(Ra)2, —Rb—C(O)Ra, —Rb—C(O)ORa, —Rb—C(O)N(Ra)2, —Rb—O—Rc-C(O)N(Ra)2, —Rb—N(Ra)C(O)ORa, —Rb—N(Ra)C(O)Ra, —Rb—N(Ra)S(O)tRa (where t is 1 or 2), —Rb—S(O)tORa (where t is 1 or 2), —Rb—S(O)tRa (where t is 1 or 2), and —Rb—S(O)tN(Ra)2 (where t is 1 or 2), where each Ra is independently hydrogen, alkyl, fluoroalkyl, cycloalkyl, cycloalkylalkyl, aryl, aralkyl, heterocyclyl, heterocyclylalkyl, heteroaryl, or heteroarylalkyl, each Rb is independently a direct bond or a straight or branched alkylene or alkenylene chain, and Rc is a straight or branched alkylene or alkenylene chain, and where each of the above substituents is unsubstituted unless otherwise indicated.

The term “Carbocyclylalkyl” refers to a radical of the formula -Rc-carbocyclyl where Rc is an alkylene chain as defined above. The alkylene chain and the carbocyclyl radical are optionally substituted as defined above.

As used herein, the term “NOAEL dose” refers to the no-observed-adverse-effect level dose. It denotes the level of exposure of an organism, found by experiment or observation, at which there is no biologically or statistically significant increase in the frequency or severity of any adverse effects of the tested protocol.

As used herein the term “Inflammatory bowel disease (IBD)” refers to inflammation of the gastrointestinal (GI) tract. Prolonged inflammation results in damage to the GI tract. Crohn's disease and ulcerative colitis are considered common types of IBD. Common symptoms of IBD include persistent diarrhea, abdominal pain, rectal bleeding/bloody stools, weight loss and fatigue.

As used herein the term “Irritable bowel syndrome (IBS)” refers to a disorder that affects the large intestine. Signs and symptoms include cramping, abdominal pain, bloating, gas, and diarrhea or constipation, or both. Other symptoms that are often related include bloating, increased gas or mucus in the stool. Symptoms are treated by managing diet, lifestyle and reducing stress.

As used herein the term “Primary Sclerosing Cholangitis (PSC)” refers to a chronic liver disease in which the bile ducts inside and outside the liver become inflamed and scarred, and eventually narrowed or blocked. In some instances, bile builds up in the liver and causes further liver damage. Liver failure may occur 10-15 years after diagnosis, but this may take even longer for some PSC patients. Many people with PSC will ultimately need a liver transplant, typically about 10 years after being diagnosed with the disease.

As used herein the term “Primary biliary cholangitis (PBC)”, formerly known as primary biliary cirrhosis, is a chronic liver disease resulting from progressive destruction of the bile ducts in the liver—called the intrahepatic bile ducts.

As used herein, the term “Alcoholic hepatitis” refers to an inflammatory condition of the liver caused by heavy alcohol consumption over an extended period of time.

As used herein, the term “Alcoholic Cirrhosis” refers to a late stage of scarring (fibrosis) of the liver caused by many forms of liver diseases and conditions, such as hepatitis and chronic alcoholism.

As used herein, the term “Pancreatitis” refers to the inflammation of pancreas. It may be sudden (acute) or ongoing (chronic). The most common causes are alcohol abuse and lumps of solid material (gallstones) in the gallbladder. Pancreatitis caused by excess alcohol consumption is referred to as, alcoholic pancreatitis

As used herein, the term “Non-alcoholic steatohepatitis (NASH)” refers to an advanced form of non-alcoholic fatty liver disease (NAFLD). NAFLD is caused by buildup of fat in the liver. When this buildup causes inflammation and damage, it is known as NASH, which can lead to scarring of the liver.

As used herein the term “Celiac disease”, also known called celiac sprue or gluten-sensitive enteropathy, is an immune disorder caused by extreme sensitivity or allergic reaction to consumption of gluten protein found in wheat, barley and rye.

As used herein the term “Antiphospholipid syndrome” refers to a condition in which the immune system mistakenly creates antibodies that attack tissues in the body. These antibodies can cause blood clots to form in arteries and veins. Blood clots can form in the legs, lungs and other organs, such as the kidneys and spleen.

As used herein the term “Barrett's esophagus” is a disease that results due to repeated exposure to stomach acid. It's most often diagnosed in people with long-term gastroesophageal reflux disease (GERD). Frequent heartburn and chest pain are symptoms.

As used herein, the term “Postoperative ileus” refers to a prolonged absence of bowel function after surgical procedures, usually abdominal surgery. It is a common postoperative complication with unclear etiology and pathophysiology.

As used herein, the term “Atrophic gastritis” refers to chronic inflammation of the gastric mucosa of the stomach, leading to a loss of gastric glandular cells and their eventual replacement by intestinal and fibrous tissues. As a result, the stomach's secretion of essential substances such as hydrochloric acid, pepsin, and intrinsic factor is impaired, leading to digestive problems. The most common are vitamin B12 deficiency possibly leading to pernicious anemia; and malabsorption of iron, leading to iron deficiency anemia. It can be caused by persistent infection with Helicobacter pylori or can be autoimmune in origin. Those with autoimmune atrophic gastritis (Type A gastritis) are statistically more likely to develop gastric carcinoma, Hashimoto's thyroiditis, and achlorhydria.

As used herein, the term “Peritonitis” refers to the inflammation of the peritoneum—a silk-like membrane that lines the inner abdominal wall and covers the organs within the abdomen—that is usually due to a bacterial or fungal infection.

As used herein, the term “Diverticulitis” refers to a condition that occurs when small pouches, or sacs, form and push outward through weak spots in the wall of the colon causing pain and discomfort.

As used herein, the term “Duodenal ulcer” refers to a sore or a peptic ulcer that develops in the first part of the small intestine (duodenum).

As used herein, the term “Alveolar periostitis” refers to a condition that occurs sometimes after tooth extraction, particularly after traumatic extraction, resulting in a dry appearance of the exposed bone in the socket, due to disintegration or loss of the blood clot. It is basically a focal osteomyelitis without suppuration and is accompanied by severe pain (alveolalgia) and foul odor.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The disclosure provides compounds having formula (I):

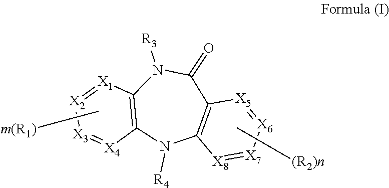

wherein R1 and R2 are each independently selected from the group consisting of hydrogen, —CO-alkyl, hydroxyl, halo, halo alkyl (C1-C6), trihalo alkyl (C1-C6), halo alkoxy, amino, C1-C6-alkyl-amino; wherein m and n are integers having each independently a value of 0, 1, 2, 3 or 4; wherein X1, X2, X3, X4, X5, X6, X7 and X8 is each independently selected from the group consisting of —CH and N;
wherein R3 is each independently selected from the group consisting of hydrogen, C1-C6 alkyl, trilhalo alkyl (C1-C6), —CO-alkyl, and —CO-haloalkyl;
wherein R4 is each independently one of hydrogen or COY with the proviso that R4 is not hydrogen when R3 is hydrogen and when X1-X8 is —CH, except that R4 and R3 may both be hydrogen when X1-X8 is N and/or R1 or R2 is a halo or

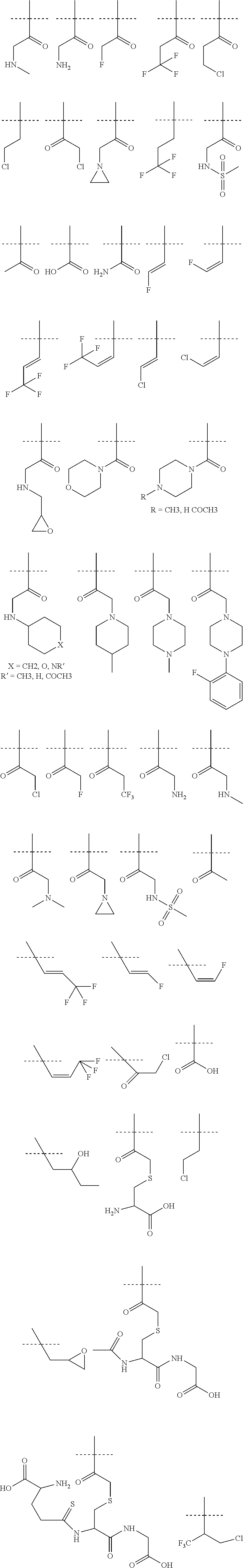

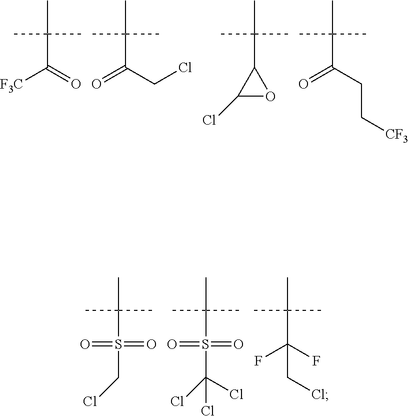

wherein Y is each independently selected from the group consisting of hydrogen, C1-C6 halo alkyl, C1-C6 halo alkoxy, C1-C6 amino alkyl, C1-C6 amino alkoxy, C3-C8 cyclo-(halo)-alkyl and wherein the alkyl or cycloalkyl group is optionally substituted with a five- or six-membered ring optionally containing at least one heteroatom selected from N, S and O, and wherein the five- or six-membered ring is optionally mono- or poly-substituted with C1-C6 alkyl, halo, C1-C6 halo alkyl, C1-C6 halo alkoxy, C1-C6 amino alkyl, C1-C6 amino alkoxy, C3-C8 cycloalkyl, C3-C8 cycloalkyl substituted with halo, amino, carboxyl or alkoxy group.

In some embodiments, in any of the aforesaid compounds, R4 is not hydrogen, m is zero and n is 1. In some embodiments, R4 is COY and Y is a substituted piperazine. In some embodiments, the R4 is COY and Y is a halo alkyl. In some embodiments, R4 is hydrogen, m is zero, n is one and R2 is halo. In some embodiments, R4 is COY and Y is a substituted piperidine.

In some embodiments, the compound is of Formula I(a)

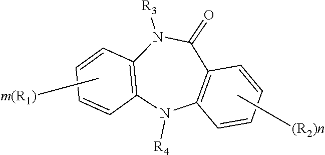

wherein R1 and R2 are each independently selected from the group consisting of hydrogen, —CO-alkyl, hydroxyl, halo, halo alkyl (C1-C6), trihalo alkyl (C1-C6), halo alkoxy, amino, C1-C6-alkyl-amino; wherein m and n are integers having each independently a value of 0, 1, 2, 3 or 4;
wherein R3 is each independently selected from the group consisting of hydrogen, C1-C6 alkyl, trilhalo alkyl (C1-C6), —CO-alkyl, and —CO-haloalkyl;
wherein R4 is each independently one of hydrogen or COY with the proviso that R4 is not hydrogen when R3 is hydrogen, except that R4 and R3 may both be hydrogen when either R1 or R2 is halo or

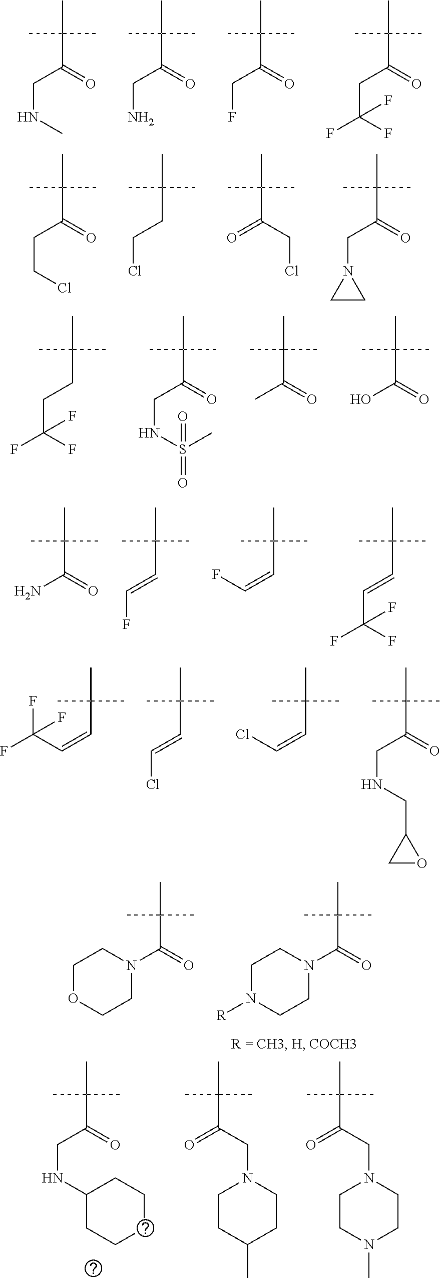

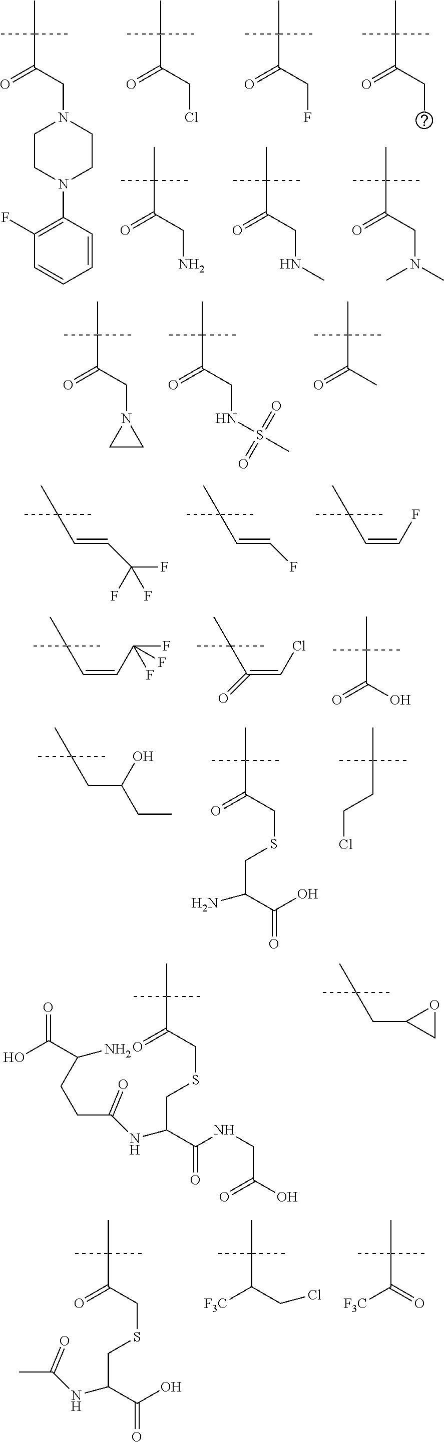

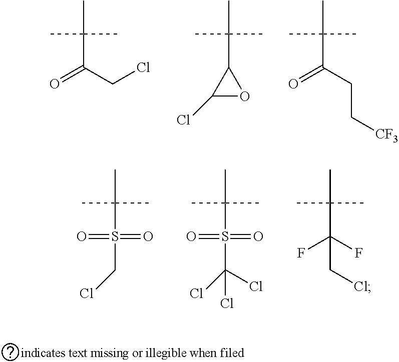

wherein Y is each independently selected from the group consisting of hydrogen, C1-C6 halo alkyl, C1-C6 halo alkoxy, C1-C6 amino alkyl, C1-C6 amino alkoxy, C3-C8 cyclo-(halo)-alkyl and wherein the alkyl or cycloalkyl group is optionally substituted with a five- or six-membered ring optionally containing at least one heteroatom selected from N, S and O, and wherein the five- or six-membered ring is optionally mono- or poly-substituted with C1-C6 alkyl, halo, C1-C6 halo alkyl, C1-C6 halo alkoxy, C1-C6 amino alkyl, C1-C6 amino alkoxy, C3-C8 cycloalkyl, C3-C8 cycloalkyl substituted with halo, amino, carboxyl or alkoxy group.

In some embodiments, the compound is of Formula I(b)

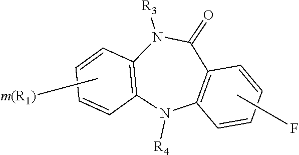

wherein R1 is each independently selected from the group consisting of hydrogen, —CO— alkyl, hydroxyl, halo, halo alkyl (C1-C6), trihalo alkyl (C1-C6), halo alkoxy, amino, C1-C6-alkyl-amino; wherein m is an integer having each independently a value of 0, 1, 2, 3 or 4; wherein R3 is each independently selected from the group consisting of hydrogen, C1-C6 alkyl, trilhalo alkyl (C1-C6), and —CO-alkyl;
wherein R4 is each independently one of hydrogen or COY or

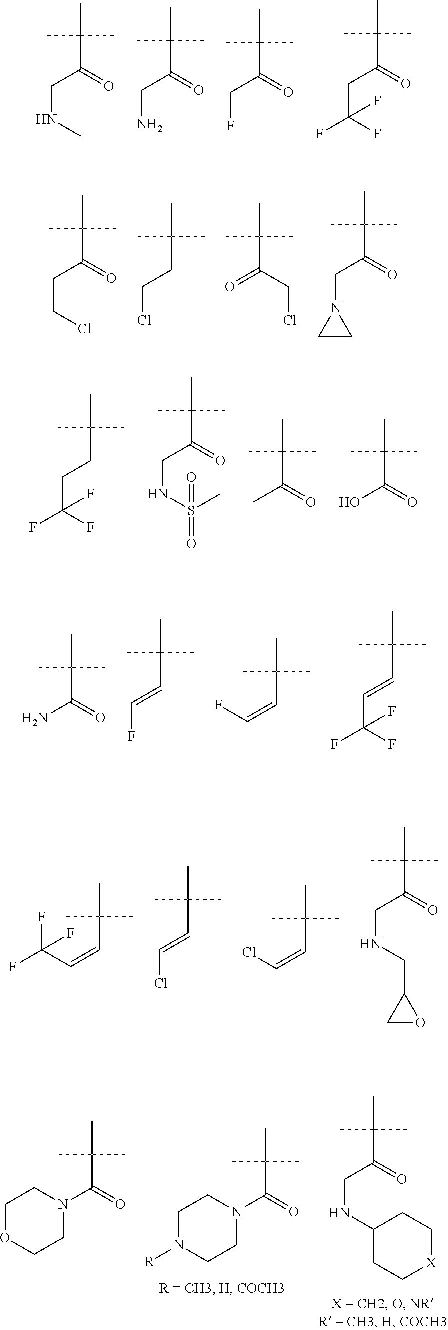

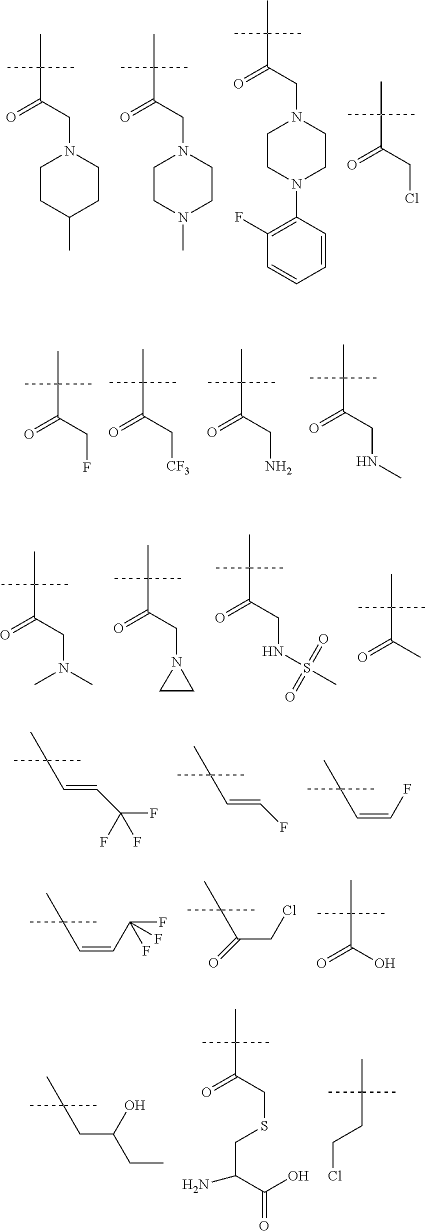

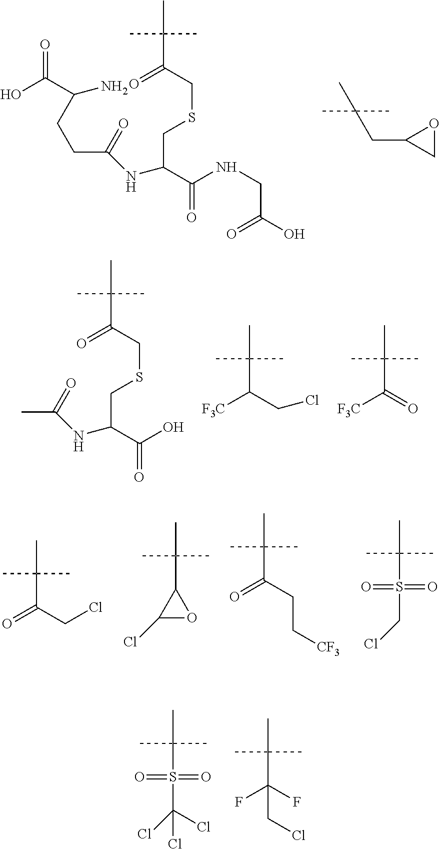

wherein Y is each independently selected from the group consisting of hydrogen, C1-C6 halo alkyl, C1-C6 halo alkoxy, C1-C6 amino alkyl, C1-C6 amino alkoxy, C3-C8 cyclo-(halo)-alkyl and wherein the alkyl or cycloalkyl group is optionally substituted with a five- or six-membered ring optionally containing at least one heteroatom selected from N, S and O, and wherein the five- or six-membered ring is optionally mono- or poly-substituted with C1-C6 alkyl, halo, C1-C6 halo alkyl, C1-C6 halo alkoxy, C1-C6 amino alkyl, C1-C6 amino alkoxy, C3-C8 cycloalkyl, C3-C8 cycloalkyl substituted with halo, amino, carboxyl or alkoxy group.

In some embodiments, the compound is a specific compound selected from the group consisting of Compounds 1-25 described above.

### Preparation of the Compounds

The compounds used in the reactions described herein are made with commercially available chemicals and/or from compounds described in the chemical literature.

In some instances, specific and analogous reactants are identified through the indices of known chemicals prepared by the Chemical Abstract Service of the American Chemical Society, which are available in most public and university libraries, as well as through on-line databases (the American Chemical Society, Washington, D.C., is contacted for more details). Chemicals that are known but not commercially available in catalogs are prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

The general reaction scheme below represents a process by which compounds of the invention can be synthesized. In addition, specific synthetic routes for Compounds 1-6 and 27-28 are set forth in the experimental examples herein, specifically, Example 1. With knowledge of the general reaction scheme below and the specific synthetic routes provided in Example 1, a person of skilled in the art could, through the exercise of ordinary skill, synthesize the additional compounds within the scope of the present specification, including Formula I, Formula Ia, and Formula Ib.

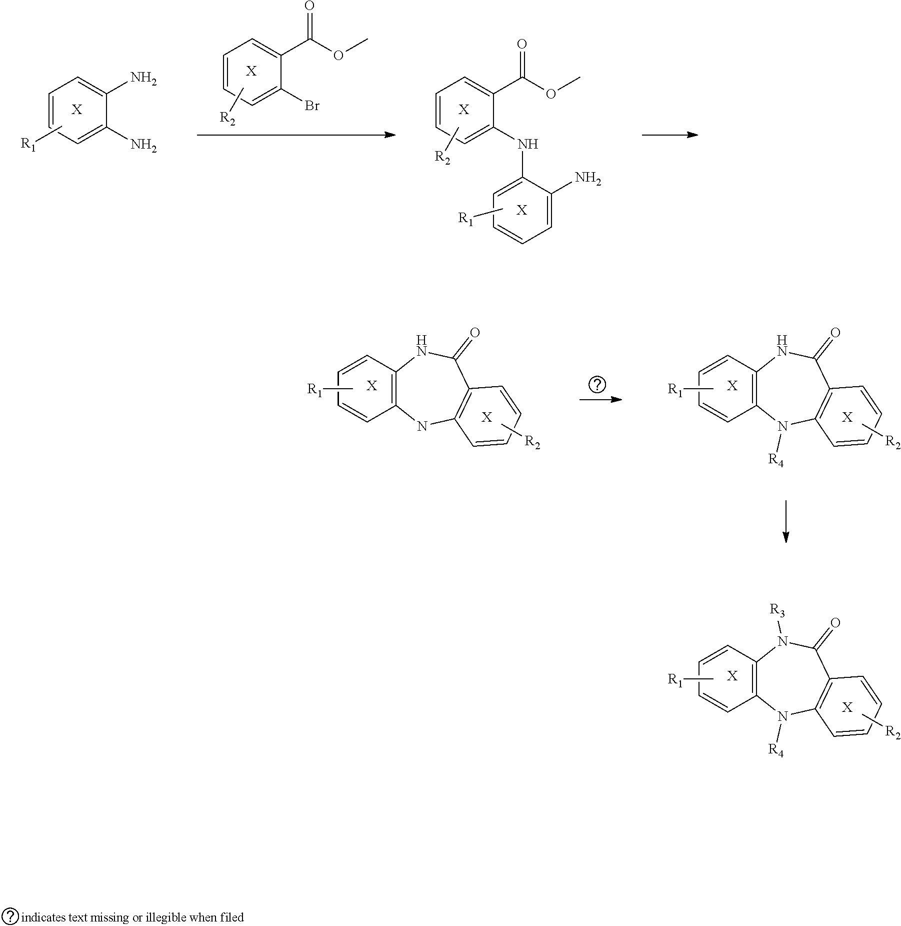

### Further Forms of Compounds

### Labeled Compounds

In some embodiments, the compounds described herein exist in their isotopically labeled forms. In some embodiments, the methods disclosed herein include methods of treating diseases by administering such isotopically labeled compounds. In some embodiments, the methods disclosed herein include methods of treating diseases by administering such isotopically labeled compounds as pharmaceutical compositions. Thus, in some embodiments, the compounds disclosed herein include isotopically labeled compounds, which are identical to those recited herein, but for the fact that one or more atoms are replaced by an atom having an atomic mass or mass number different from the atomic mass or mass number usually found in nature. In some embodiments, examples of isotopes that are incorporated into compounds of the disclosure include isotopes of hydrogen, carbon, nitrogen, oxygen, phosphorous, sulfur, fluorine, and chlorine, such as 2H, 3H, 13C, 14C, 15N, 180, 170, 31P, 32P, 35S, 18F, and 36Cl, respectively. Compounds described herein, and the metabolites, pharmaceutically acceptable salts, esters, prodrugs, solvates, hydrates, or derivatives thereof which contain the aforementioned isotopes and/or other isotopes of other atoms are within the scope of this disclosure. Certain isotopically labeled compounds, for example those into which radioactive isotopes such as 3H and 14C are incorporated, are useful in drug and/or substrate tissue distribution assays. Tritiated, i. e., 3H and carbon-14, i. e., 14C, isotopes are particularly preferred for their ease of preparation and detectability. Further, substitution with heavy isotopes such as deuterium, i.e., 2H, produces certain therapeutic advantages resulting from greater metabolic stability, for example increased in vivo half-life or reduced dosage requirements. In some embodiments, the isotopically labeled compounds, pharmaceutically acceptable salt, ester, prodrug, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of several inorganic or organic bases, and inorganic and organic acids, to form a pharmaceutically acceptable salt. In some embodiments, these salts are prepared in situ during the final isolation and purification of the compounds of the disclosure, or by separately reacting a purified compound in its free form with a suitable acid or base, and isolating the salt thus formed. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

For example, compound 6, which is a base can be reacted with a suitable acid such as hydrochloric acid to form a chloride salt of compound 6. Other suitable acids that can be used to convert the compounds of the invention such as compound 6 into pharmaceutically acceptable salts can be found in P. H. Stahl & C. G. Wermuth “Handbook of Pharmaceutical Salts”, Verlag Helvetica Chimica Acta, Zurich, 2002.

### Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. In some embodiments, solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran, or methanol. In some embodiments, the compounds provided herein exist in unsolvated as well as solvated forms. In general, the solvated forms are considered equivalent to the unsolvated forms for the purposes of the compounds and methods provided herein.

### Pharmaceutical Compositions

In certain embodiments, the compounds described herein is administered as a pure chemical. In other embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected based on a chosen route of administration. Typical administration occurs through injection or orally. Suitable forms of administration include but are not limited to oral, rectal, topical, intraperitoneal, buccal, parenteral (e.g., subcutaneous, intramuscular, intradermal, or intravenous), rectal, vaginal, or aerosol administration.

Accordingly, provided herein is a pharmaceutical composition comprising at least one compound described herein, pharmaceutically acceptable salt, hydrate, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

One embodiment provides a pharmaceutical composition comprising a pharmaceutically acceptable carrier and a compound of Formula (I) or a pharmaceutically acceptable salt or solvate thereof.

Another embodiment provides a pharmaceutical composition consisting essentially of a pharmaceutically acceptable carrier and a compound of Formula (I), or a pharmaceutically acceptable salt thereof.

In certain embodiments, the compound as described herein is substantially pure, in that it contains less than about 5%, or less than about 1%, or less than about 0.1% of other organic small molecules, such as contaminating intermediates or by-products that are created, for example, in one or more of the steps of a synthesis method.

In some instances, exemplary pharmaceutical compositions are used in the form of a pharmaceutical preparation, for example, in solid, semisolid, or liquid form, which includes one or more of a disclosed compound, as an active ingredient, in admixture with an organic or inorganic carrier or excipient suitable for external, enteral, or parenteral applications. In some embodiments, the active ingredient is compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets in some instances, the principal active ingredient is mixed with a pharmaceutical carrier, e.g., conventional tableting ingredients to form a solid preformulation composition containing a homogeneous mixture of a disclosed compound or a non-toxic pharmaceutically acceptable salt thereof. When referring to these preformulation compositions as homogeneous, it is meant that the active ingredient is dispersed evenly throughout the composition so that the composition is readily subdivided into equally effective unit dosage forms such as tablets, pills, and capsules.

In solid dosage forms for oral administration (capsules, tablets, pills, dragees, powders, granules and the like), the subject composition is mixed with one or more known pharmaceutically acceptable carriers. In the case of capsules, tablets and pills, the compositions also comprise buffering agents in some embodiments. Solid compositions of a similar type are also employed as fillers in soft and hard-filled gelatin capsules using such excipients as lactose or milk sugars, as well as high molecular weight polyethylene glycols and the like.

In some instances, a tablet is made by compression or molding, optionally with one or more accessory ingredients. Compressed tablets are prepared using binders, lubricants, inert diluents, preservatives, disintegrants and/or surface-active or dispersing agents. Molded tablets are made by molding in a suitable machine a mixture of the subject composition moistened with an inert liquid diluent. Tablets, and other solid dosage forms, such as dragees, capsules, pills and granules, are optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms contain optionally inert diluents commonly used in the art.

Suspensions, in addition to the subject composition, optionally contain known suspending agents, and mixtures thereof.

In some embodiments, formulations for rectal or vaginal administration are presented as a suppository, which are prepared by mixing a subject composition with one or more suitable non-irritating excipients or carriers comprising, which are solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component is optionally mixed under sterile conditions with a pharmaceutically acceptable carrier, and with any preservatives, buffers, or propellants which are required in some embodiments.

In some embodiments, the ointments, pastes, creams and gels contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

In some embodiments, powders and sprays contain, in addition to a subject composition, known excipients mixtures of these substances. Sprays additionally contain customary propellants, such as chlorofluorohydrocarbons and volatile unsubstituted hydrocarbons, such as butane and propane.

Compositions and compounds disclosed herein are alternatively administered by aerosol. This is accomplished by preparing an aqueous aerosol, liposomal preparation or solid particles containing the compound. A non-aqueous (e.g., fluorocarbon propellant) suspension could be used. Sonic nebulizers are used because they minimize exposing the agent to shear, which result in degradation of the compounds contained in the subject compositions in some embodiments. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which are reconstituted into sterile injectable solutions or dispersions just prior to use, which optionally contain antioxidants, buffers, bacteriostats, solutes which render the formulation isotonic with the blood of the intended recipient or suspending or thickening agents.

Also contemplated are enteral pharmaceutical formulations including a disclosed compound and an enteric material, and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0

In some embodiments, the doses of the composition comprising at least one compound as described herein differ, depending upon the patient's (e.g., human) condition, that is, stage of the disease, general health status, age, and other factors that a person skilled in the medical art will use to determine dose.

In some instances, pharmaceutical compositions are administered in a manner appropriate to the disease to be treated (or prevented) as determined by persons skilled in the medical arts. An appropriate dose and a suitable duration and frequency of administration will be determined by such factors as the condition of the patient, the type and severity of the patient's disease, the particular form of an active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. In some embodiments, the optimal dose depends upon the body mass, weight, or blood volume of the patient.

### EXAMPLES

The following examples further illustrate aspects of the present invention. However, they are in no way a limitation of the teachings or disclosure of the present invention as set forth herein.

### LIST OF ABBREVIATIONS

As used above, and throughout the disclosure, the following abbreviations, unless otherwise indicated, shall be understood to have the following meanings:
ACN or MeCN acetonitrile
Ac acetyl
BOC or Boc tert-butyl carbamate
t-Bu tert-butyl
° C. degrees Celsius
DAST—diethylaminosulfur trifluoride
DBA or dba—dibenzylideneacetone
DCE—dichloroethane (C1CH2CH2Cl)
DCM—dichloromethane (CH2Cl2)
DIPEA or DIEA—diisopropylethylamine
DMF—dimethylformamide
DMSO—dimethylsulfoxide
Dppf or dppf—1,1′-bis(diphenylphosphino)ferrocene
EA or EtOAc—ethyl acetate
Et—ethyl
EtOH—ethanol
g—gram(s)
h, hr, hrs—hour(s)
HATU 1-[bis(dimethylamino)methylene]-1H-1,2,3-triazolo[4,5-b]pyridinium 3-oxid hexafluorophosphate
HPLC—high performance liquid chromatography
Hz—hertz
LAH—lithium aluminum anhydride
LCMS—liquid chromatography mass spectrometry
m/z mass-to-charge ratio
M—molar
Me—methyl
MeOH—methanol
mg—milligram(s)
MHz—megahertz
μmol—micromole(s)
μL—microliter(s)
mL—milliliter(s)
mmol—millimole(s)
MS—mass spectroscopy
NMR—nuclear magnetic resonance
PE—petroleum ether
Ph—phenyl
prep-HPLC—preparative high pressure liquid chromatography
prep-TLC—preparative thin layer chromatography
Py—pyridine
RT—retention time
TEA—triethylamine
TFA—trifluoroacetic acid
THF—tetrahydrofuran
TLC—thin layer chromatography
XPhos—2-dicyclohexylphosphino-2′,4′,6′-triisopropylbiphenyl

### Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times were approximate and were not optimized. Column chromatography and thin layer chromatography (TLC) were performed on silica gel unless otherwise noted. In some embodiments, in case of a discrepancy between a reaction scheme and a written procedure, the written procedure should be followed.

### Example 1—Synthesis of Compounds

### Example 1a—Synthesis of Compounds 5 and 6

### Step 1: Synthesis of methyl 2-((2-aminophenyl) amino)-5-fluorobenzoate

To a stirred solution of methyl 2-bromo-5-fluorobenzoate (2 g, 8.58 mmol) in chloro benzene (20 mL) was added benzene-1,2-diamine (597 mg, 5.52 mmol) was added followed by copper powder (526 mg, 8.28 mmol). The resulting mixture was refluxed for 16 h. The progress of the reaction was monitored by TLC (30% ethyl acetate in hexane). Reaction mixture was filtered through the celite pad, filtrate was concentrated under reduced pressure. Crude product was purified by flash chromatography using 20% ethyl acetate in hexane to afford methyl 2-((2-aminophenyl) amino)-5-fluorobenzoate as brown solid (800 mg, 55.67%). LCMS: (M+H⁺=261.1)

¹H NMR (400 MHz, DMSO): δ 8.58 (s, 1H), 7.58 (dd, J=9.7, 3.0 Hz, 1H), 7.25 (td, J=8.8, 3.0 Hz, 1H), 7.00 (dd, J=16.4, 7.9 Hz, 2H), 6.81 (d, J=7.9 Hz, 1H), 6.65-6.51 (m, 2H), 4.92 (s, 2H), 3.87 (s, 3H).

### Step 2: Synthesis of 2-fluoro-5,10-dihydro-11H-dibenzo[b,e][1,4]diazepin-11-one (Compound 5)

To a stirred solution of methyl 2-((2-aminophenyl) amino)-5-fluorobenzoate (800 mg, 3.026 mmol) in ethylene glycol (10 mL) was added Tripotassium phosphate (2.5 g, 9.230 mmol), resulting mixture was heated to 100° C. for 4 h. The progress of the reaction was monitored by TLC (40% ethyl acetate in hexane). Quenched the reaction mixture with ice cold water, then extracted with Ethyl acetate, combined organic layers were dried and evaporated, crude product was purified by flash column chromatography to obtained 2-fluoro-5,10-dihydro-11H-dibenzo[b,e][1,4]diazepin-11-one as brown solid (350 mg, 25%).

¹H NMR (400 MHz, DMSO): δ 10.00 (s, 1H), 7.85 (s, 1H), 7.38 (dd, J=9.6, 3.1 Hz, 1H), 7.24 (td, J=8.5, 3.1 Hz, 1H), 7.08-6.81 (m, 5H). LCMS: (M+H⁺=229.1).

### Step 3: 5-(2-chloroacetyl)-2-fluoro-5,10-dihydro-11H-dibenzo[b,e][1,4]diazepin-11-one: (Compound 6)

To a stirred solution of 2-fluoro-5,10-dihydro-11H-dibenzo[b,e][1,4]diazepin-11-one (150 mg, 0.657 mmol) in Dichloromethane (DCM) (10 mL), 4-Dimethylaminopyridine (DMAP) was added (121 mg, 0.986 mmol) and stirred for 5 min, then 2-chloroacetyl chloride (74.2 mg, 0.657 mmol) was added at 0° C. The resulting mixture was stirred at room temperature for 4 h. The progress of the reaction was monitored by TLC (30% ethyl acetate in hexane). The Reaction mixture cooled to 0° C. quenched with Aq. sodium bicarbonate solution, then extracted with DCM. Organic layers were dried over sodium sulfate and concentrated under reduced pressure. Crude product was purified by flash column chromatography to obtained 5-(2-chloroacetyl)-2-fluoro-5,10-dihydro-11H-dibenzo [b,e][1,4]diazepin-11-one imidazo [1, 2-a]pyridine-6-carboxylate as off white solid. (120 mg, 59.92%).

LCMS (M+H⁺=305.1). ¹H NMR (400 MHz, DMSO): δ 11.03-10.65 (m, 1H), 8.00-7.61 (m, 1H), 7.65-7.12 (m, 6H), 4.47-3.92 (m, 2H).

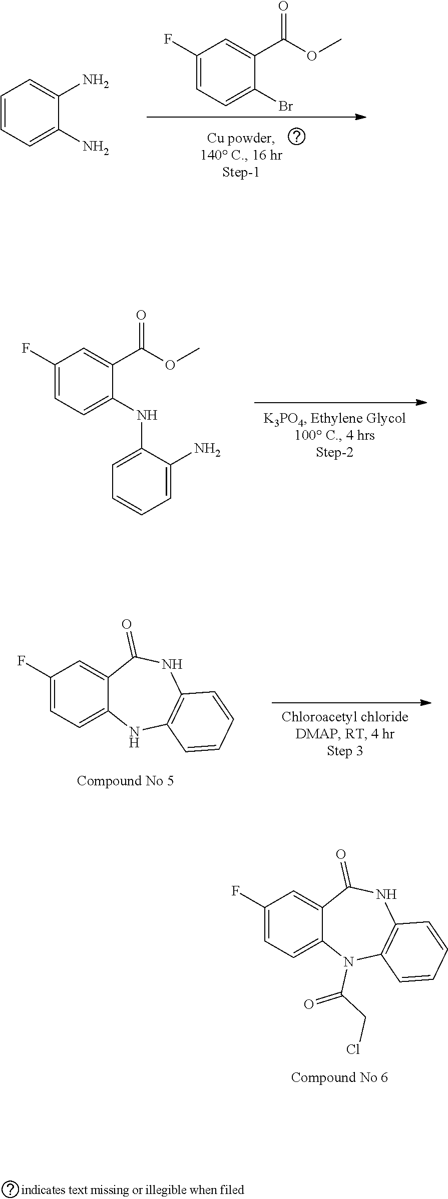 [SMILES: Nc1ccccc1N ; COC(=O)c1cc(F)ccc1Br ; COC(=O)c1cc(F)ccc1Nc1ccccc1N ; O=C1Nc2ccccc2Nc2ccc(F)cc21 ; O=C1Nc2ccccc2N(C(=O)CCl)c2ccc(F)cc21]

### Example 1b-Synthesis of Compound 1

### Synthesis of 2-fluoro-5-(2-(4-methylpiperidin-1-yl)acetyl)-5,10-dihydro-11H-dibenzo[b,e][1,4]diazepin-11-one

To a solution of 5-(2-chloroacetyl)-2-fluoro-5,10-dihydro-11H-dibenzo[b,e][1,4]diazepin-11-one (120 mg, 0.394 mmol) in acetonitrile (6 mL) was added potassium carbonate (394 mg, 0.285 mmol) stirred for 10 min followed by 4-methylpiperidine (113 mg, 0.473 mmol) was added. The resulting mixture was stirred at room temperature for 6 h. The progress of the reaction was monitored by TLC (80% ethyl acetate in hexane). Reaction mixture was concentrated under reduced pressure. Crude product was diluted with water, extracted with 5% MeOH in DCM for two times, organic layer was washed with brine solution concentrated under reduced pressure. Crude product was purified by flash column chromatography, and further purified by Prep-HPLC using formic acid buffer, concentrated under low temperature basified with aq. NaHCO₃ solution, extracted with DCM, organic layer was dried over Na₂SO₄ concentrated under reduced pressure and lyophilized to afford 2-fluoro-5-(2-(4-methylpiperidin-1-yl) acetyl)-5,10-dihydro-11H-dibenzo[b,e][1,4]diazepin-11-one as white solid. (47.3 mg, 13.4%).

¹H NMR (400 MHz, CD₃OD_SPE): δ 7.91-6.99 (m, 7H), 3.29-2.99 (m, 2H), 2.92-2.39 (m, 2H), 2.10-1.85 (m, 2H), 1.67-0.98 (m, 5H), 0.90 (s, 3H). LCMS (M+H⁺=368.1).

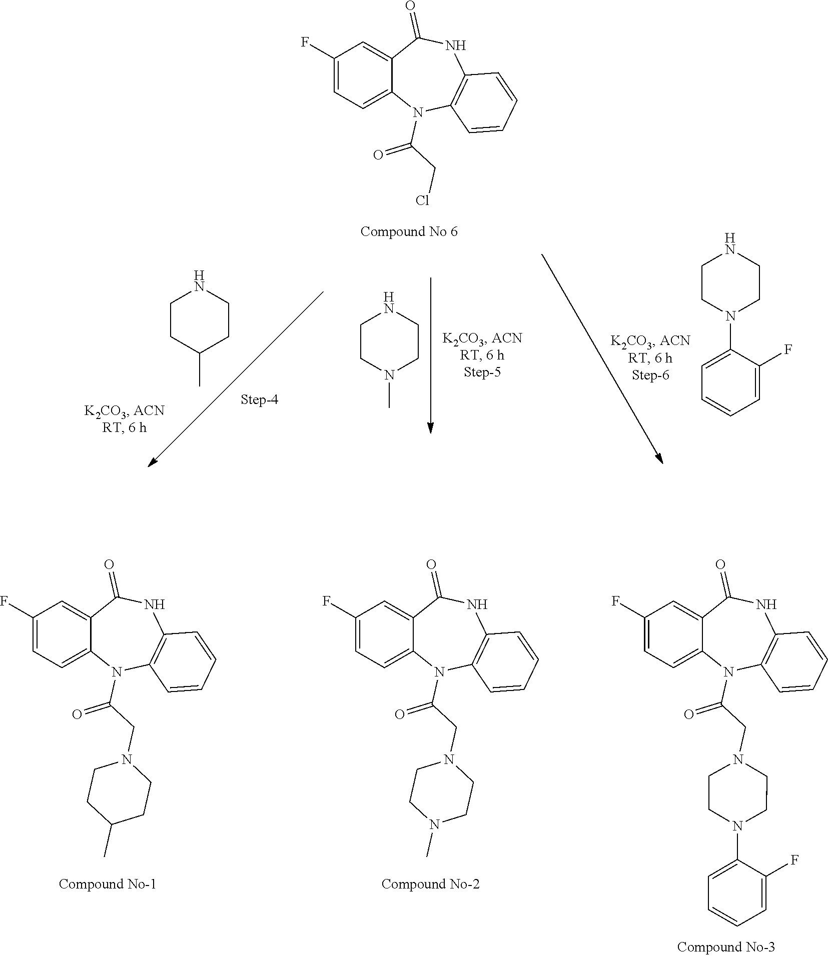

### Example 1c-Synthesis of Compound 2

### 2-fluoro-5-(2-(4-methylpiperazin-1-yl)acetyl)-5,10-dihydro-11H-dibenzo[b,e][1,4]diazepin-11-one

To a solution of 5-(2-chloroacetyl)-2-fluoro-5,10-dihydro-11H-dibenzo[b,e][1,4]diazepin-11-one (120 mg, 0.394 mmol) in acetonitrile (6 mL) was added potassium carbonate (394 mg, 0.285 mmol) stirred for 10 min followed by 1-methylpiperazine (143 mg, 0.592 mmol) was added. The resulting mixture was stirred at room temperature for 6 h. Monitored the progress of the reaction by TLC (10% MeOH in DCM). Reaction mixture was concentrated under reduced pressure. The residue was diluted with 10% MeOH in DCM, organic was washed with brine solution concentrated under reduced pressure. Crude product was purified by flash column chromatography, further purified by Prep-HPLC using formic acid buffer, concentrated under low temperature basified with aq. NaHCO₃ solution, extracted with DCM, organic layer was dried over Na₂SO₄ concentrated under reduced pressure and lyophilized to afford 2-fluoro-5-(2-(4-methylpiperazin-1-yl) acetyl)-5,10-dihydro-11H-dibenzo[b,e][1,4]diazepin-11-one as white solid. (31 mg, 8.85%).

¹H NMR (400 MHz, CD₃OD_SPE): δ 7.71-7.35 (m, 5H), 7.37-7.15 (m, 2H), 3.16 (dt, J=18.7, 15.1 Hz, 2H), 2.67-2.26 (m, 11H). LCMS (M+H⁺=369.1).

### Example 1d—Synthesis of Compound 3

### 2-fluoro-5-(2-(4-(2-fluorophenyl)piperazin-1-yl)acetyl)-5,10-dihydro-11H-dibenzo[b,e][1,4]diazepin-11-one

To a solution of 5-(2-chloroacetyl)-2-fluoro-5,10-dihydro-11H-dibenzo[b,e][1,4]diazepin-11-one (120 mg, 0.394 mmol) in acetonitrile (6 mL) was added potassium carbonate (394 mg, 0.285 mmol) stirred for 10 min followed by 1-(2-fluorophenyl)piperazine (206 mg, 0.473 mmol) was added. The resulting mixture was stirred at room temperature for 6 h. The progress of the reaction was monitored by TLC (10% MeOH in DCM). Reaction mixture was concentrated under reduced pressure. The residue was diluted with 10% MeOH in DCM, organic was washed with brine solution concentrated under reduced pressure. Crude product was purified by flash column chromatography, further purified by Prep-HPLC using formic acid buffer, concentrated under low temperature basified with aq. NaHCO₃ solution, extracted with DCM, organic layer was dried over Na₂SO₄ concentrated under reduced pressure and lyophilized to afford 2-fluoro-5-(2-(4-(2-fluorophenyl)piperazin-1-yl)acetyl)-5,10-dihydro-11H-dibenzo[b,e][1,4]diazepin-11-one as off white solid. (48.3 mg, 11.32%).

¹H NMR (400 MHz, MeOD): δ 7.81-7.15 (m, 7H), 7.17-6.80 (m, 4H), 3.44 (dd, J=32.6, 17.9 Hz, 0.4H), 3.30-3.08 (m, 2H), 3.01 (s, 4H), 2.69-2.28 (m, 4H). LCMS (M+H⁺=449.1).

### Example 1e-Synthesis of Compound 4

### Step 1: 5-(2-chloroacetyl)-5,10-dihydro-11H-dibenzo[b,e][1,4]diazepin-11-one

To a stirred solution of 5,10-dihydro-11H-dibenzo[b,e][1,4]diazepin-11-one (200 mg, 0.952 mmol) in DCM (20 mL) was added DMAP (174 mg, 1.428 mmol) it was stirred for 5 min, then added 2-chloroacetyl chloride (106 mg, 0.952 mmol) at 0° C. The resulting mixture was stirred at room temperature for 4 h. The progress of the reaction was monitored by TLC (30% ethyl acetate in hexane). The Reaction mixture cooled to 0° C. quenched with aq sat sodium bicarbonate solution, then extracted with DCM. Organic was concentrated under reduced pressure. Crude product was purified by flash column chromatography to 5-(2-chloroacetyl)-5,10-dihydro-11H-dibenzo[b,e][1,4]diazepin-11-one as off white solid. (120 mg, 30.32%). LCMS (M+H⁺=287.1)

¹H NMR (400 MHz, T MeOD): δ 8.11-7.11 (m, 8H), 4.36-3.97 (m, 2H).

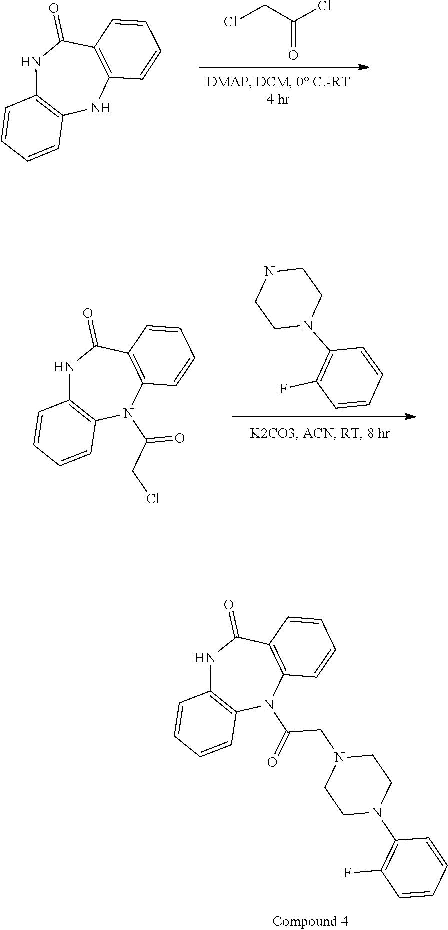

### Step 2: Synthesis of 5-(2-(4-(2-fluorophenyl)piperazin-1-yl)acetyl)-5,10-dihydro-11H-dibenzo[b,e][1,4]diazepin-11-one (Compound 4)

To a solution of 5-(2-chloroacetyl)-5,10-dihydro-11H-dibenzo[b,e][1,4]diazepin-11-one (100 mg, 0.349 mmol) in acetonitrile (5 mL) was added potassium carbonate (144 mg, 1.047 mmol) stirred for 10 min followed by 1-(2-fluorophenyl)piperazine (62 mg, 0.349 mmol) was added. The resulting mixture was stirred at room temperature for 6 h. The progress of the reaction was monitored by TLC (80% ethyl acetate in hexane). Reaction mixture was concentrated under reduced pressure. Crude product was diluted with 5% MeOH in DCM, organic was washed with brine solution concentrated under reduced pressure. Crude product was purified by flash column chromatography, Then crude was further purified by Prep-HPLC using formic acid buffer, concentrated under low temperature basified with aq. NaHCO₃ solution, extracted with DCM, organic layer was dried over Na₂SO₄ concentrated under reduced pressure and lyophilized to afford 5-(2-(4-(2-fluorophenyl)piperazin-1-yl)acetyl)-5,10-dihydro-11H-dibenzo[b,e][1,4]diazepin-11-one. (18.1 mg, 12%).

¹H NMR (400 MHz, MeOD): δ 8.01-7.76 (m, 1H), 7.76-7.55 (m, 2H), 7.49 (t, J=15.5 Hz, 2H), 7.45-7.20 (m, 3H), 7.14-6.88 (m, 4H), 3.50-3.35 (m, 0.8H), 3.32-3.11 (m, 1.6H), 2.95 (d, J=31.2 Hz, 4H), 2.68-2.22 (m, 4H). LCMS (M+H⁺=431.1)

### Example 1f-Synthesis of Compound 27

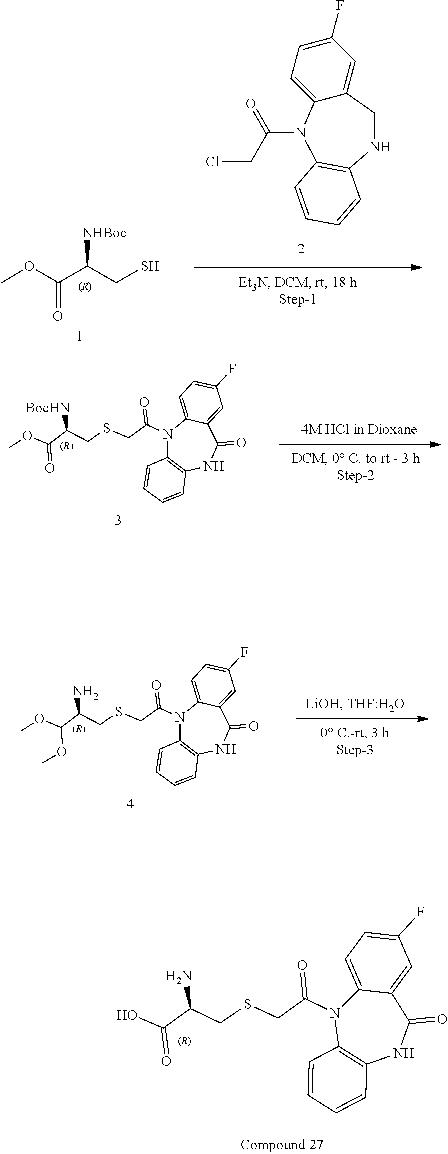

### Step-1: Synthesis of methyl N-(tert-butoxycarbonyl)-S-(2-(2-fluoro-11-oxo-10,11-dihydro-5H-dibenzo[b,e][1,4]diazepin-5-yl)-2-oxoethyl)-L-cysteinate

To a stirred solution of methyl (tert-butoxycarbonyl)-L-cysteinate (0.1 g, 0.425 mmol) in dichloromethane (10 mL) were added triethylamine (64.5 mg, 0.637 mmol) and 5-(2-chloroacetyl)-2-fluoro-5,10-dihydro-11H-dibenzo[b,e][1,4]diazepin-11-one (117 mg, 0.382 mmol) at 0° C. and Stirred the reaction mixture at RT for 12 h. Progress of reaction was monitored by TLC. After completion of starting material, reaction mixture was diluted with water and extracted with EtOAc, combined organic layer was dried over Na₂SO₄ and concentrated under reduced pressure. The crude compound was purified by combi-flash column chromatography eluted with 0-25% EtOAc/Hex to obtain methyl N-(tert-butoxycarbonyl)-S-(2-(2-fluoro-11-oxo-10,11-dihydro-5H-dibenzo[b,e][1,4]diazepin-5-yl)-2-oxoethyl)-L-cysteinate (150 mg, 70.93% yield) as off white solid.

1H-NMR (400 MHz, DMSO-d6): δ10.8 (d, J=31.6 Hz, 1H), 7.84-7.63 (m, 1H), 7.62-7.48 (m, 3H), 7.47-7.37 (m, J H), 7.33-7.18 (m, 3H), 4.2-4.02 (m, J H), 3.60 (s, 3H), 3.48-3.28 (m, 2H), 2.92-2.6 (m, 2H), 1.36 (s, 9H)

### Step-2: Synthesis of methyl S-(2-(2-fluoro-11-oxo-10,11-dihydro-5H-dibenzo[b,e][1,4]diazepin-5-yl)-2-oxoethyl)-L-cysteinate

To a stirred solution of methyl N-(tert-butoxycarbonyl)-S-(2-(2-fluoro-11-oxo-10,11-dihydro-5H-dibenzo[b,e][1,4]diazepin-5-yl)-2-oxoethyl)-L-cysteinate (110 mg, 0.218 μmol) in dichloromethane (4 mL) was added trifluoroacetic acid (0.5 mL) at 0° C. and stirred the reaction mixture at 0-RT for 3 h. Progress of reaction was monitored by TLC. After completion of reaction, reaction mixture was concentrated under reduced pressure, co-distilled with DCM, triturated with diethyl ether to obtain methyl S-(2-(2-fluoro-11-oxo-10,11-dihydro-5H-dibenzo[b,e][1,4]diazepin-5-yl)-2-oxoethyl)-L-cysteinate (80 mg, crude) as off-white solid. The crude compound was taken to next step as such.

1H-NMR (400 MHz, DMSO-d6): δ10.82 (d, J=32.4 Hz, JH), 8.44 (brs, 3H), 7.88-7.67 (m, JH), 7.62-7.46 (m, 3H), 7.45-7.38 (m, J H), 7.37-7.2 (m, 3H), 4.29 (d, J=6 Hz, J H), 3.8-3.52 (m, 4H), 3.33-3.22 (m, JH), 3.15-2.85 (m, 2H)

LCMS: m/z (m+H, 404.32, 426 (Na adduct)

### Step-3: Synthesis of S-(2-(2-fluoro-11-oxo-10,11-dihydro-5H-dibenzo[b,e][1,4]diazepin-5-yl)-2-oxoethyl)-L-cysteine

To a stirred solution of methyl S-(2-(2-fluoro-11-oxo-10,11-dihydro-5H-dibenzo[b,e][1,4]diazepin-5-yl)-2-oxoethyl)-L-cysteinate (120 mg, 0.297 mmol) in tetrahydrofuran (3 mL) and water (3 mL) was added lithium hydroxide (14.2 mg, 0.595 mmol) at 0° C. and stirred the reaction mixture at RT for 3 h. Progress of reaction was monitor by TLC. Reaction mixture was concentrated under reduced pressure. The crude compound was purified by prep-HPLC to obtain S-(2-(2-fluoro-11-oxo-10,11-dihydro-5H-dibenzo[b,e][1,4]diazepin-5-yl)-2-oxoethyl)-L-cysteine (32 mg, 27.58% yield) as white solid.

1H-NMR (400 MHz, D2O): δ7.7-7.58 (m, 1H), 7.57-7.23 (m, 5H), 7.28 (d, J=7.6 Hz, JH), 3.9-3.78 (m, JH), 3.6-3.4 (m, 2H), 3.1-2.8 (m, 2H)

LCMS: m/z (m+H, 390.44)

### Example 1g-Synthesis of Compound 28

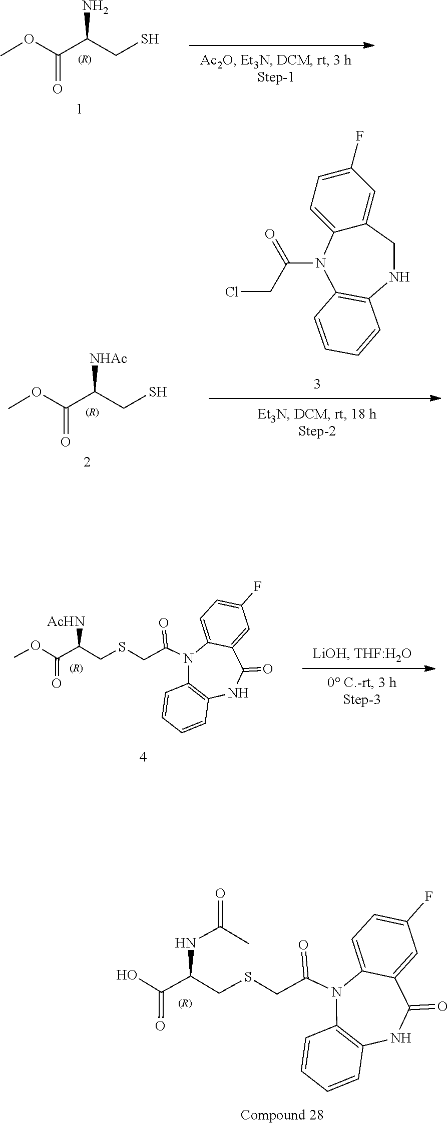

### Step-1: Synthesis of methyl acetyl-L-cysteinate

To a stirred solution of hydrogen chloride-methyl (R)-2-amino-3-mercaptopropionate (1/1) (2 g, 11.7 mmol) in dichloromethane (50 mL) were added triethylamine (3.6 mL, 25.63 mmol) and acetic anhydride (1.3 g, 12.817 mmol) dropwise at 0° C. The reaction mixture was stirred at RT for 3 h. Progress of reaction was monitored by TLC. After completion of reaction, the reaction mixture was diluted with water (100 mL) and extracted with DCM (3×100 mL). The organic layer was dried over sodium sulfate and concentrated under reduced pressure. The crude compound was purified by combi-flash chromatography, eluted with (0-100% in DCM in hexane) to afford methyl acetyl-L-cysteinate (0.3 g, yield-11.45%) as white solid.

1H-NMR (400 MHz, CDCL3): δ 6.36 (s, 1H), 4.95-4.87 (m, 1H), 3.8 (s, 3H), 3.03-2.99 (m, 2H), 2.07 (s, 3H), 1.32 (t, J=8.8 Hz, 1H).

### Step-2: Synthesis of methyl N-acetyl-S-(2-(2-fluoro-11-oxo-10,11-dihydro-5H-dibenzo[b,e][1,4]diazepin-5-yl)-2-oxoethyl)-L-cysteinate

To a stirred solution of methyl acetyl-L-cysteinate (64 mg, 0.361 mmol) in dichloromethane (5 mL) were added triethylamine (66 mg, 0.657 mmol) followed by 5-(2-chloroacetyl)-2-fluoro-5,10-dihydro-11H-dibenzo[b,e][1,4]diazepin-11-one (100 mg, 0.328 mmol) at 0° C., The reaction mixture was stirred at RT for 18 h. Progress of reaction was monitor by TLC. After completion of reaction, quenched with water and extracted with EtOAc, combined organic layer was dried over Na2SO4 and concentrated under reduced pressure. The crude compound was purified by combi-flash column chromatography eluted with (0-25% EtOAc/Hexane) to obtain methyl N-acetyl-S-(2-(2-fluoro-11-oxo-10,11-dihydro-5H-dibenzo[b,e][1,4]diazepin-5-yl)-2-oxoethyl)-L-cysteinate (130 mg, 89% yield) as off white solid.

1H-NMR (400 MHz, DMSO-d6): δ10.81 (d, 1H), 8.4-8.25 (m, 1H), 7.85-7.64 (m, 1H), 7.6-7.49 (m, 3H), 7.48-7.18 (m, 3H), 4.5-4.3 (m, 1H), 3.61 (s, 3H), 3.52-3.2 (m, 2H), 3.0-2.7 (m, 2H), 1.82 (s, 3H)

### Step-3: Synthesis of N-acetyl-S-(2-(2-fluoro-11-oxo-10,11-dihydro-5H-dibenzo[b,e][1,4]diazepin-5-yl)-2-oxoethyl)-L-cysteine

To a stirred solution of methyl N-acetyl-S-(2-(2-fluoro-11-oxo-10,11-dihydro-5H-dibenzo[b,e][1,4]diazepin-5-yl)-2-oxoethyl)-L-cysteinate (120 mg, 0.269 mmol) in tetrahydrofuran (3 mL) and water (3 mL) was added lithium hydroxide (14.2 mg, 0.595 mmol) at 0° C. The reaction mixture was stirred at 0-RT for 3 h. Progress of reaction was monitored by TLC. The reaction mixture was concentrated to remove THF, reaction mixture diluted with water, extracted with EtOAc. The aqueous layer was acidified with 1N HCl, extracted with EtOAc. Organic layer was dried over Na2SO4 and concentrated under reduced pressure. The crude compound was purified by prep-HPLC to obtain N-acetyl-S-(2-(2-fluoro-11-oxo-10,11-dihydro-5H-dibenzo[b,e][1,4]diazepin-5-yl)-2-oxoethyl)-L-cysteine (50 mg, 51.8% yield) as off-white solid.

1H-NMR (400 MHz, DMSO-d6): δ12.79 (brs, 1H), 10.8 (d, 1H), 8.24-8.1 (m, 1H), 7.9-7.6 (m, 1H), 7.6-7.3 (m, 4H), 7.29-7.15 (m, 2H), 4.42-4.2 (m, 1H), 3.5-3.2 (m, 2H), 3-2.62 (m, 2H), 1.81 (s, 3H)

LCMS: (m/z, m+H, 432.1)

Synthesis of novel compounds with modification in core structure can be achieved using two different strategies. Firstly, Chemical modification of Compound 5/6 by substitution/reaction with suitable reagent to introduce the desired modification in the core structure. These modifications include functional group modification, ring substitution. Secondly, synthesis with the commercially available key starting material that already possesses the necessary substitution or functional group to synthesize the core structure. The choice of reagents, reaction condition and protecting group can play a crucial role in the successfully execution of these strategies. To the modified core structure side chain can be incorporated using the same synthetic protocol as disclosed for compound 6.

### Example 2—In Vitro Inhibition of Production of IL-16

The following example shows the efficacy of the compounds of the disclosure to inhibit the production of cytokines such as Interleukin-1p in a dose dependent manner under in vitro conditions. The procedures detailed here can be utilized for testing all compounds of the disclosure.

Human monocytic THP-1 cells were treated with phorbol 12-myristate 13 acetate (PMA) (100 nM) overnight to differentiate monocytic cells into macrophages. Then, after 24 hours of rest, the differentiated cells were incubated with test compounds for 12 hours at concentrations 0.01, 0.1, 1, 10, 30, and 50 μM. Cells were then washed once with PBS and primed with E. coli LPS (0.1 μg/ml) for 4 h followed by stimulation with ATP (5 μM) for an additional 30 min. The cell culture supernatants were then collected and OD values for IL-1β levels were measured by ELISA. The standard curve (OD VS concentration) for IL-1β was also plotted and used to determine the IL-1β concentration for the OD values. The percentage of inhibition of IL-1β was calculated using the formula:

$$\frac{( IL - 1 β concentration with LPS + ATP - IL - 1 β concentration for the sample ] * 100}{( IL - 1 β concentration with LPS + ATP - IL - 1 β concentration with DMSO )}$$

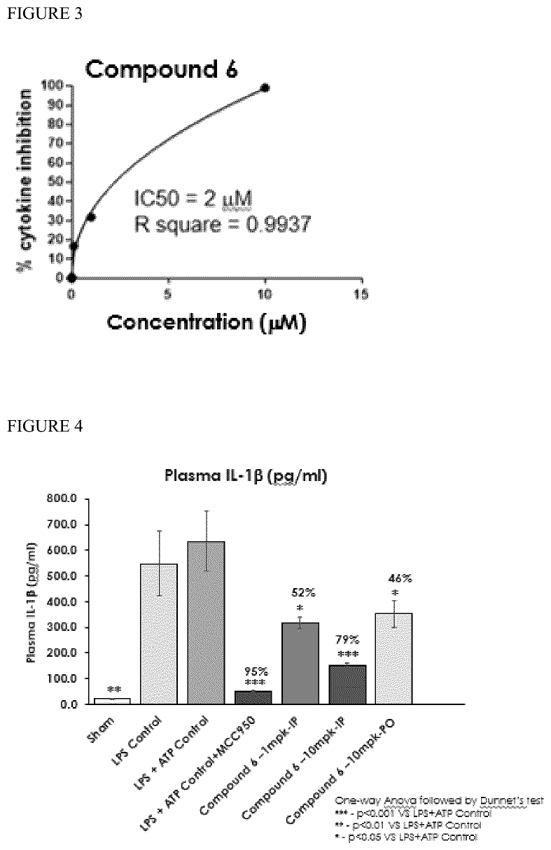

The percentage IL-1β inhibition at different compound concentrations were plotted in a sigmoid curve and IC50 values were interpolated in Graphpad Prism. Cell death was evaluated by propidium iodide staining and OD was measured at 490 nm. Cell viability was estimated with respect to DMSO control. FIG. 3 displays the IC50 values of a representative compound—Compound 6 and it had 99% cell viability at 10 μM indicating that it is not toxic to cells at the concentration range tested.

### Example 3—In Vivo Inhibition of Production of IL-1B

The following example shows the efficacy of the compounds of the disclosure to inhibit the production of cytokines such as Interleukin-1β in a dose dependent manner under in vivo conditions. The procedures detailed here can be utilized for testing all compounds of the disclosure. The example utilizes LPS+ATP-induced model of inflammasome activation in 8-10 weeks old male Balb/c mice for in vivo testing.

8-10 weeks old male Balb/c mice were challenged by intraperitoneal (i.p.) injection of 200 μg/Kg (0.2 μg/g) LPS at t=0 hr, followed by i.p. injection of 5 mM solution of ATP at t=2 hr (2 h after LPS challenge). The compound of disclosure being tested for inhibitory activity (such as Compound 6) was administered both via intraperitoneal and oral route at −1 hr. Different doses of the compound of the disclosure were given to the mice after LPS priming as shown below.

MCC950, a specific small molecule inhibitor of NLRP3 inflammasome was used as a positive control to enable comparison of inhibitory activity of the compounds of the disclosure along with a known inhibitor. 2.5 hours after the LPS priming (or 0.5 hours after ATP administration), blood was collected via retro-orbital bleeding in heparinized blood collection tubes. Blood was centrifuged for 5 minutes at 10,000 rpm in a refrigerated centrifuge to obtain plasma. Separated plasma was used for cytokine assessment by ELISA. The standard curve (OD VS concentration) for IL-1β was also plotted and used to determine the IL-1β concentration for the OD values. The percentage of inhibition of IL-1β was calculated using the formula:

$$\frac{( IL - 1 β concentration in LPS + ATP control - IL - 1 β concentration for the sample ] * 100}{( IL - 1 β concentration in LPS + ATP control - IL - 1 β concentration in sham control )}$$

The following groups were evaluated and IL-β inhibition was observed:

| | | Route of | | % IL-1 β |
|---|---|---|---|---|
| Groups | Dose | administration | N | inhibition |
| Sham control | — | — | 6 | — |
| LPS control | — | — | 6 | — |
| LPS + ATP | — | — | 6 | — |
| MCC950 | 20 mg/kg | Intraperitoneal | 6 | 95% |
| Compound 6 | 1 mg/kg | Intraperitoneal | 6 | 52% |
| Compound 6 | 10 mg/kg | Intraperitoneal | 6 | 79% |
| Compound 6 | 10 mg/kg | Oral | 6 | 46% |

FIG. 4 shows a dose dependent reduction of IL-β for Compound 6 in an in vivo mouse model. It is interesting to note that this compound exhibited 79% inhibition of IL-1β at 10 mg/kg, i.p. and 46% inhibition of IL-1β tested at 10 mg/kg, dosed orally. MCC950 was used as a tool compound in this assay.

Thus, the compound of the disclosure showed significant inhibition of IL-β generation in LPS+ATP-induced mice model of inflammasome activation

### Example 4—Analysis of Absorption. Distribution, Metabolism, and Excretion (ADME) and Pharmacokinetics (PK) Parameters

ADME describes the absorption, distribution, metabolism, and excretion of drugs in the body. The following example describes the process of evaluation of ADME parameters of compounds of the disclosure.

### Solubility Analysis

Seven level calibration standards (i.e., 1, 5, 10, 50, 100, 200 and 300 μM) of the test compound were prepared from 20 mM primary stock solution in DMSO. An aliquot of 198 μL of PBS (pH-7.4) was dispensed into duplicate wells of a multiscreen solubility filter plate. Subsequently, 2 μL of test compound solution from 20 mM primary stock solution was added to give a final concentration of 200 μM, the plate was covered and shaken at 150 rotations per minute for 90 minutes. At the end of 90 minutes, samples were filtered using MultiScreen HTS vacuum Manifold assembly and the filtrates were collected into the acceptor plate. An aliquot of 150 μL of filtrate from the above 96-well acceptor plate were transferred into HPLC vials and analyzed by HPLC-PDA. Solubility was determined by comparing the absorbance of test/reference compound against the respective DMSO calibration curve. The compound Compound 6 was tested for solubility in phosphate buffer saline (pH-7.4) by kinetic method and compound was highly soluble with a solubility of >200 μM.

### Metabolic Stability Assay in Mice, Rat and Human Liver Microsomes

The assay was performed in duplicate, and the final test concentration was 1 μM. The incubations were carried out for 45 min with intermediate time points of 0-, 5-, 15-, and 30-min. Verapamil was used as a reference standard for this experiment. The concentration of verapamil was 1 μM. For the microsomal stability experiment, vials containing the microsomes were thawed an ice bath. 33 μL microsomes (20 mg/mL) was suspended in 1165.7 μL of 100 mM potassium phosphate buffer (pH 7.4) in the propylene tube labeled as incubation mixture. The control as well as test compounds will have similar set of incubation mixture tubes. 1.1 μL of Compound 6 or verapamil (1 mM) was added to above incubation mixtures to give a working concentration of 1.1 μM respectively. 180 μL aliquot from incubation mixtures was transferred to 6 tubes labeled T_{Control}, T₀, T₅, T₁₅, T₃₀ and T₄₅.

All the tubes were pre-incubated at 37 t 1° C. for 5 min in a shaking water bath. Tubes of NADPH solution (10 mM) were similarly pre-incubated in similar conditions. After pre-incubation, 20 μL NADPH solution (10 mM) was added to the T₀, T₅, T₁₅, T₃₀, and T₄₅ tubes, and 20 μL of buffer was added to the T_{Control} tube which makes the final concentration to 1 μM respectively. T₀ was immediately quenched with 200 μL of quenching solution containing warfarin as internal standard (IS). Similarly, at the end of the incubation period (5, 15, 30, and 45 minutes) of respective tubes, 200 μL of quenching solution containing warfarin as internal standard (IS) was added to each tube to stop the reaction. Resulting samples were centrifuged at 3220 g (relative centrifugal force) for 20 min. Supernatant (200 μL) from each reaction tube was taken for LC-MS/MS analysis.

Calculations were done as follows:

$$\begin{matrix}\% remaining = 100 \times ( PAR at appointed incubation time / PAR at {T}_{0} time ) \\ Elimination rate constant ( k ) = ( - gradient ) \\ Half - life ( t 1 / 2 ) = 0.693 / k \\ V ( μ L / mg ) = volume of incubation ( μ L ) / protein in the incubation ( mg ) \\ Microsomal Intrinsic Clearance ( mC Lint ) ( μ L / min / mg of protein ) = ( V * 0.693 ) / {t}_{1 / 2} \\ where PAR is the peak area ratio of analyte versus internal standard ( IS . )\end{matrix}$$

The stability of Compound 6 when incubated in mice, rat, and human liver microsomes along with reference standard verapamil is tabulated below:

| Compound | Assay | % rem @ | % rem @ | % rem @ | % rem @ | t_{1/2} | mCLint |
|---|---|---|---|---|---|---|---|
| ID | matrix | 5 min | 15 min | 30 min | 45 min | (min) | (μL/min/mg) |
| Compound 6 | HLM | 93.5 | 92.9 | 91.3 | 67.8 | 96.3 | 14.4 |
| Verapamil | | 43.3 | 18.8 | 6.68 | 3.69 | 9.78 | 142 |
| Compound 6 | MLM | ~100 | ~100 | 79.3 | 59.8 | 57.0 | 24.3 |
| Verapamil | | 13.6 | 2.76 | 0.78 | 0.29 | 5.83 | 238 |
| Compound 6 | RLM | 98.7 | 83.1 | 75.3 | 74.0 | 94.7 | 14.6 |
| Verapamil | | 23.3 | 6.04 | 1.07 | 0.43 | 5.95 | 233 |

The reference standard, verapamil showed high degree of metabolism by liver microsomes and was well within the mCLint acceptance criteria (human: <8.60 low and >47.0 high, rat: 13.2 low and >71.9 high, mouse: <8.80 low and >48.0 high). The compound tested Compound 6 showed medium to highly stability across the species tested.

### Metabolic Stability Assay in Human Liver Microsomes in the Presence of Specific Cytochrome P450 Inhibitors

The experiment was performed in duplicate, and the final test concentration was 1 μM. The incubations were carried out for 45 min with intermediate time points of 0, 5, 15, and 30 min. CYP specific reference standard and inhibitors were used for this experiment. The tested concentration for substrates were 1 μM and 20 μM for inhibitors.

The following substrates and inhibitors were used:

| CYPs | Substrate | Inhibitor |
|---|---|---|
| 3A4 | Midazolam | Ketoconazole |
| 2D6 | Bufuralol | Quinidine |
| 2C9 | Diclofenac | Sulfaphenazole |
| 2C19 | Omeprazole | Nootkatone |
| 1A2 | Phenacetin | Furafylline |

For the microsomal stability experiment, vials containing the microsomes were thawed on the surface of an ice bath. 33 μL microsomes (20 mg/mL) was suspended in 1165.7 μL of 100 mM potassium phosphate buffer (pH 7.4) in the propylene tube labeled as incubation mixture. The control as well as test compounds will have similar set of incubation mixture tubes. 1.1 μL of compound of the disclosure such as Compound 6 (1 mM) was added to above incubation mixtures to give a working concentration of 1.1 μM respectively. Similarly, added 1.1 μL of Compound 6 (1 mM) and inhibitors (20 mM) to a second set of above incubation mixtures to give a working concentration of 1.1 μM and 20 μM respectively. 180 μL aliquot was transferred from respective incubation mixtures to 6 tubes labeled T_{Control}, T₀, T₅, T₁₅, T₃₀ and T₄₅.

All the tubes were pre-incubated at 37 t 1° C. for 5 min in a shaking water bath. Tubes of NADPH solution (10 mM) were similarly pre-incubated in similar conditions. After pre-incubation, 20 μL NADPH solution (10 mM) was added to the T₀, T₅, T₁₅, T₃₀, and T₄₅ tubes, and 20 μL of buffer was added to the T_{Control} tube which makes the final concentration to 1 μM. T₀ was immediately quenched with 200 μL of quenching solution containing warfarin as internal standard (IS). Similarly, at the end of the incubation period (5, 15, 30, and 45 min) of respective tubes, 200 μL of quenching solution containing warfarin as internal standard (IS) was added to each tube to stop the reaction. Resulting samples were centrifuged at 3220 g for 20 min. Supernatant (200 μL) from each reaction tube was taken for LC-MS/MS analysis. Calculations were done as noted in the earlier section.

The stability of Compound 6 when incubated human liver microsomes along with CYP specific substrates and inhibitors is tabulated below:

| | % rem @ 5 | % rem @ 15 | % rem @ 30 | % rem @ 45 | t_{1/2} | mCLint |
|---|---|---|---|---|---|---|
| Compound ID | min | min | min | min | (min) | (μL/min/mg) |
| Compound 6 | 93.5 | 92.9 | 91.3 | 67.8 | 96.3 | 14.4 |
| Midazolam 1 μM | 22.0 | 2.77 | <1 | <1 | 5.21 | 266 |
| Midazolam | 96.7 | 97.1 | ~100 | ~100 | NA | NA |
| 1 μM + | | | | | | |
| Ketoconazole | | | | | | |
| Compound 6 | 81.9 | 81.5 | 61.8 | 52.9 | 52.1 | 26.6 |
| 1 μM + | | | | | | |
| Ketoconazole | | | | | | |
| Burfuralol 1 μM | 88.0 | 76.3 | 67.7 | 55.4 | 56.6 | 24.5 |
| Burfuralol 1 μM + | 93.4 | 91.4 | 91.8 | 85.8 | 263 | 5.26 |
| Quinidine | | | | | | |
| Compound 6 | 80.8 | 67.3 | 57.4 | 60.1 | 64.2 | 21.6 |
| 1 μM + Quinidine | | | | | | |
| Diclofenac | 49.5 | 28.3 | 10.5 | 4.93 | 10.8 | 129 |
| 1 μM | | | | | | |
| Diclofenac | ~100 | ~100 | 91.9 | 82.0 | 131 | 10.6 |
| 1 μM + | | | | | | |
| Sulfaphenazole | | | | | | |
| Compound 6 | 69.7 | 63.3 | 73.8 | 54.3 | 77.1 | 18.0 |
| 1 μM + | | | | | | |
| Sulfaphenazole | | | | | | |
| Omeprazole 1 μM | 95.9 | 71.1 | 60.1 | 28.8 | 26.4 | 52.6 |
| Omeprazole | 94.8 | 92.7 | 85.0 | 55.6 | 59.3 | 23.4 |
| 1 μM + | | | | | | |
| Nootkatone | | | | | | |
| Compound 6 | ~100 | 98.6 | 84.6 | 58.7 | 54.6 | 25.4 |
| 1 μM + | | | | | | |
| Nootkatone | | | | | | |
| Phenacetin | 98.2 | 81.9 | 67.7 | 44.5 | 39.1 | 35.4 |
| 1 μM | | | | | | |
| Phenacetin | ~100 | ~100 | ~100 | 71.9 | 102 | 13.6 |
| 1 μM + | | | | | | |
| Furafylline | | | | | | |
| Compound 6 | 96.8 | 87.7 | 66.9 | 63.2 | 61.7 | 22.5 |
| 1 μM + | | | | | | |
| Furafylline | | | | | | |

Compound 6 compound showed medium to highly stability in human liver microsomes as in the previous section. The experiment was conducted in human liver microsomes and 5 different isoforms were targeted namely CYP 3A4, 2D6, 2C9, 2C19 and IA2. Compound 6 compound when incubated in human liver microsomes showed ˜35% degradation. The degradation of Compound 6 did not change much even when co-incubated with specific CYP inhibitors. The degradation of Compound 6 compound was between 40-45% even in presence of specific CYP inhibitors indicating that that more than one CYPs are involved in the degradation of Compound 6.

### Example 5—Analysis of Plasma Protein Binding. Permeability and Mode of Administration

### Plasma Protein Binding Assay

The movement of compounds of the disclosure such as Compound 6 from mouse, rat and human plasma towards the buffer through a membrane (12 kda cutoff) is tested by Equilibrium dialyzer method at a concentration of 3 μM for 4.5 h shaking at 37° C. Warfarin and naltrexone are to be used as positive controls and % bound, fraction unbound (fu) and percent recovery is calculated.

| | | (Apical | (Basal | ER (Basal | % recovery | % recovery |
|---|---|---|---|---|---|---|
| | | concentration- | concentration- | concentration-Apical | Apical | Basal |
| | | Basal | Apical | concentration/Apical | concentration- | concentration- |
| Compound | | concentration) | concentration) | concentration-Basal | Basal | Apical |
| Code | Assay Type | (10-6 cm/sec) | (10-6 cm/sec) | concentration) | concentration | concentration |
| Compound 6 | Permeability | 26.0 | 31.6 | 1.22 | 49.1 | ~100 |
| Digoxin | (MDR1-MDCK) | 0.28 | 15.2 | 54.3 | 99.9 | ~100 |
| Propranolol | @ 5 mM | 47.5 | 48.2 | 1.01 | 71.5 | ~100 |
| Lucifer yellow | | ND | ND | NA | 97.2 | 97.1 |

### Permeability Assay

The permeability of compounds of the disclosure such as Compound 6 from apical to basal direction and vice versa is determined through MDR1 transfected Madin-Darby canine kidney (MDCK) cell monolayer at a concentration of 5 μM for 60 min. Digoxin is used as reference and lucifer yellow is used as the integrity marker. The concentration of Compound 6 is determined by LC-MS/MS methods. Papp, efflux ratio and percent recovery are calculated. Results of Permeability assay is tabulated below.

Based on above results, compound 6 is a high permeable compound (A-B permeability is 26×10⁻⁶ cm/sec) and not a substrate of efflux (ER<2).

### Oral and Intravenous Pharmacokinetics in Rats

Plasma vs. time concentration profile along with key pharmacokinetic parameters such as AUC₀₋ₜ, AUC_{0-∞}, Cₘₐₓ, Tₘₐₓ, CL, Vd, t_{1/2} and F are determined post 10 mg/kg (oral) and 2 mg/kg (intravenous) administration in rats at 0.25, 0.5, 1, 2, 4, 6, 10 and 24 hrs for oral and 0.083, 0.25, 0.5, 1, 2, 4, 6, 10 and 24 h for intravenous treatment.

### Dose Escalation for Determining Maximum Tolerable Dosage (MTD) in Rats

MTD study is performed in 8-9 weeks old female Sprague Dawley rats with oral administration of four escalation doses. The rats are analyzed for body weight loss, appearance of any clinical signs, pathological symptoms or mortality. Plasma vs. time concentration profile along with key pharmacokinetic parameters as applicable (AUC₀₋ₜ, AUC_{0-∞}, Cₘₐₓ, Tₘₐₓ, CL, Vd, t_{1/2} and F) will be determined post 30, 100 and 300 mg/kg oral administration in rats at 0.25, 0.5, 1, 2, 4, 6, 10 and 24 hrs.

### Example 6—Toxicology Assay in Rats

The compounds of the disclosure such as Compound 6 are evaluated for a preliminary 4/14-day repeated dose escalating toxicity study in 6-8-week Sprague Dawley rats(male/female) at doses mentioned below. The general parameters such as Mortality, Bodyweight changes, Clinical signs, Urinalysis, Hematology, Blood biochemistry, Gross organ histopathology and No-observed-adverse-effect level (NOAEL dose) are monitored. The following doses are tested to determine the optimum dose for safety and efficacy.

| | | Route of | |
|---|---|---|---|
| Groups | Dose | administration | N |
| Sham control | — | — | 6 |
| Compound 6 | 25 mg/kg | Oral | 6 |
| Compound 6 | 50 mg/kg | Oral | 6 |
| Compound 6 | 100 mg/kg | Oral | 6 |
| Compound 6 | 300 mg/kg | Oral | 6 |
| Compound 6 | 800 mg/kg | Oral | 6 |

The following readouts are evaluated:

| Read outs | Parameter Estimate |
|---|---|
| Mortality | — |
| Body weight change | — |
| Clinical signs | Abnormal stools, food/water consumption, ECG, |
| | blood pressure, body temperature and |
| | ophthalmology |
| Haematology | Hb concentration, hematocrit, platelet/RBC/WBC |
| | count |
| Blood biochemistry | Glucose, triglycerides, creatinine, bilirubin, |
| | ALP, ALT |
| Urinalysis | Glucose, Protein, ketone bodies, crystals |
| Histopathology | Organ weight and histological changes in liver, |
| | kidney, heart, brain, GI tract, bone marrow |
| | and thymus |
| NOAEL dose | — |

The same experiment is then repeated for a 28-day GLP toxicity study following the same protocols as above.

### Example 7—Treatment of Primary Sclerosing Cholangitis

Primary sclerosing cholangitis (PSC) is a chronic liver disease in which the bile ducts inside and outside the liver become inflamed and scarred, and eventually narrowed or blocked. In primary sclerosing cholangitis, inflammation causes scars within the bile ducts. These scars make the ducts hard and narrow and gradually cause serious liver damage. A majority of people with primary sclerosing cholangitis also have inflammatory bowel disease, such as ulcerative colitis or Crohn's disease.

Mdr2 knockout mice is used as the animal model for Primary Sclerosing Cholangitis. 9-11 weeks old male FVB/NJ WT and Mdr2 knockout mice are randomized into different groups as indicated. The compounds of the disclosure such as Compound 6 are administered intraperitoneally or orally, daily, starting from 10 weeks of age to 12 weeks of age. At 12 weeks of age, the mice are sacrificed and liver and serum bile acid accumulation, liver fibrosis, pro-inflammatory and pro-fibrotic markers are analyzed. The following dose and routes of administration are tested:

| | | Route of | |
|---|---|---|---|
| Groups | Dose | administration | N |
| Sham control | — | — | 6 |
| Pathological control | — | — | 6 |
| Compound 6 | 1 mg/kg | Intraperitoneal | 6 |
| Compound 6 | 10 mg/kg | Intraperitoneal | 6 |
| Compound 6 | 30 mg/kg | Intraperitoneal | 6 |
| Compound 6 | 10 mg/kg | Oral | 6 |
| Compound 6 | 30 mg/kg | Oral | 6 |

### Example 8—Treatment of Arthritis

Arthritis is the swelling and tenderness of one or more joints. The main symptoms of arthritis are joint pain and stiffness, which typically worsen with age. The most common types of arthritis are osteoarthritis and rheumatoid arthritis. Osteoarthritis causes cartilage that covers the ends of bones where they form a joint to break down. Rheumatoid arthritis is a disease in which the immune system attacks the joints, beginning with the lining of joints. Monoclonal antibody induced arthritis model (mAb-induced RA, AIA or CAIA) is ideal for rapidly screening and evaluating anti-inflammatory therapeutic agents.

The compounds of the disclosure such as Compound 6 are evaluated for their ability to treat arthritis using Monoclonal antibody induced arthritis model. 8-10 weeks old male Balb/c are used in the assay. The mice are intraperitoneally (I.P) injected with cocktail of 5 monoclonal antibodies anti-type II collagen (1.5 mg). IP injection of 50 μg of lipopolysaccharide (LPS from Escherichia coli strain 055B5; in a sterile normal saline) are given on day 3.

| | | Route of | |
|---|---|---|---|
| Groups | Dose | administration | N |
| Sham control | — | — | 6 |
| Pathological control | — | — | 6 |
| Compound 6 | 1 mg/kg | Intraperitoneal | 6 |
| Compound 6 | 10 mg/kg | Intraperitoneal | 6 |
| Compound 6 | 30 mg/kg | Intraperitoneal | 6 |
| Compound 6 | 10 mg/kg | Oral | 6 |
| Compound 6 | 30 mg/kg | Oral | 6 |

Compound 6 is administered from day 2 to 10 at the doses mentioned below. The compounds will be administered both via intraperitoneal and oral route. Paw thickness, paw weight, clinical score, joint cytokine profile and histopathology are evaluated to determine if there is an improvement in these parameters post administration of compound.

### Additional Embodiments

1. A compound of Formula (I)

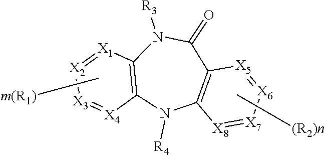

wherein R1 and R2 are each independently selected from the group consisting of hydrogen, —CO-alkyl, hydroxyl, halo, halo alkyl (C1-C6), trihalo alkyl (C1-C6), halo alkoxy, amino, C1-C6-alkyl-amino; wherein m and n are integers having each independently a value of 0, 1, 2, 3 or 4; wherein X1, X2, X3, X4, X5, X6, X7 and X8 is each independently selected from the group consisting of —CH and N;
wherein R3 is each independently selected from the group consisting of hydrogen, C1-C6 alkyl, trihalo alkyl (C1-C6), —CO-alkyl, and —CO-halo alkyl;
wherein R4 is each independently one of hydrogen or COY, with the proviso that R4 is not hydrogen when R3 is hydrogen and when X1-X8 is —CH, except that R4 and R3 may both be hydrogen when X1-X8 is N and/or R1 or R2 are a halo; or

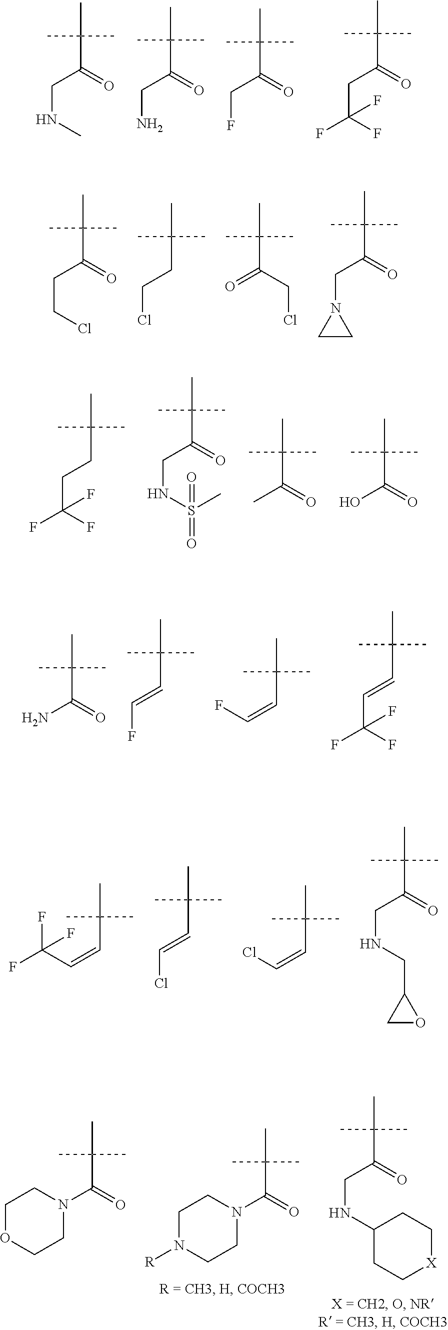

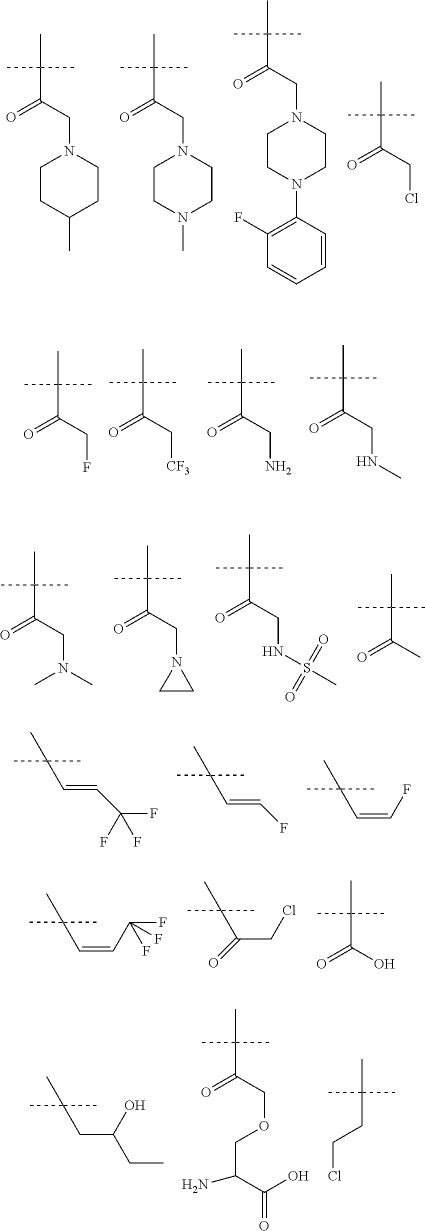

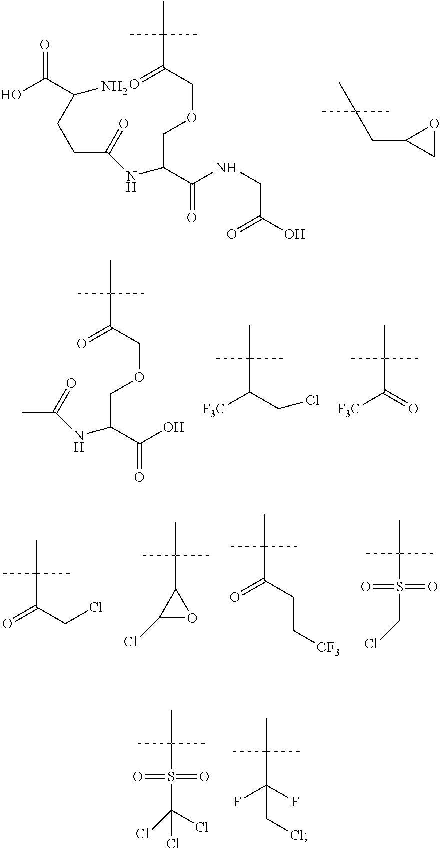

wherein Y is each independently selected from the group consisting of hydrogen, C1-C6 halo alkyl, C1-C6 halo alkoxy, C1-C6 amino alkyl, C1-C6 amino alkoxy, C3-C8 cyclo-(halo)-alkyl and wherein the alkyl or cycloalkyl group is optionally substituted with a five- or six-membered ring optionally containing at least one heteroatom selected from N, S and O, and wherein the five- or six-membered ring is optionally mono- or poly-substituted with C1-C6 alkyl, halo, C1-C6 halo alkyl, C1-C6 halo alkoxy, C1-C6 amino alkyl, C1-C6 amino alkoxy, C3-C8 cycloalkyl, C3-C8 cycloalkyl substituted with halo, amino, carboxyl or alkoxy group.
2. The compound of innovation 1, wherein R4 is not hydrogen, m is zero and n is 1.
3. The compound of innovation 2, wherein R4 is COY and Y is a substituted piperazine.
4. The compound of innovation 2, wherein R4 is COY and Y is a halo alkyl.
5. The compound of innovation 1, wherein R4 is hydrogen, m is zero, n is one and R2 is halo
6. The compound of innovation 2, wherein R4 is COY and Y is a substituted piperidine.
7. A compound selected from the group consisting of:

| Name | Compound Structure | IUPAC Name |
|---|---|---|
| Compound 1 | 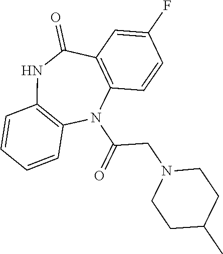 [SMILES: CC1CCN(CC(=O)N2c3ccccc3NC(=O)c3cc(F)ccc32)CC1] | 2-fluoro-5-(2-(4- methylpiperidin-1-yl)acetyl)- 5,10-dihydro-11H- dibenzo[b,e][1,4]diazepin-11- one |
| Compound 2 | 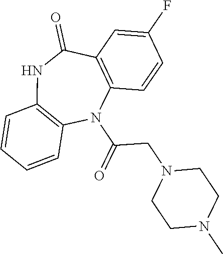 [SMILES: CN1CCN(CC(=O)N2c3ccccc3NC(=O)c3cc(F)ccc32)CC1] | 2-fluoro-5-(2-(4- methylpiperidin-1-yl)acetyl)- 5,10-dihydro-11H- dibenzo[b,e][1,4]diazepin-11- one |
| Compound 3 | 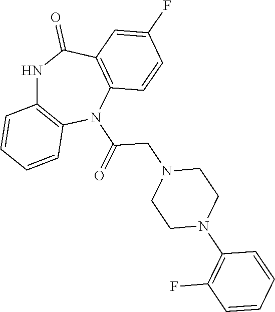 [SMILES: O=C1Nc2ccccc2N(C(=O)CN2CCN(c3ccccc3F)CC2)c2ccc(F)cc21] | 2-fluoro-5-(2-(4-(2- fluorophenyl)piperazin-1- yl)acetyl)-5,10-dihydro-11H- dibenzo[b,e][1,4]diazepin-11- one |
| Compound 4 | 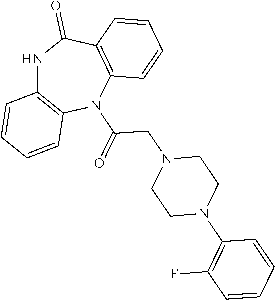 [SMILES: O=C1Nc2ccccc2N(C(=O)CN2CCN(c3ccccc3F)CC2)c2ccccc21] | 5-(2-chloroacetyl)-5,10-dihydro- 11H-dibenzo[b,e][1,4]diazepin- 11-one |
| Compound 5 | 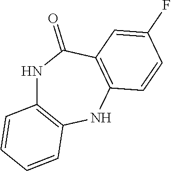 [SMILES: O=C1Nc2ccccc2Nc2ccc(F)cc21] | 2-fluoro-5,10-dihydro-11H- dibenzo[b,e][1,4]diazepin-11- one |
| Compound 6 | 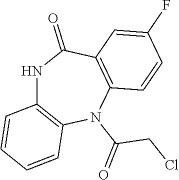 [SMILES: O=C1Nc2ccccc2N(C(=O)CCl)c2ccc(F)cc21] | 5-(2-chloroacetyl)-2-fluoro- 5,10-dihydro-11 H- dibenzo[b,e][1,4]diazepin-11- one |
| Compound 7 | 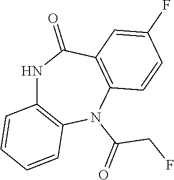 [SMILES: O=C1Nc2ccccc2N(C(=O)CF)c2ccc(F)cc21] | 2-fluoro-5-(fluoroacetyl)-5,10- dihydro-11H- dibenzo[b,e][1,4]diazepin-11- one |
| Compound 8 | 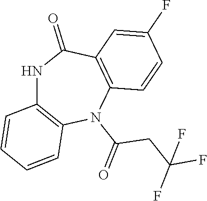 [SMILES: O=C1Nc2ccccc2N(C(=O)CC(F)(F)F)c2ccc(F)cc21] | 2-fluoro-5-(3,3,3- trifluoropropanoyl)-5,10- dihydro-11H- dibenzo[b,e][1,4]diazepin-11- one |
| Compound 9 | 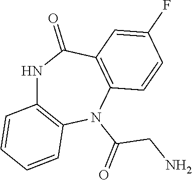 [SMILES: NCC(=O)N1c2ccccc2NC(=O)c2cc(F)ccc21] | 5-(aminoacetyl)-2-fluoro-5,10- dihydro-11H- dibenzo[b,e][1,4]diazepin-11- one |
| Compound 10 | 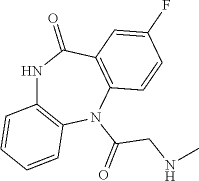 [SMILES: CNCC(=O)N1c2ccccc2NC(=O)c2cc(F)ccc21] | 2-fluoro-5- [(methylamino)acetyl]-5,10- dihydro-11H- dibenzo[b,e][1,4]diazepin-11- one |
| Compound 11 | 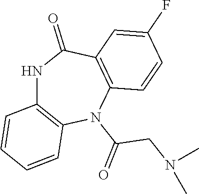 [SMILES: CN(C)CC(=O)N1c2ccccc2NC(=O)c2cc(F)ccc21] | 5-[(dimethylamino)acetyl]-2- fluoro-5,10-dihydro-11H- dibenzo[b,e][1,4]diazepin-11- one |
| Compound 12 | 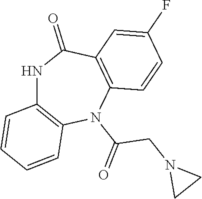 [SMILES: O=C1Nc2ccccc2N(C(=O)CN2CC2)c2ccc(F)cc21] | 5-[(aziridin-1-yl)acetyl]-2- fluoro-5,10-dihydro-11H- dibenzo[b,e][1,4]diazepin-11- one |
| Compound 13 | 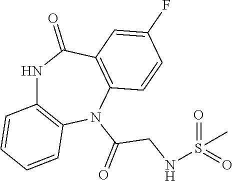 [SMILES: CS(=O)(=O)NCC(=O)N1c2ccccc2NC(=O)c2cc(F)ccc21] | N-[2-(2-fluoro-11-oxo-10,11- dihydro-5H- dibenzo[b,e][1,4]diazepin-5-yl)- 2-oxoethyl]methanesulfonamide |
| Compound 14 | 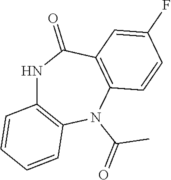 [SMILES: CC(=O)N1c2ccccc2NC(=O)c2cc(F)ccc21] | 5-acetyl-2-fluoro-5,10-dihydro- 11H-dibenzo[b,e][1,4]diazepin- 11-one |
| Compound 15 | 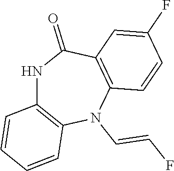 [SMILES: O=C1Nc2ccccc2N(/C=C/F)c2ccc(F)cc21] | 2-fluoro-5-[(E)-2- fluoroethenyl]-5,10-dihydro- 11H-dibenzo[b,e][1,4]diazepin- 11-one |
| Compound 16 | 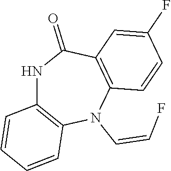 [SMILES: O=C1Nc2ccccc2N(/C=C\F)c2ccc(F)cc21] | 2-fluoro-5-[(Z)-2- fluoroethenyl]-5,10-dihydro- 11H-dibenzo[b,e][1,4]diazepin- 11-one |
| Compound 17 | 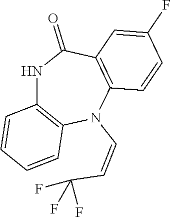 [SMILES: O=C1Nc2ccccc2N(/C=C\C(F)(F)F)c2ccc(F)cc21] | 2-fluoro-5-[(1Z)-3,3,3- trifluoroprop-1-en-1-yl]-5,10- dihydro-11H- dibenzo[b,e][1,4]diazepin-11- one |
| Compound 18 | 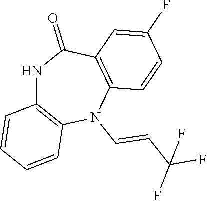 [SMILES: O=C1Nc2ccccc2N(/C=C/C(F)(F)F)c2ccc(F)cc21] | 2-fluoro-5-[(1E)-3,3,3- trifluoroprop-1-en-1-yl]-5,10- dihydro-11H- dibenzo[b,e][1,4]diazepin-11- one |
| Compound 19 | 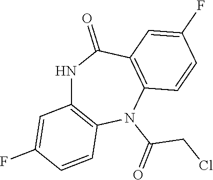 [SMILES: O=C1Nc2cc(F)ccc2N(C(=O)CCl)c2ccc(F)cc21] | 5-(chloroacetyl)-2-fluoro-10- methyl-5,10-dihydro-11H- dibenzo[b,e][1,4]diazepin-11- one |
| Compound 20 | 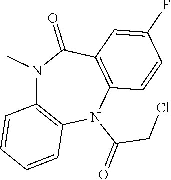 [SMILES: CN1C(=O)c2cc(F)ccc2N(C(=O)CCl)c2ccccc21] | 5-(chloroacetyl)-2-fluoro-10- methyl-5,10-dihydro-11H- dibenzo[b,e][1,4]diazepin-11- one |
| Compound 21 | 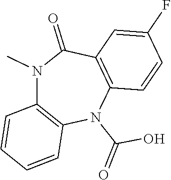 [SMILES: CN1C(=O)c2cc(F)ccc2N(C(=O)O)c2ccccc21] | 2-fluoro-10-methyl-11-oxo- 10,11-dihydro-5H- dibenzo[b,e][1,4]diazepine-5- carboxylic acid |
| Compound 22 | 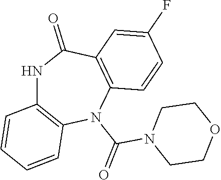 [SMILES: O=C1Nc2ccccc2N(C(=O)N2CCOCC2)c2ccc(F)cc21] | 2-fluoro-5-(morpholine-4- carbonyl)-5,10-dihydro-11H- dibenzo[b,e][1,4]diazepin-11- one |
| Compound 23 | 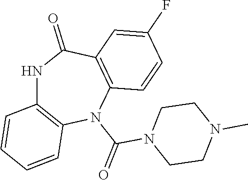 [SMILES: CN1CCN(C(=O)N2c3ccccc3NC(=O)c3cc(F)ccc32)CC1] | 2-fluoro-5-(4-methylpiperazine- 1-carbonyl)-5,10-dihydro-11H- dibenzo[b,e][1,4]diazepin-11- one |
| Compound 24 | 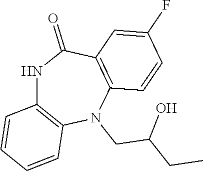 [SMILES: CCC(O)CN1c2ccccc2NC(=O)c2cc(F)ccc21] | 2-fluoro-5-(2-hydroxybutyl)- 5,10-dihydro-11H- dibenzo[b,e][1,4]diazepin-11- one |
| Compound 25 | 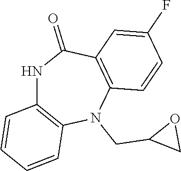 [SMILES: O=C1Nc2ccccc2N(CC2CO2)c2ccc(F)cc21] | 2-fluoro-5-[(oxiran-2- yl)methyl]-5,10-dihydro-11H- dibenzo[b,e][1,4]diazepin-11- one |
| Compound 26 | 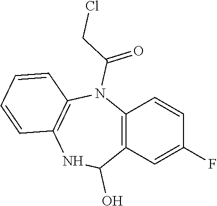 [SMILES: O=C(CCl)N1c2ccccc2NC(O)c2cc(F)ccc21] | 2-chloro-1-(2-fluoro-11- hydroxy-10,11-dihydro-5H- dibenzo[b,e][1,4]diazepin-5- yl)ethan-1-one |
| Compound 27 | 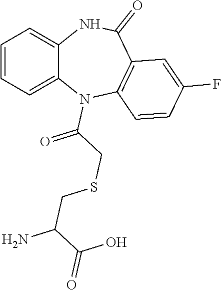 [SMILES: NC(CSCC(=O)N1c2ccccc2NC(=O)c2cc(F)ccc21)C(=O)O] | 2-amino-3-{[2-(2-fluoro-11- oxo-10,11-dihydro-5H- dibenzo[b,e][1,4]diazepin-5-yl)- 2-oxoethyl] sulfanyl} propanoic acid |
| Compound 28 | 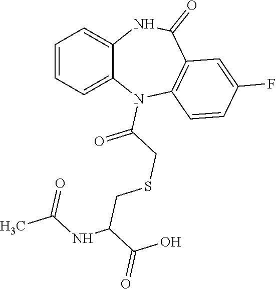 [SMILES: CC(=O)NC(CSCC(=O)N1c2ccccc2NC(=O)c2cc(F)ccc21)C(=O)O] | 2-acetamido-3-{[2-(2-fluoro-11- oxo-10,11-dihydro-5H- dibenzo[b,e][1,4]diazepin-5-yl)- 2-oxoethyl]sulfanyl}propanoic acid |
| Compound 29 | 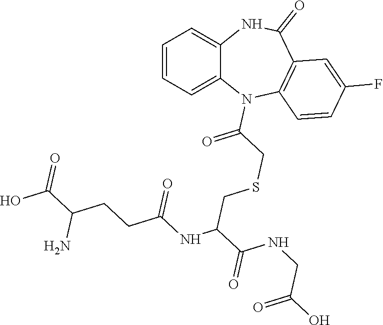 [SMILES: NC(CCC(=O)NC(CSCC(=O)N1c2ccccc2NC(=O)c2cc(F)ccc21)C(=O)NCC(=O)O)C(=O)O] | 2-amino-5-((1- ((carboxymethyl)amino)-3-((2- (2-fluoro-11-oxo-10,11-dihydro- 5H-dibenzo[b,e][1,4]diazepin-5- yl)-2-oxoethyl)thio)-1- oxopropan-2-yl)amino)-5- oxopentanoic acid |
| Compound 30 | 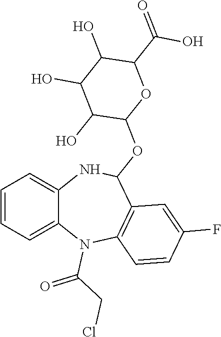 [SMILES: O=C(O)C1OC(OC2Nc3ccccc3N(C(=O)CCl)c3ccc(F)cc32)C(O)C(O)C1O] | 6-((5-(2-chloroacetyl)-2-fluoro- 10,11-dihydro-5H- dibenzo[b,e][1,4]diazepin-11- yl)oxy)-3,4,5- trihydroxytetrahydro-2H-pyran- 2-carboxylic acid |
| Compound 31 | 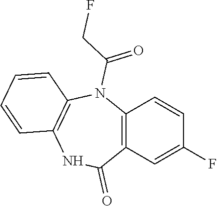 [SMILES: O=C1Nc2ccccc2N(C(=O)CF)c2ccc(F)cc21] | 2-fluoro-5-(fluoroacetyl)-5,10- dihydro-11H- dibenzo[b,e][1,4]diazepin-11- one |
| Compound 32 | 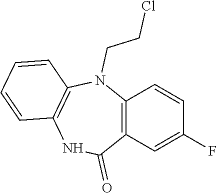 [SMILES: O=C1Nc2ccccc2N(CCCl)c2ccc(F)cc21] | 5-(2-chloroethyl)-2-fluoro-5,10- dihydro-11H- dibenzo[b,e][1,4]diazepin-11- one |
| Compound 33 | 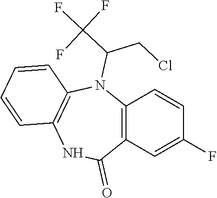 [SMILES: O=C1Nc2ccccc2N(C(CCl)C(F)(F)F)c2ccc(F)cc21] | 5-(3-chloro-1,1,1- trifluoropropan-2-yl)-2-fluoro- 5,10-dihydro-11H- dibenzo[b,e][1,4]diazepin-11- one |
| Compound 34 | 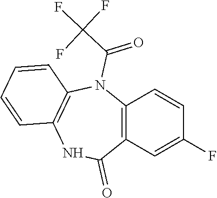 [SMILES: O=C1Nc2ccccc2N(C(=O)C(F)(F)F)c2ccc(F)cc21] | 2-fluoro-5-(trifluoroacetyl)- 5,10-dihydro-11H- dibenzo[b,e][1,4]diazepin-11- one |
| Compound 35 | 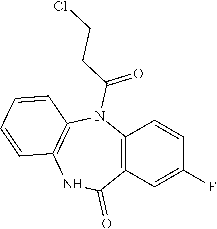 [SMILES: O=C1Nc2ccccc2N(C(=O)CCCl)c2ccc(F)cc21] | 5-(3-chloropropanoyl)-2-fluoro- 5,10-dihydro-11H- dibenzo[b,e][1,4]diazepin-11- one |
| Compound 36 | 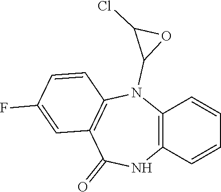 [SMILES: O=C1Nc2ccccc2N(C2OC2Cl)c2ccc(F)cc21] | 5-(3-chlorooxiran-2-yl)-2- fluoro-5,10-dihydro-11H- dibenzo[b,e][1,4]diazepin-11- one |
| Compound 37 | 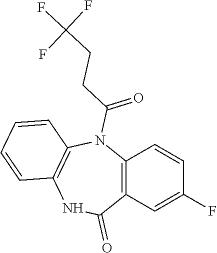 [SMILES: O=C1Nc2ccccc2N(C(=O)CCC(F)(F)F)c2ccc(F)cc21] | 2-fluoro-5-(4,4,4- trifluorobutanoyl)-5,10-dihydro- 11H-dibenzo[b,e][1,4]diazepin- 11-one |
| Compound 38 | 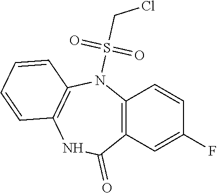 [SMILES: O=C1Nc2ccccc2N(S(=O)(=O)CCl)c2ccc(F)cc21] | 5-(chloromethanesulfonyl)-2- fluoro-5,10-dihydro-11H- dibenzo[b,e][1,4]diazepin-11- one |
| Compound 39 | 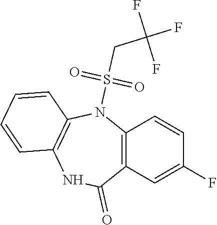 [SMILES: O=C1Nc2ccccc2N(S(=O)(=O)CC(F)(F)F)c2ccc(F)cc21] | 2-fluoro-5-(2,2,2- trifluoroethanesulfonyl)-5,10- dihydro-11H- dibenzo[b,e][1,4]diazepin-11- one |
| Compound 40 | 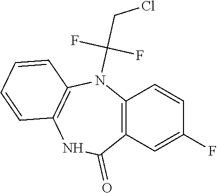 [SMILES: O=C1Nc2ccccc2N(C(F)(F)CCl)c2ccc(F)cc21] | 5-(2-chloro-1,1-difluoroethyl)-2- fluoro-5,10-dihydro-11H- dibenzo[b,e][1,4]diazepin-11- one |
| Compound 41 | 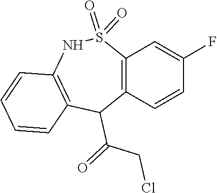 [SMILES: O=C(CCl)C1c2ccccc2NS(=O)(=O)c2cc(F)ccc21] | 11-(chloroacetyl)-3-fluoro-6,11- dihydro-5H-526- dibenzo[c,f][1,2]thiazepine-5,5- dione |
| Compound 42 | 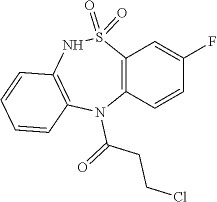 [SMILES: O=C(CCCl)N1c2ccccc2NS(=O)(=O)c2cc(F)ccc21] | 11-(3-chloropropanoyl)-3- fluoro-6,11-dihydro-5H-5λ6- dibenzo[c,f][1,2,5]thiadiazepine- 5,5-dione |
| Compound 43 | 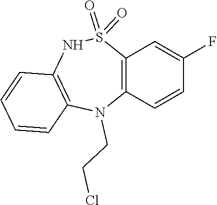 [SMILES: O=S1(=O)Nc2ccccc2N(CCCl)c2ccc(F)cc21] | 11-(2-chloroethyl)-3-fluoro- 6,11-dihydro-5H-5λ6- dibenzo[c,f][1,2,5]thiadiazepine- 5,5-dione |
| Compound 44 | 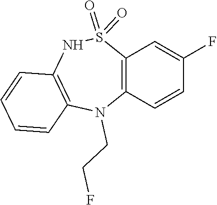 [SMILES: O=S1(=O)Nc2ccccc2N(CCF)c2ccc(F)cc21] | 3-fluoro-11-(2-fluoroethyl)- 6,11-dihydro-5H-5λ6- dibenzo[c,f][1,2,5]thiadiazepine- 5,5-dione |
| Compound 45 | 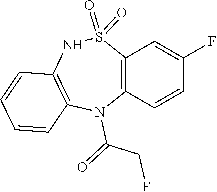 [SMILES: O=C(CF)N1c2ccccc2NS(=O)(=O)c2cc(F)ccc21] | 3-fluoro-11-(fluoroacetyl)-6,11- dihydro-5H-5λ6- dibenzo[c,f][1,2,5]thiadiazepine- 5,5-dione |
| Compound 46 | 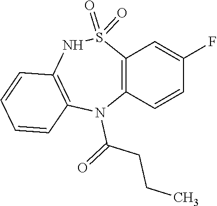 [SMILES: CCCC(=O)N1c2ccccc2NS(=O)(=O)c2cc(F)ccc21] | 11-butanoyl-3-fluoro-6,11- dihydro-5H-5λ6- dibenzo[c,f][1,2,5]thiadiazepine- 5,5-dione |
| Compound 47 | 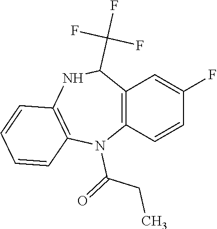 [SMILES: CCC(=O)N1c2ccccc2NC(C(F)(F)F)c2cc(F)ccc21] | 1-[2-fluoro-11- (trifluoromethyl)-10,11-dihydro- 5H-dibenzo[b,e][1,4]diazepin-5- yl]propan-1-one |
| Compound 48 | 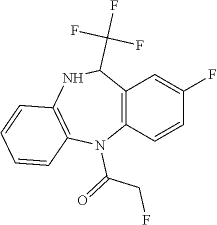 [SMILES: O=C(CF)N1c2ccccc2NC(C(F)(F)F)c2cc(F)ccc21] | 2-fluoro-1-[2-fluoro-11- (trifluoromethyl)-10,11-dihydro- 5H-dibenzo[b,e][1,4]diazepin-5- yl]ethan-1-one |
| Compound 49 | 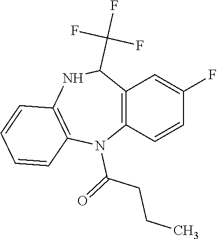 [SMILES: CCCC(=O)N1c2ccccc2NC(C(F)(F)F)c2cc(F)ccc21] | 1-[2-fluoro-11- (trifluoromethyl)-10,11-dihydro- 5H-dibenzo[b,e][1,4]diazepin-5- yl]butan-1-one |
| Compound 50 | 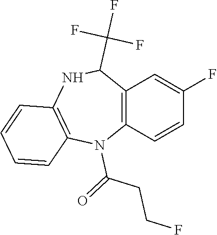 [SMILES: O=C(CCF)N1c2ccccc2NC(C(F)(F)F)c2cc(F)ccc21] | 3-fluoro-1-[2-fluoro-11- (trifluoromethyl)-10,11-dihydro- 5H-dibenzo[b,e][1,4]diazepin-5- yl]propan-1-one |
| Compound 51 | 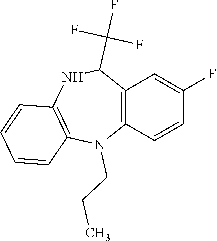 [SMILES: CCCN1c2ccccc2NC(C(F)(F)F)c2cc(F)ccc21] | 2-fluoro-5-propyl-11- (trifluoromethyl)-10,11-dihydro- 5H-dibenzo[b,e][1,4]diazepine |
| Compound 52 | 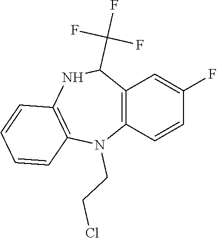 [SMILES: Fc1ccc2c(c1)C(C(F)(F)F)Nc1ccccc1N2CCCl] | 5-(2-chloroethyl)-2-fluoro-11- (trifluoromethyl)-10,11-dihydro- 5H-dibenzo[b,e][1,4]diazepine |
| Compound 53 | 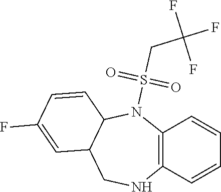 [SMILES: O=S(=O)(CC(F)(F)F)N1c2ccccc2NCC2C=C(F)C=CC21] | 2-fluoro-5-(2,2,2- trifluoroethanesulfonyl)- 5,10,11,11a-tetrahydro-4aH- dibenzo[b,e][1,4]diazepine |
| Compound 54 | 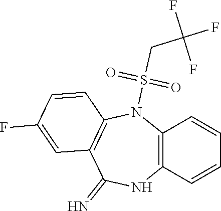 [SMILES: N=C1Nc2ccccc2N(S(=O)(=O)CC(F)(F)F)c2ccc(F)cc21] | 2-fluoro-5-(2,2,2- trifluoroethanesulfonyl)-5,10- dihydro-11H- dibenzo[b,e][1,4]diazepin-11- imine |
| Compound 55 | 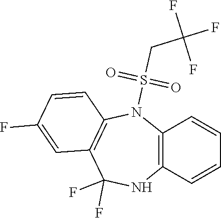 [SMILES: O=S(=O)(CC(F)(F)F)N1c2ccccc2NC(F)(F)c2cc(F)ccc21] | 2,11,11-trifluoro-5-(2,2,2- trifluoroethanesulfonyl)-10,11- dihydro-5H- dibenzo[b,e][1,4]diazepine |
| Compound 56 | 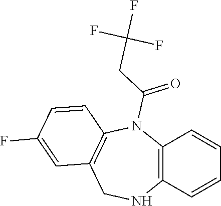 [SMILES: O=C(CC(F)(F)F)N1c2ccc(F)cc2CNc2ccccc21] | 3,3,3-trifluoro-1-(2-fluoro- 10,11-dihydro-5H- dibenzo[b,e][1,4]diazepin-5- yl)propan-1-one |
| Compound 57 | 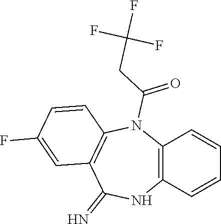 [SMILES: N=C1Nc2ccccc2N(C(=O)CC(F)(F)F)c2ccc(F)cc21] | 3,3,3-trifluoro-1-(2-fluoro-11- imino-10,11-dihydro-5H- dibenzo[b,e][1,4]diazepin-5- yl)propan-1-one |
| Compound 58 | 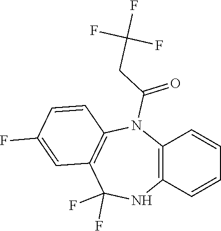 [SMILES: O=C(CC(F)(F)F)N1c2ccccc2NC(F)(F)c2cc(F)ccc21] | 3,3,3-trifluoro-1-(2,11,11- trifluoro-10,11-dihydro-5H- dibenzo[b,e][1,4]diazepin-5- yl)propan-1-one |
| Compound 59 | 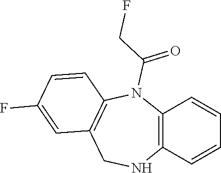 [SMILES: O=C(CF)N1c2ccc(F)cc2CNc2ccccc21] | 2-fluoro-1-(2-fluoro-10,11- dihydro-5H- dibenzo[b,e][1,4]diazepin-5- yl)ethan-1-one |
| Compound 60 | 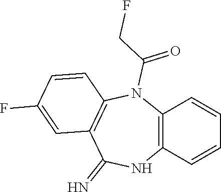 [SMILES: N=C1Nc2ccccc2N(C(=O)CF)c2ccc(F)cc21] | 2-fluoro-1-(2-fluoro-11-imino-5 10,11-dihydro-5H- dibenzo[b,e][1,4]diazepin-5- yl)ethan-1-one |
| Compound 61 | 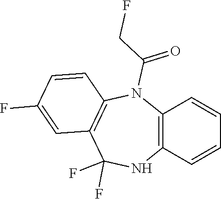 [SMILES: O=C(CF)N1c2ccccc2NC(F)(F)c2cc(F)ccc21] | 2-fluoro-1-(2,11,11-trifluoro- 10,11-dihydro-5H- dibenzo[b,e][1,4]diazepin-5- yl)ethan-1-one |
| Compound 62 | 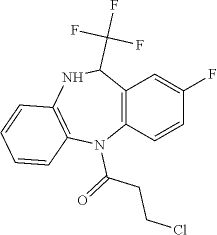 [SMILES: O=C(CCCl)N1c2ccccc2NC(C(F)(F)F)c2cc(F)ccc21] | 3-chloro-1-[2-fluoro-11- (trifluoromethyl)-10,11-dihydro- 5H-dibenzo[b,e][1,4]diazepin-5- yl]propan-1-one |

8. The compound of any one of innovations 1-7, wherein said compound has an IC50 value of about 2 μm.
9. The compound of any one of innovations 1-8, wherein said compound is capable of reducing the expression of IL-1β by at least 50%.
10. The compound of any one of innovations 1-9, wherein said compound is capable of treating inflammatory diseases.
11. A method of treating diseases of inflammation comprising the step of administering the compound of any one of innovations 1-10 and thereby treating said disease.
12. The method of innovation 11, wherein said disease is selected from the group consisting of inflammatory bowel disease (IBD), irritable bowel syndrome (IBS), Primary Sclerosing Cholangitis, primary biliary cirrhosis, alcoholic hepatitis, alcoholic liver cirrhosis, pancreatitis, non-alcoholic steatohepatitis, alcoholic pancreatitis, acute hepatitis, celiac disease, Non-steroidal anti-inflammatory drug (NSAID)-induced ulcer, gastric ulcer, antiphospholipid syndrome, Barrett's esophagus, postoperative ileus, atrophic gastritis, peritonitis, diverticulitis, duodenal ulcer, alveolar periostitis, Crohn's disease, Alzheimer's disease, arthritis, and multiple sclerosis.
13. A compound of formula I(a)

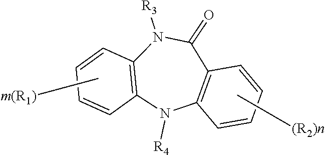

wherein R1 and R2 are each independently selected from the group consisting of hydrogen, —CO-alkyl, hydroxyl, halo, halo alkyl (C1-C6), trihalo alkyl (C1-C6), halo alkoxy, amino, C1-C6-alkyl-amino;
wherein m and n are integers having each independently a value of 0, 1, 2, 3 or 4;
wherein R3 is each independently selected from the group consisting of hydrogen, C1-C6 alkyl, trilhalo alkyl (C1-C6), and —CO-alkyl;
wherein R4 is each independently one of hydrogen or COY with the proviso that R4 is not hydrogen when R3 is hydrogen, except that R4 and R3 may both be hydrogen when either R1 or R2 is halo, or

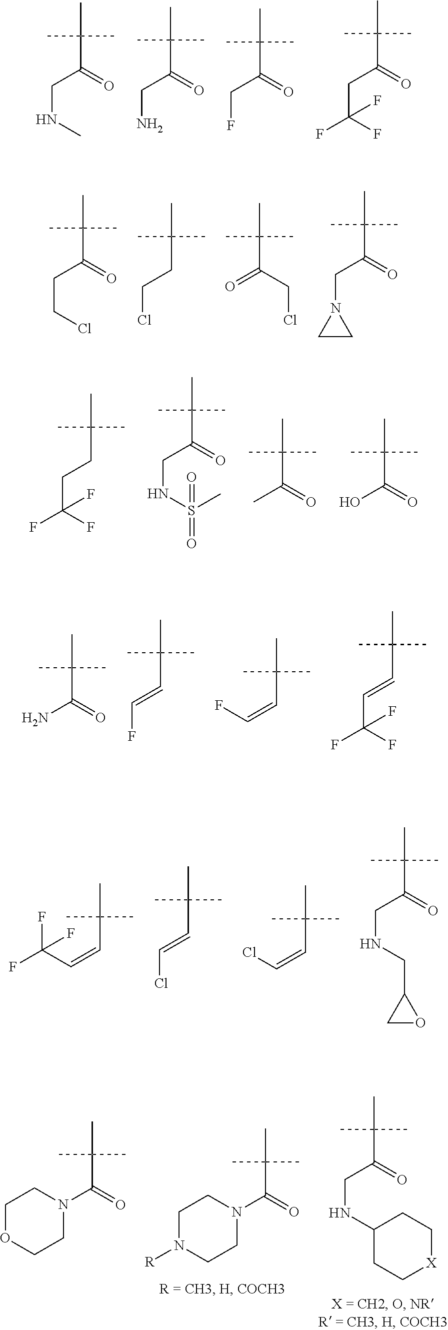

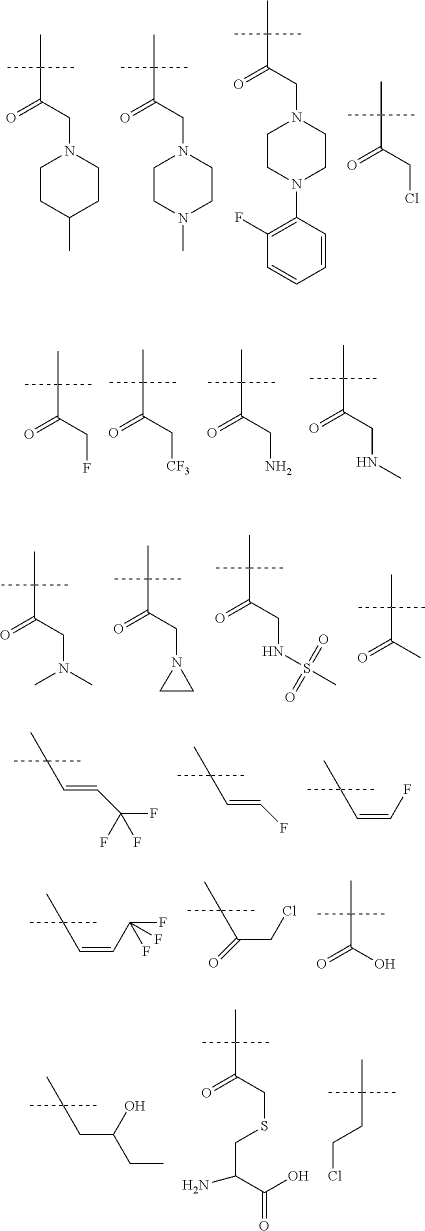

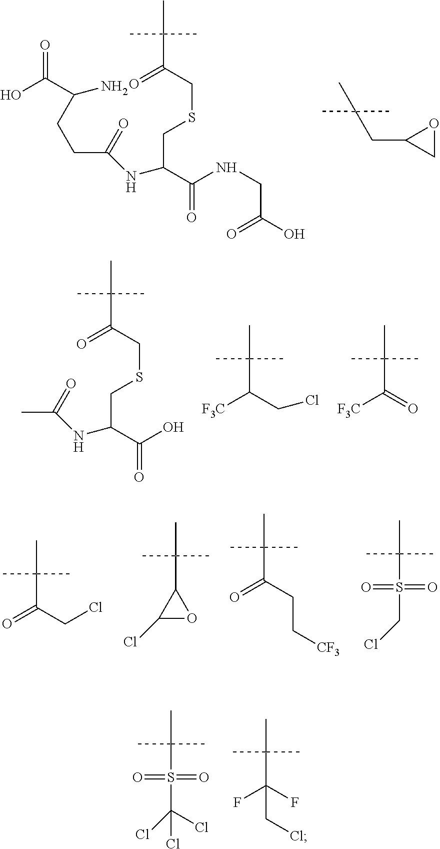

wherein Y is each independently selected from the group consisting of hydrogen, C1-C6 halo alkyl, C1-C6 halo alkoxy, C1-C6 amino alkyl, C1-C6 amino alkoxy, C3-C8 cyclo-(halo)-alkyl and wherein the alkyl or cycloalkyl group is optionally substituted with a five- or six-membered ring optionally containing at least one heteroatom selected from N, S and O, and wherein the five- or six-membered ring is optionally mono- or poly-substituted with C1-C6 alkyl, halo, C1-C6 halo alkyl, C1-C6 halo alkoxy, C1-C6 amino alkyl, C1-C6 amino alkoxy, C3-C8 cycloalkyl, C3-C8 cycloalkyl substituted with halo, amino, carboxyl or alkoxy group.
14. A compound of formula I(b)

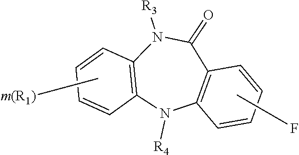

wherein R1 is each independently selected from the group consisting of hydrogen, —CO— alkyl, hydroxyl, halo, halo alkyl (C1-C6), trihalo alkyl (C1-C6), halo alkoxy, amino, C1-C6-alkyl-amino; wherein m is an integer having each independently a value of 0, 1, 2, 3 or 4;
wherein R3 is each independently selected from the group consisting of hydrogen, C1-C6 alkyl, trilhalo alkyl (C1-C6), and —CO-alkyl;
wherein R4 is each independently one of hydrogen or COY or

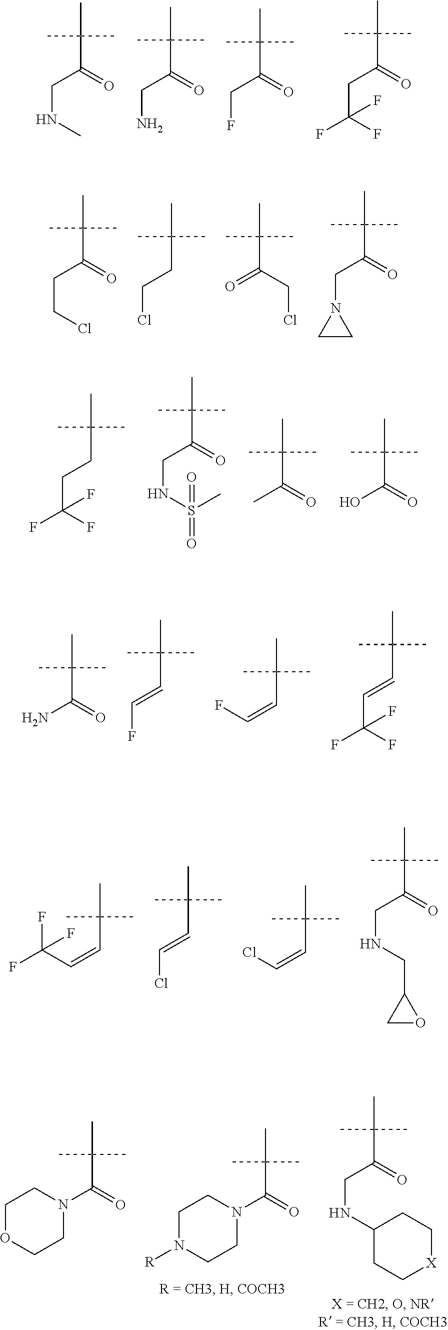

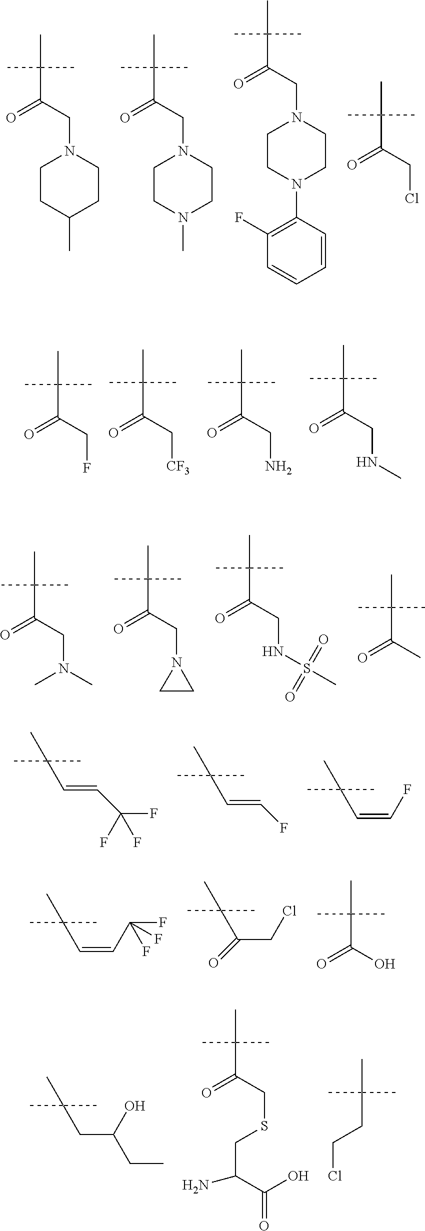

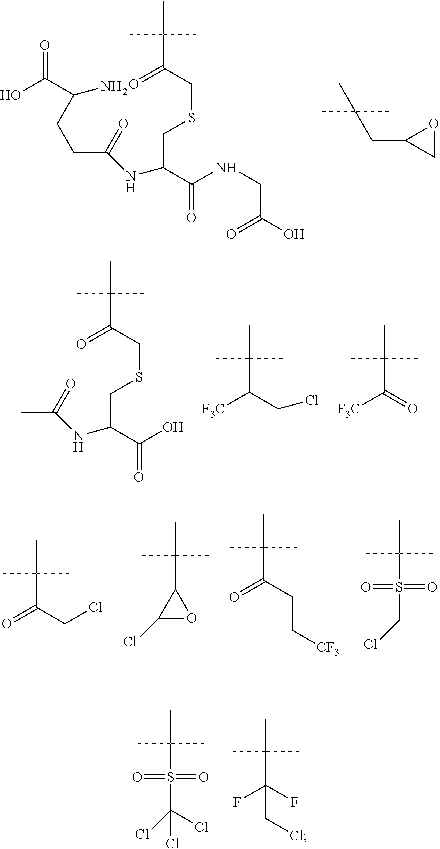

wherein Y is each independently selected from the group consisting of hydrogen, C1-C6 halo alkyl, C1-C6 halo alkoxy, C1-C6 amino alkyl, C1-C6 amino alkoxy, C3-C8 cyclo-(halo)-alkyl and wherein the alkyl or cycloalkyl group is optionally substituted with a five- or six-membered ring optionally containing at least one heteroatom selected from N, S and O, and wherein the five- or six-membered ring is optionally mono- or poly-substituted with C1-C6 alkyl, halo, C1-C6 halo alkyl, C1-C6 halo alkoxy, C1-C6 amino alkyl, C1-C6 amino alkoxy, C3-C8 cycloalkyl, C3-C8 cycloalkyl substituted with halo, amino, carboxyl or alkoxy group.
15. The compound of any one of innovations 13 or 14, wherein said compound is capable of reducing the expression of IL-1β by at least 50%.

### OTHER EMBODIMENTS

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

The following are exemplary claims directed to the subject matter described above and should not be considered to limit the present invention; Applicant reserves the right to pursue claims to any of the disclosed subject matter:

1. A compound of Formula (I) 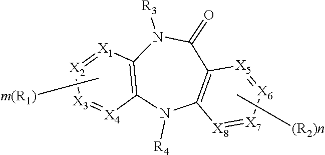
wherein R1 and R2 are each independently selected from the group consisting of hydrogen, —CO-alkyl, hydroxyl, halo, halo alkyl (C1-C6), trihalo alkyl (C1-C6), halo alkoxy, amino, C1-C6-alkyl-amino; wherein m and n are integers having each independently a value of 0, 1, 2, 3 or 4;
wherein X1, X2, X3, X4, X5, X6, X7 and X8 is each independently selected from the group consisting of —CH and N;
wherein R3 is each independently selected from the group consisting of hydrogen, C1-C6 alkyl, trihalo alkyl (C1-C6), —CO-alkyl, and —CO-halo alkyl;
wherein R4 is each independently one of hydrogen or COY, with the proviso that R4 is not hydrogen when R3 is hydrogen and when X1-X8 is —CH, except that R4 and R3 may both be hydrogen when X1-X8 is N and/or R1 or R2 are a halo; or 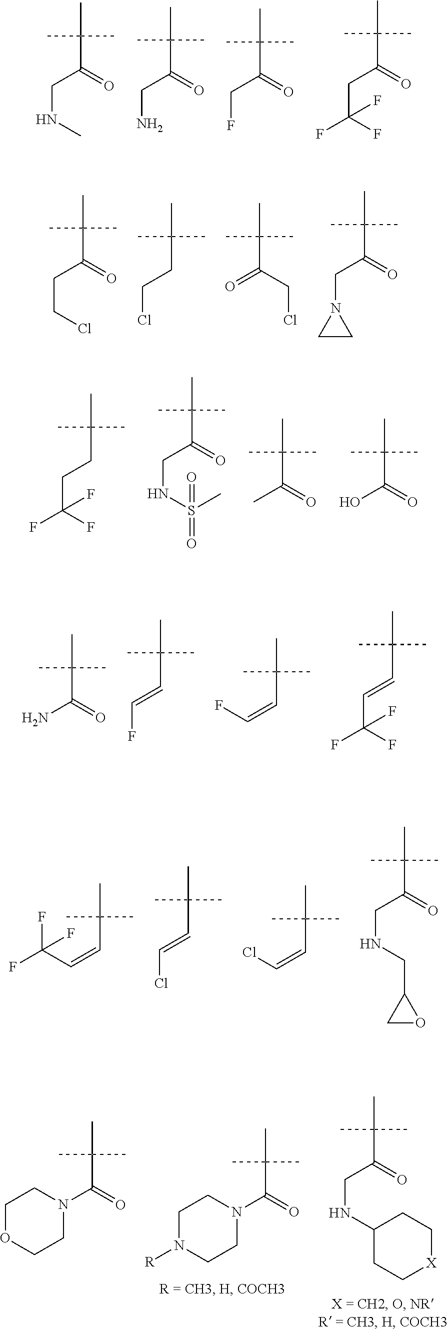 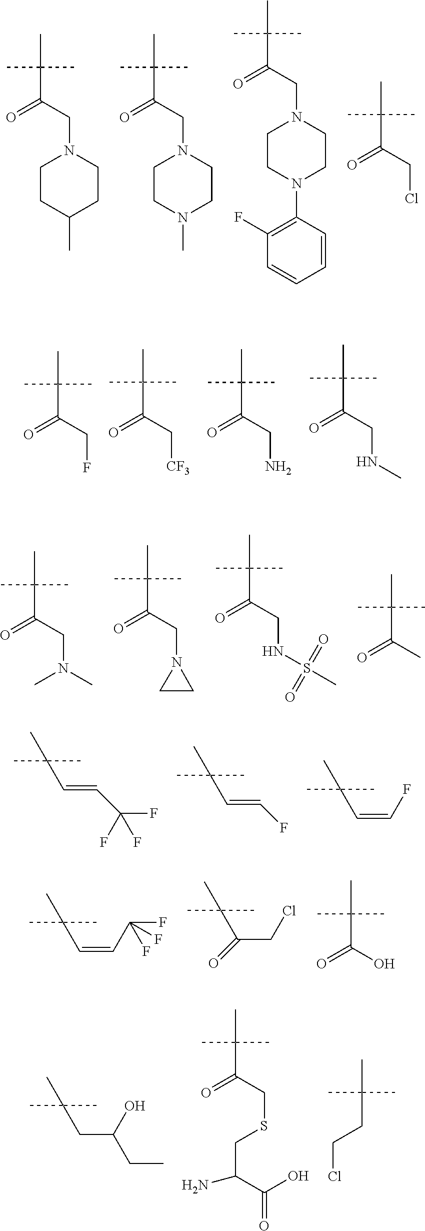 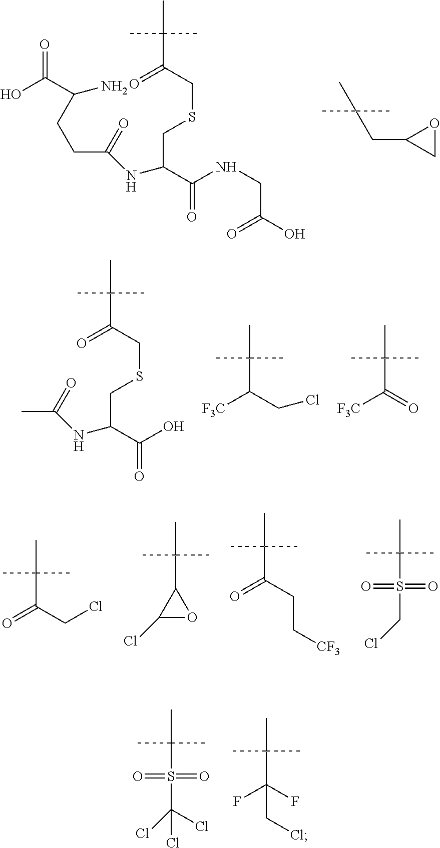
wherein Y is each independently selected from the group consisting of hydrogen, C1-C6 halo alkyl, C1-C6 halo alkoxy, C1-C6 amino alkyl, C1-C6 amino alkoxy, C3-C8 cyclo-(halo)-alkyl and wherein the alkyl or cycloalkyl group is optionally substituted with a five- or six-membered ring optionally containing at least one heteroatom selected from N, S and O, and wherein the five- or six-membered ring is optionally mono- or poly-substituted with C1-C6 alkyl, halo, C1-C6 halo alkyl, C1-C6 halo alkoxy, C1-C6 amino alkyl, C1-C6 amino alkoxy, C3-C8 cycloalkyl, C3-C8 cycloalkyl substituted with halo, amino, carboxyl or alkoxy group.

2. The compound of claim 1, wherein R4 is not hydrogen, m is zero and n is 1.

3. The compound of claim 2, wherein R4 is COY and Y is a substituted piperazine.

4. The compound of claim 2, wherein R4 is COY and Y is a halo alkyl.

5. The compound of claim 1, wherein R4 is hydrogen, m is zero, n is one and R2 is halo.

6. The compound of claim 2, wherein R4 is COY and Y is a substituted piperidine.

7. A compound selected from the group consisting of:

| Name | Compound Structure |
|---|---|
| Com- pound 1 | 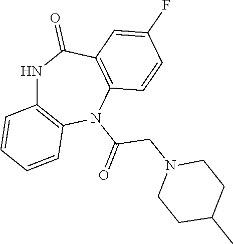 [SMILES: CC1CCN(CC(=O)N2c3ccccc3NC(=O)c3cc(F)ccc32)CC1] |
| Com- pound 2 | 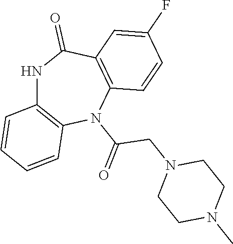 [SMILES: CN1CCN(CC(=O)N2c3ccccc3NC(=O)c3cc(F)ccc32)CC1] |
| Com- pound 3 | 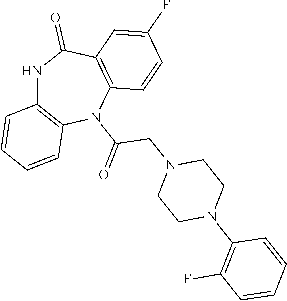 [SMILES: O=C1Nc2ccccc2N(C(=O)CN2CCN(c3ccccc3F)CC2)c2ccc(F)cc21] |
| Com- pound 4 | 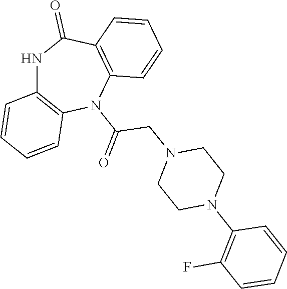 [SMILES: O=C1Nc2ccccc2N(C(=O)CN2CCN(c3ccccc3F)CC2)c2ccccc21] |
| Com- pound 5 | 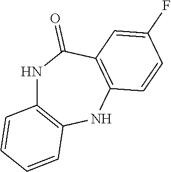 [SMILES: O=C1Nc2ccccc2Nc2ccc(F)cc21] |
| Com- pound 6 | 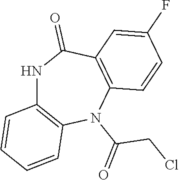 [SMILES: O=C1Nc2ccccc2N(C(=O)CCl)c2ccc(F)cc21] |
| Com- pound 7 | 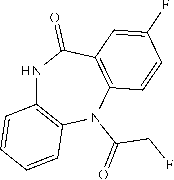 [SMILES: O=C1Nc2ccccc2N(C(=O)CF)c2ccc(F)cc21] |
| Com- pound 8 | 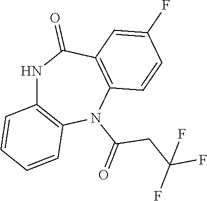 [SMILES: O=C1Nc2ccccc2N(C(=O)CC(F)(F)F)c2ccc(F)cc21] |
| Com- pound 9 | 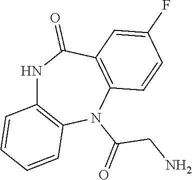 [SMILES: NCC(=O)N1c2ccccc2NC(=O)c2cc(F)ccc21] |
| Com- pound 10 | 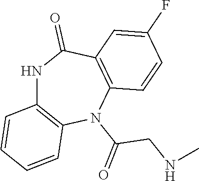 [SMILES: CNCC(=O)N1c2ccccc2NC(=O)c2cc(F)ccc21] |
| Com- pound 11 | 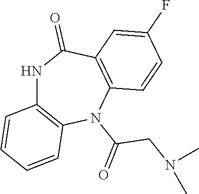 [SMILES: CN(C)CC(=O)N1c2ccccc2NC(=O)c2cc(F)ccc21] |
| Com- pound 12 | 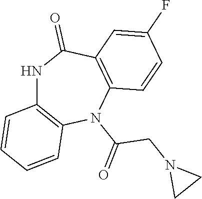 [SMILES: O=C1Nc2ccccc2N(C(=O)CN2CC2)c2ccc(F)cc21] |
| Com- pound 13 | 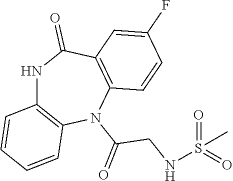 [SMILES: CS(=O)(=O)NCC(=O)N1c2ccccc2NC(=O)c2cc(F)ccc21] |
| Com- pound 14 | 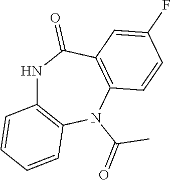 [SMILES: CC(=O)N1c2ccccc2NC(=O)c2cc(F)ccc21] |
| Com- pound 15 | 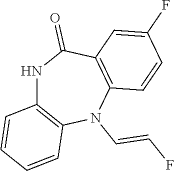 [SMILES: O=C1Nc2ccccc2N(/C=C/F)c2ccc(F)cc21] |
| Com- pound 16 | 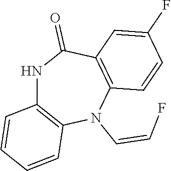 [SMILES: O=C1Nc2ccccc2N(/C=C\F)c2ccc(F)cc21] |
| Com- pound 17 | 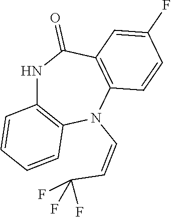 [SMILES: O=C1Nc2ccccc2N(/C=C\C(F)(F)F)c2ccc(F)cc21] |
| Com- pound 18 | 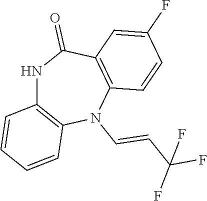 [SMILES: O=C1Nc2ccccc2N(/C=C/C(F)(F)F)c2ccc(F)cc21] |
| Com- pound 19 | 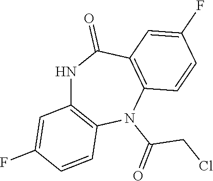 [SMILES: O=C1Nc2cc(F)ccc2N(C(=O)CCl)c2ccc(F)cc21] |
| Com- pound 20 | 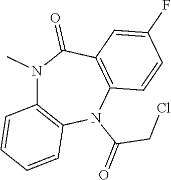 [SMILES: CN1C(=O)c2cc(F)ccc2N(C(=O)CCl)c2ccccc21] |
| Com- pound 21 | 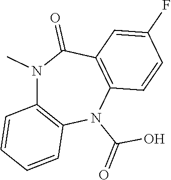 [SMILES: CN1C(=O)c2cc(F)ccc2N(C(=O)O)c2ccccc21] |
| Com- pound 22 | 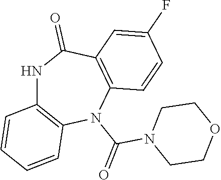 [SMILES: O=C1Nc2ccccc2N(C(=O)N2CCOCC2)c2ccc(F)cc21] |
| Com- pound 23 | 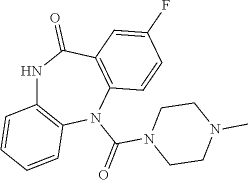 [SMILES: CN1CCN(C(=O)N2c3ccccc3NC(=O)c3cc(F)ccc32)CC1] |
| Com- pound 24 | 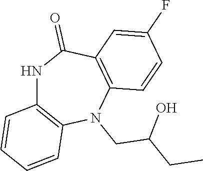 [SMILES: CCC(O)CN1c2ccccc2NC(=O)c2cc(F)ccc21] |
| Com- pound 25 | 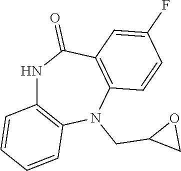 [SMILES: O=C1Nc2ccccc2N(CC2CO2)c2ccc(F)cc21] |
| Com- pound 26 | 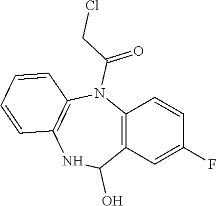 [SMILES: O=C(CCl)N1c2ccccc2NC(O)c2cc(F)ccc21] |
| Com- pound 27 | 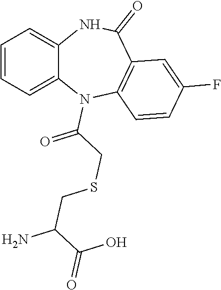 [SMILES: NC(CSCC(=O)N1c2ccccc2NC(=O)c2cc(F)ccc21)C(=O)O] |
| Com- pound 28 | 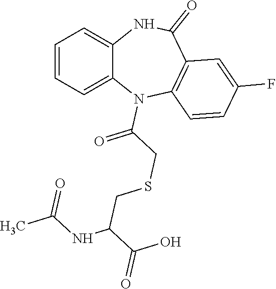 [SMILES: CC(=O)NC(CSCC(=O)N1c2ccccc2NC(=O)c2cc(F)ccc21)C(=O)O] |
| Com- pound 29 | 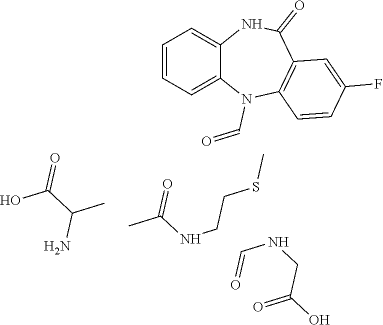 |
| Com- pound 30 | 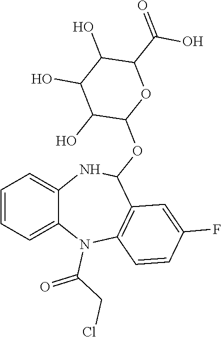 [SMILES: O=C(O)C1OC(OC2Nc3ccccc3N(C(=O)CCl)c3ccc(F)cc32)C(O)C(O)C1O] |
| Com- pound 31 | 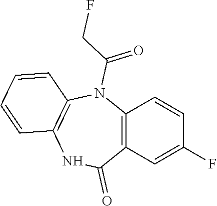 [SMILES: O=C1Nc2ccccc2N(C(=O)CF)c2ccc(F)cc21] |
| Com- pound 32 | 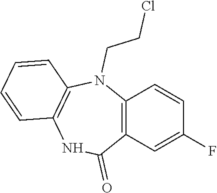 [SMILES: O=C1Nc2ccccc2N(CCCl)c2ccc(F)cc21] |
| Com- pound 33 | 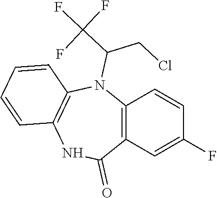 [SMILES: O=C1Nc2ccccc2N(C(CCl)C(F)(F)F)c2ccc(F)cc21] |
| Com- pound 34 | 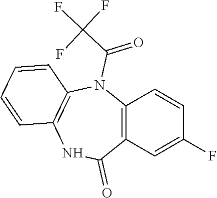 [SMILES: O=C1Nc2ccccc2N(C(=O)C(F)(F)F)c2ccc(F)cc21] |
| Com- pound 35 | 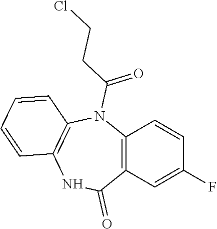 [SMILES: O=C1Nc2ccccc2N(C(=O)CCCl)c2ccc(F)cc21] |
| Com- pound 36 | 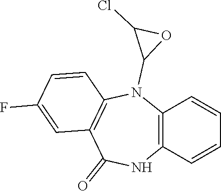 [SMILES: O=C1Nc2ccccc2N(C2OC2Cl)c2ccc(F)cc21] |
| Com- pound 37 | 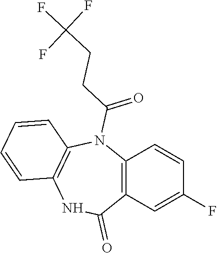 [SMILES: O=C1Nc2ccccc2N(C(=O)CCC(F)(F)F)c2ccc(F)cc21] |
| Com- pound 38 | 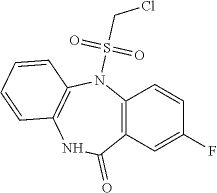 [SMILES: O=C1Nc2ccccc2N(S(=O)(=O)CCl)c2ccc(F)cc21] |
| Com- pound 39 | 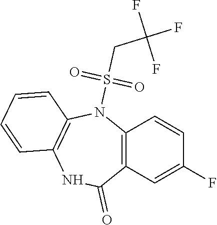 [SMILES: O=C1Nc2ccccc2N(S(=O)(=O)CC(F)(F)F)c2ccc(F)cc21] |
| Com- pound 40 | 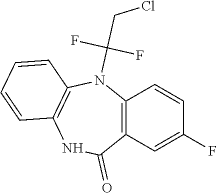 [SMILES: O=C1Nc2ccccc2N(C(F)(F)CCl)c2ccc(F)cc21] |
| Com- pound 41 | 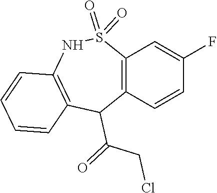 [SMILES: O=C(CCl)C1c2ccccc2NS(=O)(=O)c2cc(F)ccc21] |
| Com- pound 42 | 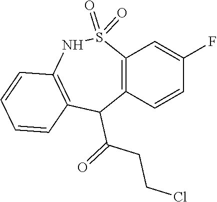 [SMILES: O=C(CCCl)C1c2ccccc2NS(=O)(=O)c2cc(F)ccc21] |
| Com- pound 43 | 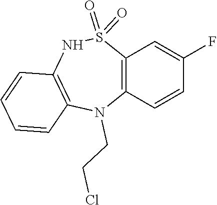 [SMILES: O=S1(=O)Nc2ccccc2N(CCCl)c2ccc(F)cc21] |
| Com- pound 44 | 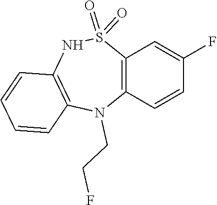 [SMILES: O=S1(=O)Nc2ccccc2N(CCF)c2ccc(F)cc21] |
| Com- pound 45 | 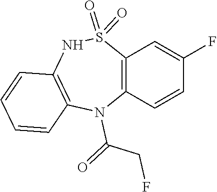 [SMILES: O=C(CF)N1c2ccccc2NS(=O)(=O)c2cc(F)ccc21] |
| Com- pound 46 | 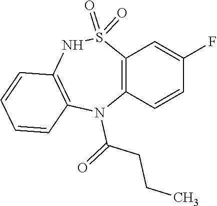 [SMILES: CCCC(=O)N1c2ccccc2NS(=O)(=O)c2cc(F)ccc21] |
| Com- pound 47 | 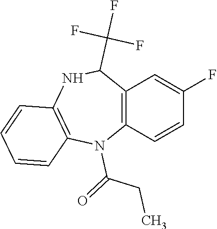 [SMILES: CCC(=O)N1c2ccccc2NC(C(F)(F)F)c2cc(F)ccc21] |
| Com- pound 48 | 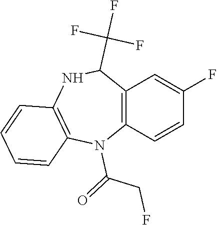 [SMILES: O=C(CF)N1c2ccccc2NC(C(F)(F)F)c2cc(F)ccc21] |
| Com- pound 49 | 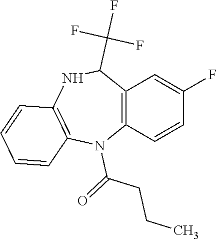 [SMILES: CCCC(=O)N1c2ccccc2NC(C(F)(F)F)c2cc(F)ccc21] |
| Com- pound 50 | 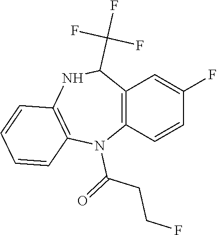 [SMILES: O=C(CCF)N1c2ccccc2NC(C(F)(F)F)c2cc(F)ccc21] |
| Com- pound 51 | 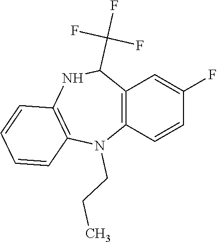 [SMILES: CCCN1c2ccccc2NC(C(F)(F)F)c2cc(F)ccc21] |
| Com- pound 52 | 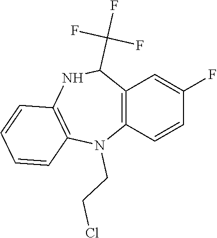 [SMILES: Fc1ccc2c(c1)C(C(F)(F)F)Nc1ccccc1N2CCCl] |
| Com- pound 53 | 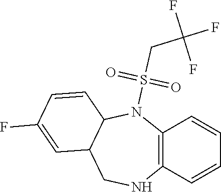 [SMILES: O=S(=O)(CC(F)(F)F)N1c2ccccc2NCC2C=C(F)C=CC21] |
| Com- pound 54 | 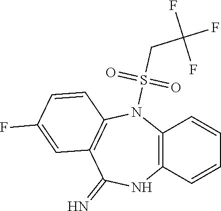 [SMILES: N=C1Nc2ccccc2N(S(=O)(=O)CC(F)(F)F)c2ccc(F)cc21] |
| Com- pound 55 | 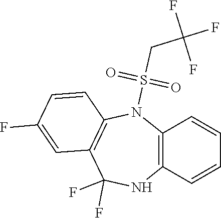 [SMILES: O=S(=O)(CC(F)(F)F)N1c2ccccc2NC(F)(F)c2cc(F)ccc21] |
| Com- pound 56 | 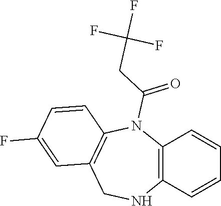 [SMILES: O=C(CC(F)(F)F)N1c2ccc(F)cc2CNc2ccccc21] |
| Com- pound 57 | 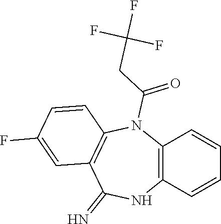 [SMILES: N=C1Nc2ccccc2N(C(=O)CC(F)(F)F)c2ccc(F)cc21] |
| Com- pound 58 | 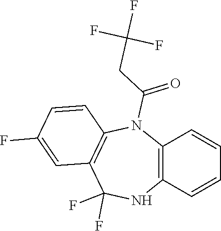 [SMILES: O=C(CC(F)(F)F)N1c2ccccc2NC(F)(F)c2cc(F)ccc21] |
| Com- pound 59 | 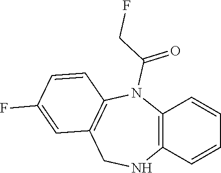 [SMILES: O=C(CF)N1c2ccc(F)cc2CNc2ccccc21] |
| Com- pound 60 | 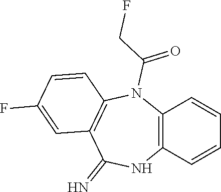 [SMILES: N=C1Nc2ccccc2N(C(=O)CF)c2ccc(F)cc21] |
| Com- pound 61 | 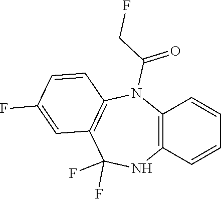 [SMILES: O=C(CF)N1c2ccccc2NC(F)(F)c2cc(F)ccc21] |
| Com- pound 62 | 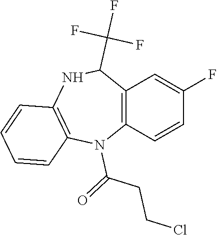 [SMILES: O=C(CCCl)N1c2ccccc2NC(C(F)(F)F)c2cc(F)ccc21] |

8. The compound of claim 1, wherein said compound has an IC50 value of about 2 μm.

9. The compound of claim 1, wherein said compound is capable of reducing the expression of IL-1β by at least 50%.

10. The compound of claim 1, wherein said compound is 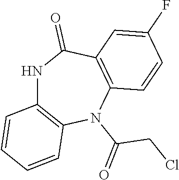 [SMILES: O=C1Nc2ccccc2N(C(=O)CCl)c2ccc(F)cc21]
or a pharmaceutically acceptable salt thereof.

11. A compound of formula I(a) 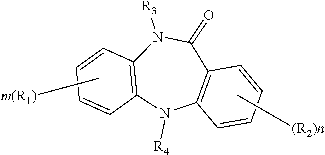
wherein R1 and R2 are each independently selected from the group consisting of hydrogen, —CO-alkyl, hydroxyl, halo, halo alkyl (C1-C6), trihalo alkyl (C1-C6), halo alkoxy, amino, C1-C6-alkyl-amino; wherein m and n are integers having each independently a value of 0, 1, 2, 3 or 4;
wherein R3 is each independently selected from the group consisting of hydrogen, C1-C6 alkyl, trilhalo alkyl (C1-C6), and —CO-alkyl;
wherein R4 is each independently one of hydrogen or COY with the proviso that R4 is not hydrogen when R3 is hydrogen, except that R4 and R3 may both be hydrogen when either R1 or R2 is halo or 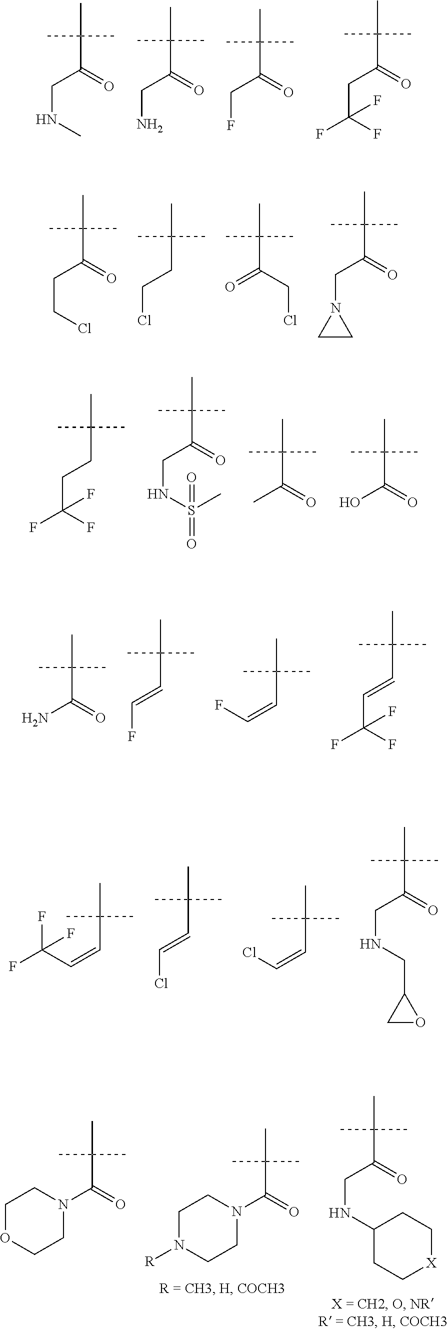 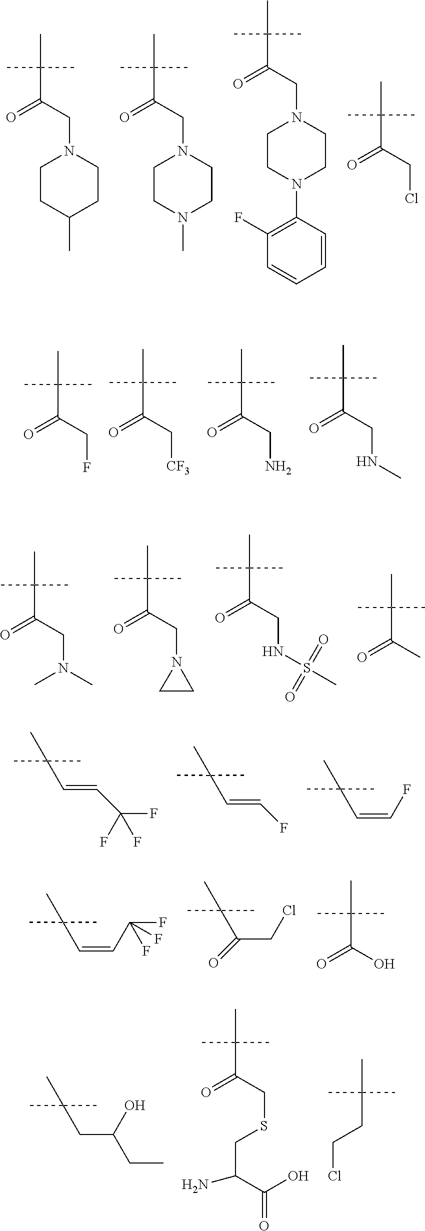 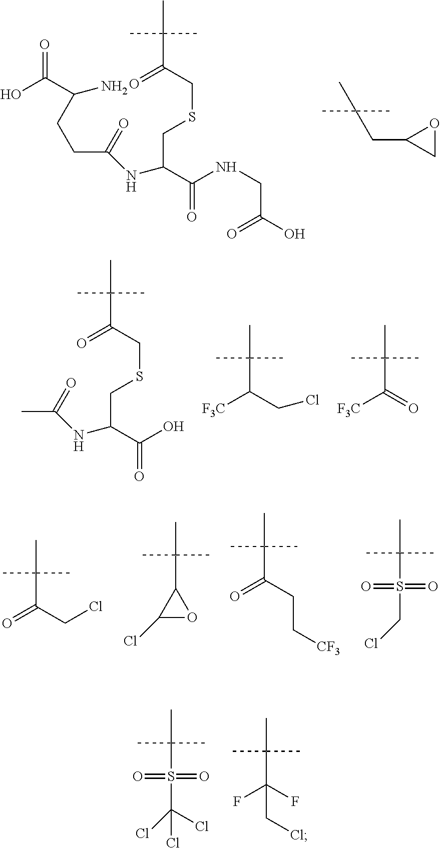
wherein Y is each independently selected from the group consisting of hydrogen, C1-C6 halo alkyl, C1-C6 halo alkoxy, C1-C6 amino alkyl, C1-C6 amino alkoxy, C3-C8 cyclo-(halo)-alkyl and wherein the alkyl or cycloalkyl group is optionally substituted with a five- or six-membered ring optionally containing at least one heteroatom selected from N, S and O, and wherein the five- or six-membered ring is optionally mono- or poly-substituted with C1-C6 alkyl, halo, C1-C6 halo alkyl, C1-C6 halo alkoxy, C1-C6 amino alkyl, C1-C6 amino alkoxy, C3-C8 cycloalkyl, C3-C8 cycloalkyl substituted with halo, amino, carboxyl or alkoxy group.

12. A compound of formula I(b) 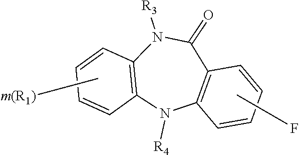
wherein R1 is each independently selected from the group consisting of hydrogen, —CO— alkyl, hydroxyl, halo, halo alkyl (C1-C6), trihalo alkyl (C1-C6), halo alkoxy, amino, C1-C6-alkyl-amino; wherein m is an integer having each independently a value of 0, 1, 2, 3 or 4;
wherein R3 is each independently selected from the group consisting of hydrogen, C1-C6 alkyl, trilhalo alkyl (C1-C6), and —CO-alkyl;
wherein R4 is each independently one of hydrogen or COY or 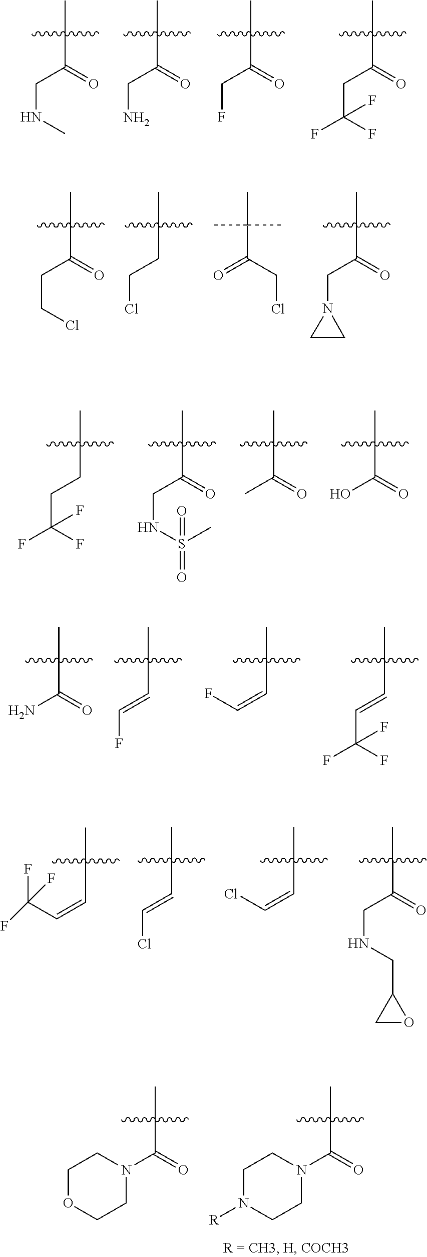 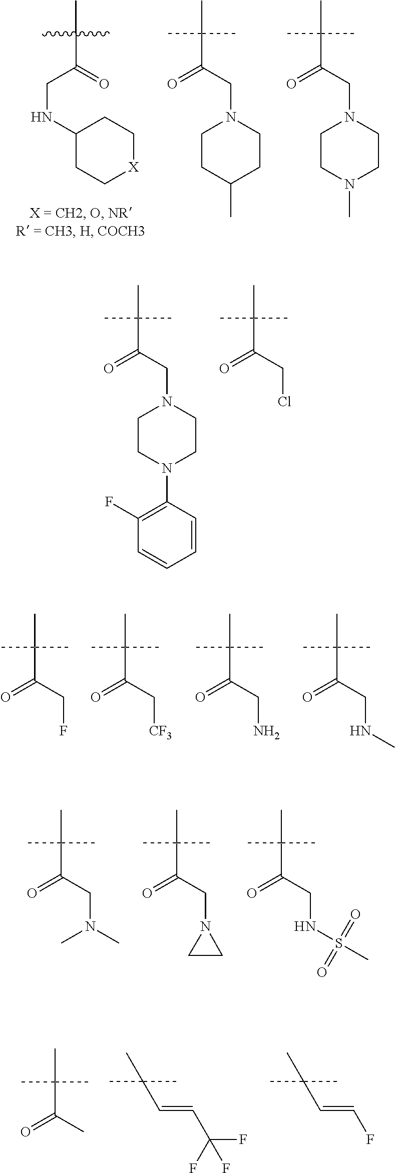 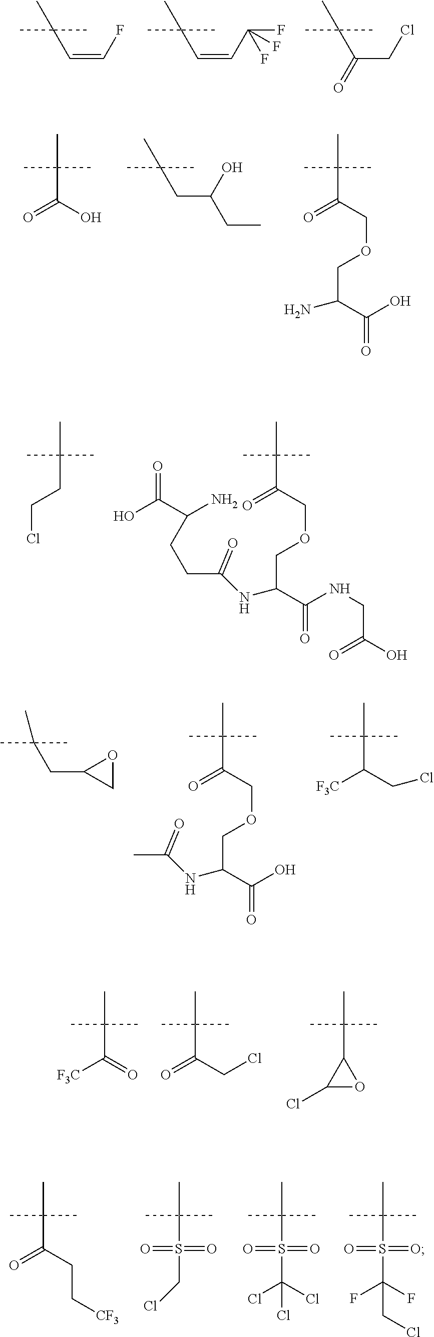
wherein Y is each independently selected from the group consisting of hydrogen, C1-C6 halo alkyl, C1-C6 halo alkoxy, C1-C6 amino alkyl, C1-C6 amino alkoxy, C3-C8 cyclo-(halo)-alkyl and wherein the alkyl or cycloalkyl group is optionally substituted with a five- or six-membered ring optionally containing at least one heteroatom selected from N, S and O, and wherein the five- or six-membered ring is optionally mono- or poly-substituted with C1-C6 alkyl, halo, C1-C6 halo alkyl, C1-C6 halo alkoxy, C1-C6 amino alkyl, C1-C6 amino alkoxy, C3-C8 cycloalkyl, C3-C8 cycloalkyl substituted with halo, amino, carboxyl or alkoxy group.

13. A compound of formula I(c) 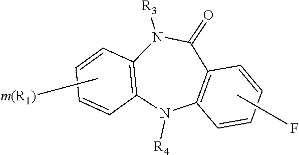
wherein R1 is hydrogen and m is 1,
wherein R3 is hydrogen, and
wherein R4 is each independently one of hydrogen or COY or 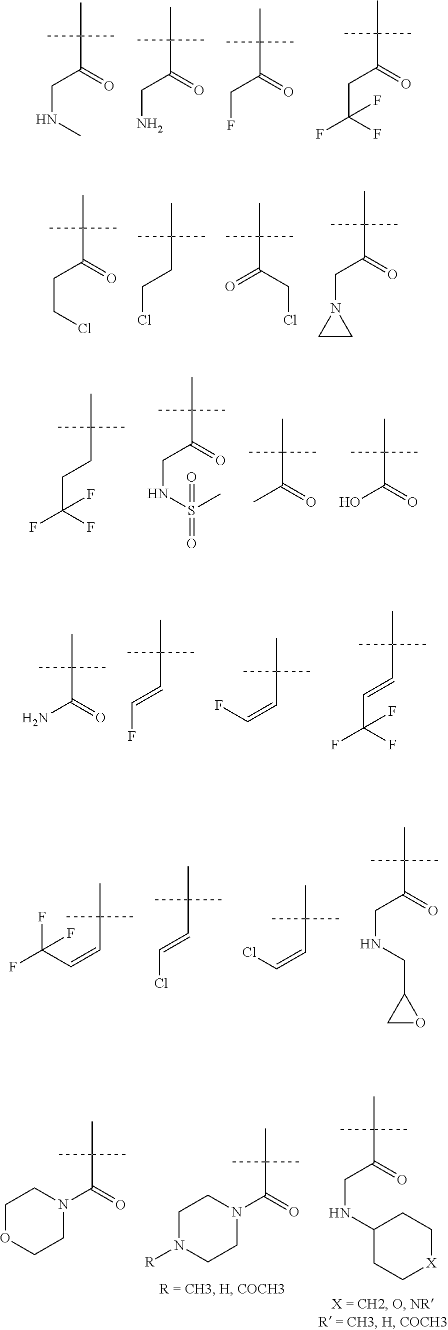 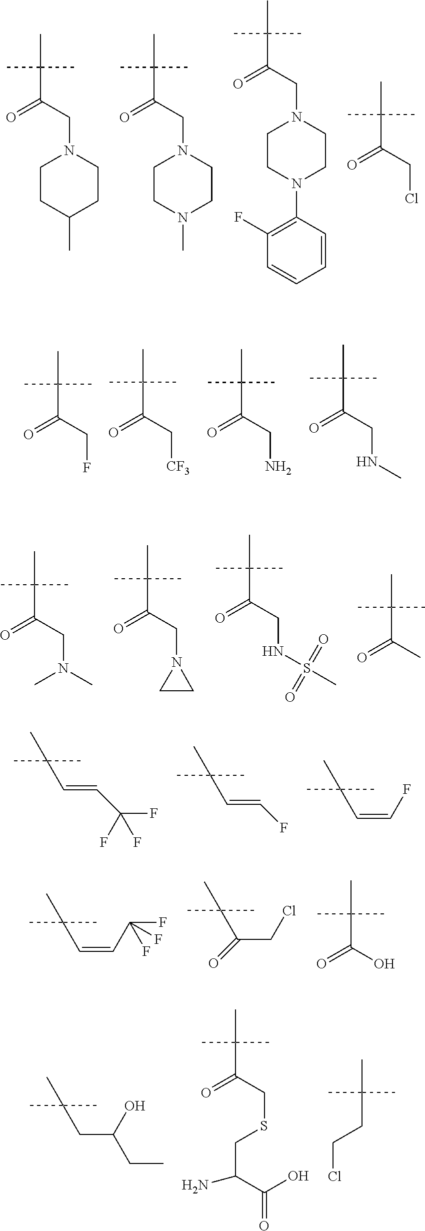 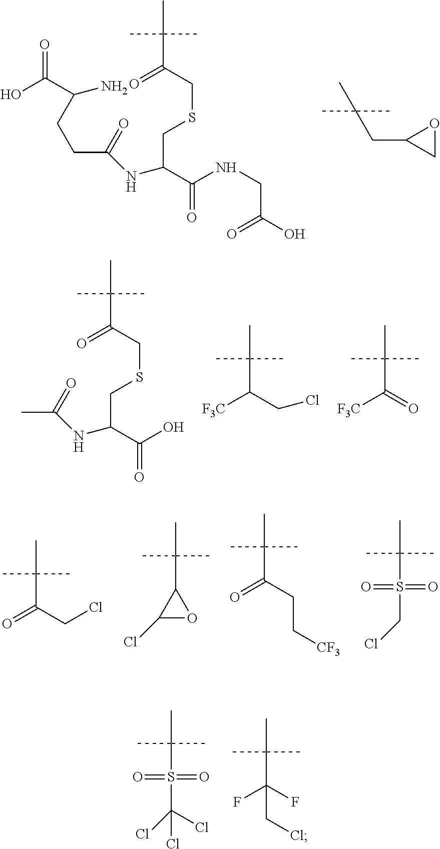
wherein Y is each independently selected from the group consisting of hydrogen, C1-C6 halo alkyl, C1-C6 halo alkoxy, C1-C6 amino alkyl, C1-C6 amino alkoxy, C3-C8 cyclo-(halo)-alkyl and wherein the alkyl or cycloalkyl group is optionally substituted with a five- or six-membered ring optionally containing at least one heteroatom selected from N, S and O, and wherein the five- or six-membered ring is optionally mono- or poly-substituted with C1-C6 alkyl, halo, C1-C6 halo alkyl, C1-C6 halo alkoxy, C1-C6 amino alkyl, C1-C6 amino alkoxy, C3-C8 cycloalkyl, C3-C8 cycloalkyl substituted with halo, amino, carboxyl or alkoxy group.

14. A compound selected from a group consisting of:
2-fluoro-5-(2-(4-methylpiperidin-1-yl)acetyl)-5,10-dihydro-11H-dibenzo[b,e][1,4]diazepin-11-one, 2-fluoro-5-(2-(4-methylpiperidin-1-yl)acetyl)-5,10-dihydro-11H-dibenzo[b,e][1,4]diazepin-11-one, 2-fluoro-5-(2-(4-(2-fluorophenyl)piperazin-1-yl)acetyl)-5,10-dihydro-11H-dibenzo[b,e][1,4]diazepin-11-one, 5-(2-chloroacetyl)-5,10-dihydro-11H-dibenzo[b,e][1,4]diazepin-11-one, 2-fluoro-5,10-dihydro-11H-dibenzo[b,e][1,4]diazepin-11-one, 5-(2-chloroacetyl)-2-fluoro-5,10-dihydro-11H-dibenzo[b,e][1,4]diazepin-11-one, 2-fluoro-5-(fluoroacetyl)-5,10-dihydro-11H-dibenzo[b,e][1,4]diazepin-11-one, 2-fluoro-5-(3,3,3-trifluoropropanoyl)-5,10-dihydro-11H-dibenzo[b,e][1,4]diazepin-11-one, 5-(aminoacetyl)-2-fluoro-5,10-dihydro-11H-dibenzo[b,e][1,4]diazepin-11-one, 2-fluoro-5-[(methylamino)acetyl]-5,10-dihydro-11H-dibenzo[b,e][1,4]diazepin-11-one, 5-[(dimethylamino)acetyl]-2-fluoro-5,10-dihydro-11H-dibenzo[b,e][1,4]diazepin-11-one, 5-[(aziridin-1-yl)acetyl]-2-fluoro-5,10-dihydro-11H-dibenzo[b,e][1,4]diazepin-11-one, N-[2-(2-fluoro-11-oxo-10,11-dihydro-5H-dibenzo[b,e][1,4]diazepin-5-yl)-2-oxoethyl]methanesulfonamide, 5-acetyl-2-fluoro-5,10-dihydro-11H-dibenzo[b,e][1,4]diazepin-11-one, 2-fluoro-5-[(E)-2-fluoroethenyl]-5,10-dihydro-11H-dibenzo[b,e][1,4]diazepin-11-one, 2-fluoro-5-[(Z)-2-fluoroethenyl]-5,10-dihydro-11H-dibenzo[b,e][1,4]diazepin-11-one, 2-fluoro-5-[(1Z)-3,3,3-trifluoroprop-1-en-1-yl]-5,10-dihydro-11H-dibenzo[b,e][1,4]diazepin-11-one, 2-fluoro-5-[(1E)-3,3,3-trifluoroprop-1-en-1-yl]-5,10-dihydro-11H-dibenzo[b,e][1,4]diazepin-11-one, 5-(chloroacetyl)-2-fluoro-10-methyl-5,10-dihydro-11H-dibenzo[b,e][1,4]diazepin-11-one, 5-(chloroacetyl)-2-fluoro-10-methyl-5,10-dihydro-11H-dibenzo[b,e][1,4]diazepin-11-one, 2-fluoro-10-methyl-11-oxo-10,11-dihydro-5H-dibenzo[b,e][1,4]diazepine-5-carboxylic acid, 2-fluoro-5-(morpholine-4-carbonyl)-5,10-dihydro-11H-dibenzo[b,e][1,4]diazepin-11-one, 2-fluoro-5-(4-methylpiperazine-1-carbonyl)-5,10-dihydro-11H-dibenzo[b,e][1,4]diazepin-11-one, 2-fluoro-5-(2-hydroxybutyl)-5,10-dihydro-11H-dibenzo[b,e][1,4]diazepin-11-one, 2-fluoro-5-[(oxiran-2-yl)methyl]-5,10-dihydro-11H-dibenzo[b,e][1,4]diazepin-11-one, 2-chloro-1-(2-fluoro-11-hydroxy-10,11-dihydro-5H-dibenzo[b,e][1,4]diazepin-5-yl)ethan-1-one, 2-amino-3-{[2-(2-fluoro-11-oxo-10,11-dihydro-5H-dibenzo[b,e][1,4]diazepin-5-yl)-2-oxoethyl]sulfanyl}propanoic acid, 2-acetamido-3-{[2-(2-fluoro-11-oxo-10,11-dihydro-5H-dibenzo[b,e][1,4]diazepin-5-yl)-2-oxoethyl]sulfanyl}propanoic acid, 2-amino-5-((1-((carboxymethyl)amino)-3-((2-(2-fluoro-11-oxo-10,11-dihydro-5H-dibenzo[b,e][1,4]diazepin-5-yl)-2-oxoethyl)thio)-1-oxopropan-2-yl)amino)-5-oxopentanoic acid, 6-((5-(2-chloroacetyl)-2-fluoro-10,11-dihydro-5H-dibenzo[b,e][1,4]diazepin-11-yl)oxy)-3,4,5-trihydroxytetrahydro-2H-pyran-2-carboxylic acid, 2-fluoro-5-(fluoroacetyl)-5,10-dihydro-11H-dibenzo[b,e][1,4]diazepin-11-one, 5-(2-chloroethyl)-2-fluoro-5,10-dihydro-11H-dibenzo[b,e][1,4]diazepin-11-one, 5-(3-chloro-1,1,1-trifluoropropan-2-yl)-2-fluoro-5,10-dihydro-11H-dibenzo[b,e][1,4]diazepin-11-one, 2-fluoro-5-(trifluoroacetyl)-5,10-dihydro-11H-dibenzo[b,e][1,4]diazepin-11-one, 5-(3-chloropropanoyl)-2-fluoro-5,10-dihydro-11H-dibenzo[b,e][1,4]diazepin-11-one, 5-(3-chlorooxiran-2-yl)-2-fluoro-5,10-dihydro-11H-dibenzo[b,e][1,4]diazepin-11-one, 2-fluoro-5-(4,4,4-trifluorobutanoyl)-5,10-dihydro-11H-dibenzo[b,e][1,4]diazepin-11-one, 5-(chloromethanesulfonyl)-2-fluoro-5,10-dihydro-11H-dibenzo[b,e][1,4]diazepin-11-one, 2-fluoro-5-(2,2,2-trifluoroethanesulfonyl)-5,10-dihydro-11H-dibenzo[b,e][1,4]diazepin-11-one, 5-(2-chloro-1,1-difluoroethyl)-2-fluoro-5,10-dihydro-11H-dibenzo[b,e][1,4]diazepin-11-one, 11-(chloroacetyl)-3-fluoro-6,11-dihydro-5H-5λ6-dibenzo[c,f][1,2]thiazepine-5,5-dione, 11-(3-chloropropanoyl)-3-fluoro-6,11-dihydro-5H-5λ6-dibenzo[c,f][1,2,5]thiadiazepine-5,5-dione, 11-(2-chloroethyl)-3-fluoro-6,11-dihydro-5H-5λ6-dibenzo[c,f][1,2,5]thiadiazepine-5,5-dione, 3-fluoro-11-(2-fluoroethyl)-6,11-dihydro-5H-5λ6-dibenzo[c,f][1,2,5]thiadiazepine-5,5-dione, 3-fluoro-11-(fluoroacetyl)-6,11-dihydro-5H-5λ6-dibenzo[c,f][1,2,5]thiadiazepine-5,5-dione, 11-butanoyl-3-fluoro-6,11-dihydro-5H-5λ6-dibenzo[c,f][1,2,5]thiadiazepine-5,5-dione, 1-[2-fluoro-11-(trifluoromethyl)-10,11-dihydro-5H-dibenzo[b,e][1,4]diazepin-5-yl]propan-1-one, 2-fluoro-1-[2-fluoro-11-(trifluoromethyl)-10,11-dihydro-5H-dibenzo[b,e][1,4]diazepin-5-yl]ethan-1-one, 1-[2-fluoro-11-(trifluoromethyl)-10,11-dihydro-5H-dibenzo[b,e][1,4]diazepin-5-yl]butan-1-one, 3-fluoro-1-[2-fluoro-11-(trifluoromethyl)-10,11-dihydro-5H-dibenzo[b,e][1,4]diazepin-5-yl]propan-1-one, 2-fluoro-5-propyl-11-(trifluoromethyl)-10,11-dihydro-5H-dibenzo[b,e][1,4]diazepine, 5-(2-chloroethyl)-2-fluoro-11-(trifluoromethyl)-10,11-dihydro-5H-dibenzo[b,e][1,4]diazepine, 2-fluoro-5-(2,2,2-trifluoroethanesulfonyl)-5,10,11,11a-tetrahydro-4aH-dibenzo[b,e][1,4]diazepine, 2-fluoro-5-(2,2,2-trifluoroethanesulfonyl)-5,10-dihydro-11H-dibenzo[b,e][1,4]diazepin-11-imine, 2,11,11-trifluoro-5-(2,2,2-trifluoroethanesulfonyl)-10,11-dihydro-5H-dibenzo[b,e][1,4]diazepine, 3,3,3-trifluoro-1-(2-fluoro-10,11-dihydro-5H-dibenzo[b,e][1,4]diazepin-5-yl)propan-1-one, 3,3,3-trifluoro-1-(2-fluoro-11-imino-10,11-dihydro-5H-dibenzo[b,e][1,4]diazepin-5-yl)propan-1-one, 3,3,3-trifluoro-1-(2,11,11-trifluoro-10,11-dihydro-5H-dibenzo[b,e][1,4]diazepin-5-yl)propan-1-one, 2-fluoro-1-(2-fluoro-10,11-dihydro-5H-dibenzo[b,e][1,4]diazepin-5-yl)ethan-1-one, 2-fluoro-1-(2-fluoro-11-imino-10,11-dihydro-5H-dibenzo[b,e][1,4]diazepin-5-yl)ethan-1-one, 2-fluoro-1-(2,11,11-trifluoro-10,11-dihydro-5H-dibenzo[b,e][1,4]diazepin-5-yl)ethan-1-one, and 3-chloro-1-[2-fluoro-11-(trifluoromethyl)-10,11-dihydro-5H-dibenzo[b,e][1,4]diazepin-5-yl]propan-1-one.
